(19) United States
(12) Patent Application Publication
(10) Pub. No.: US 2024/0093152 A1
(43) Pub. Date: Mar. 21, 2024

(54) ECTOPIC CELLULAR GROWTH FACTOR EXPRESSION FOR LOW-COST PRODUCTION OF CELL-CULTURED FOODS

(71) Applicant: Trustees of Tufts College, Medford, MA (US)

(72) Inventors: David L. Kaplan, Concord, MA (US); Andrew Stout, Cambridge, MA (US); John Yuen, JR., Somerville, MA (US); Natalie Rubio, Medford, MA (US)

(21) Appl. No.: 18/252,714

(22) PCT Filed: Nov. 12, 2021
(86) PCT No.: PCT/US21/72383

§ 371 Date: May 11, 2023

Related U.S. Application Data
(60) Provisional application No. 63/112,682, filed on Nov. 12, 2020.

(51) Int. Cl. C12N 5/077; A23L 13/00; C12N 9/22; C12N 15/63

(52) U.S. Cl. CPC C12N 5/0652; A23L 13/00; C12N 9/22; C12N 15/63; C12N 2310/20; C12N 2513/00

(57) ABSTRACT

The present disclosure relates to cultured tissue, methods for production of self-sufficient modified cells for producing cultured tissue. Further, the disclosure provides methods of generating cultured meat products without exogenous addition of growth hormones.

### CROSS-REFERENCE TO RELATED APPLICATIONS

This application is related to, claims priority to, and incorporates herein by reference for all purposes U.S. Provisional Patent Application 63/112,682, filed Nov. 12, 2020.

### SEQUENCE LISTING

A Sequence Listing accompanies this application and is submitted as an ASCII text file of the sequence listing named “166118_01116_ST25.txt” which is 406,692 bytes in size and was created on Nov. 12, 2021. The sequence listing is electronically submitted via EFS-Web with the application and is incorporated herein by reference in its entirety.

### BACKGROUND

Cultured meat, or meat produced through cell culture and tissue engineering, offers the potential to drastically alter our world's meat production system by addressing the environmental, ethical, and health concerns associated with modern animal agriculture. The high costs of current cell culture media are prohibitive to this effort as it requires many exogenous recombinant proteins and growth factors for propagation and expansion.

Therefore, bringing down the costs of this media is a key hurdle facing cultured meat's development and reaching price parity with conventional meats. The proposed approach offers a promising option, as it completely eliminates the need for the most expensive components of cell culture media, thereby drastically lowering costs.

### SUMMARY

The present invention provides modified cells that can grow in minimal media for use in cultured meat, methods of making and uses thereof. Further, the invention provides a cultured meat product comprising in vitro grown cells in minimal media. Methods of culturing cells in minimal media to make a cultured meat product are also provided.

In one aspect, the disclosure provides a modified non-human cell ectopically expressing two or more growth factors or cytokines or receptors thereof that promote cell growth, wherein the two or more factors are selected from Table 1. In some aspects, the growth of these cells do not require exogenous supplementation with growth factors.

In another embodiment, a composition comprising the modified non-human cells described herein are provided.

In a further embodiment, a meat product comprising a population of the cells described herein is provided.

In a further aspect, a method of producing a meat product in in vitro culture is provided. The method comprises culturing a population of the modified non-human cells described herein in minimal culture medium for a sufficient time to increase the number of cells, whereby the method produces a non-human animal tissue suitable for human and/or animal consumption and wherein the minimal media does not contain exogenous growth factors.

In a further aspect, the disclosure provides a method of producing a population of modified cells for making a food product, the method comprising: (a) expressing two or more factors of Table 1 in cells; (b) culturing the cells of (a) in minimal medium for a sufficient time to promote growth of cells to a sufficient number to produce a food product; wherein the minimal media does not contain exogenous growth factors.

In another aspect, the disclosure provides a method of producing a meat product in in vitro culture, the method comprising: co-culturing a population of target cells and a feeder cell population comprising the modified non-human cells described herein in minimal culture medium for a sufficient time to increase the number of target cells, whereby the method produces a non-human animal tissue suitable for human and/or animal consumption and wherein the minimal media does not contain exogenous growth factors and wherein the target cell is not genetically modified. In another aspect, a food product comprising a population of the target cells produced by the method described herein is provided.

In a further aspect, compositions and food products comprising cells made by the methods described herein are provided.

### BRIEF DESCRIPTION OF THE DRAWINGS

FIG. 1. Growth factor expression cassette for the constitutive endogenous production of bovine FGF-2, bovine TGFb3, NRG1, and GFP all connected by ribosomal skipping sequences. Additionally, constitutive expression of a puromycin resistance gene enables selection of engineered cells.

FIG. 2. Brightfield and green-fluorescence images of bovine satellite cells engineered with the construct in FIG. 1 (as well as a Tet-inducible construct) two days after transfection and following ten days of selection in puromycin-containing media. Images show stable expression of GFP and growth factors in engineered cells.

FIG. 3. Efficacy of ectopic growth factor expression in serum-free and growth-factor free media. (A) composition of B8 serum free media and B5 serum free media without any FGF-2, NRG-1, or TGFb3. (B) Growth over four days of bovine satellite cells engineered with the construct from FIG. 1 with a constitutive promoter (CMV), or with an inducible promoter (Tet). Cells expressing growth factor genes (Tet+Dox & CMV) showed improved growth compared with cells not expressing growth factor genes (Tet-Dox). This is particularly true for constitutive expression of growth factor genes.

FIG. 4. Efficacy of ectopic growth factor expression in optimized serum-free media containing added recombinant albumin, but lacking growth factors. Results show that cells engineered to express growth factors (“pCMV-3GF-GP”) grow over ten days in culture without growth-factors added, and that this growth is substantially and significantly (p<0.01) more than that of cells not engineered to express growth factors (“pCMV-GP”).

FIG. 5. Expression cassette for the constitutive endogenous production of bovine FGF-2, bovine FGF receptor 1 (FGFR1) and GFP all connected by ribosomal skipping sequences. Additionally, constitutive expression of a puromycin resistance gene enables selection of engineered cells.

FIG. 6. (A) Expression cassette for the constitutive endogenous production of bovine FGF-2, bovine FGF receptor 1 (FGFR1), long-range IGF-1, bovine transferrin and GFP all connected by ribosomal skipping sequences. This construct could potentially eliminate the need for FGF, Insulin, and Transferrin in B8 or Beefy-9 media, which, in concert with the potential elimination of NRG and TGF (Supplementary FIG. 1), could result in a highly simplified, highly cost-effective medium for cell expansion. (B) Bovine satellite cells engineered to express this cassette and cultured over 13 days show good gene expression, as indicated by GFP fluorescence.

FIG. 7. Growth of bovine satellite cells over 3 & 4 days in B8 media (indicated by blue arrows) or media containing higher or lower concentrations of NRG1 and TGFb3. Results suggest that it might be possible to remove these growth factors from B8 media (or potentially Beefy-9) without negatively affecting cell growth. This would further imply that ectopic expression of these growth factors may not be necessary to ameliorate growth-factor requirements in serum-free media.

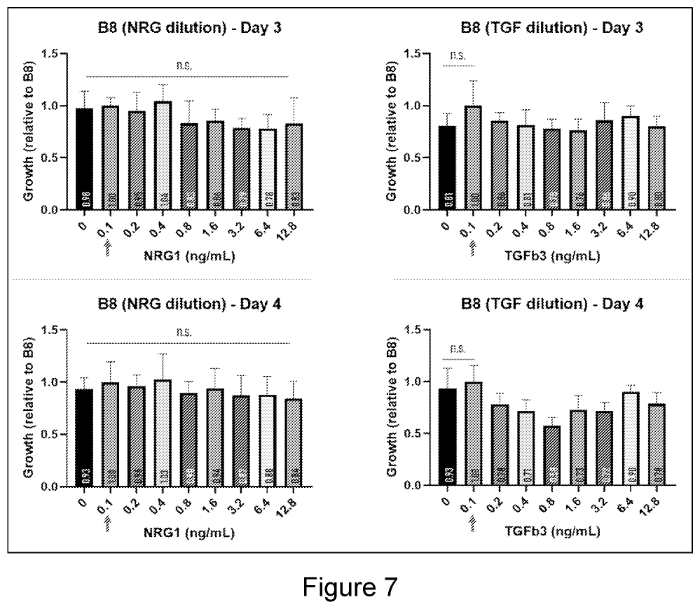

### DETAILED DESCRIPTION

Before the present invention is described in further detail, it is to be understood that the invention is not limited to the particular embodiments described. It is also understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

Cultured meat (also called in vitro, cell-based, cultivated, lab grown meat) prepared using tissue and bioengineering techniques in vitro is another alternative to traditional animal agriculture. By directly growing meat (muscle and fat tissue) in vitro, energy and nutrients may be more efficiently focused on the outcome. The time frame to generate cultured meat tissues in vitro is also thought to be faster compared to traditional animal agriculture, and may only require weeks as opposed to months or years for pork and beef, for example. Moreover, tight control over cell biology during tissue cultivation, as well as the production process, allows for the fine tuning of nutritional parameters by engineering muscle or fat cells to produce vital nutrients that would otherwise not be found (or found only at low concentrations) in conventional meat. Thus, cultured meat production systems may offer healthier, more efficient, and more environmentally friendly alternatives to animal-derived meats.

The technology disclosed herein exploits genetic strategies to generate “self-sufficient” cell lines that endogenously produce all of the requisite signaling molecules for growth in low cost, chemically defined cell culture media. The “self-sufficient” cell lines described herein are genetically modified or engineered to endogenously produce required molecules for growth in minimal tissue culture media. Specifically, ectopic expression of growth factors (e.g., fibroblast growth factor (FGF), transforming growth factor (TGFb), neuregulin (NRG), Insulin-like growth factor (IGF), etc.), growth factor receptors (FGF receptors, TGF receptors, NRG receptors, IGF receptors, etc.) and/or signaling/nutrient transport proteins (e.g., insulin, albumin, transferrin, etc.) ameliorate the need for the exogenous inclusion of these proteins in cell culture media. As growth factors and signaling proteins contribute over 95% of the cost of standard cell culture media, this invention could drastically lower the cost of production of cultured meats producing products that could compete pricewise with animal agriculture.

In the instant disclosure, stem cell lines from food-relevant tissues (e.g., muscle, fat, liver, connective tissue) of relevant animal species (e.g., bovine, porcine, piscine, galline) are engineered (modified) to express the aforementioned genes (and potentially others) constitutively or under controllable promoter systems allowing the necessary protein growth and propagation factors to be endogenously expressed by the cell. This allows for minimal media to be used for tissue culture, preferably wherein the minimal media does not comprise exogenous growth factors. Options for genetic engineering include insertion of cassettes, i.e., polynucleotide encoding the signaling/growth molecules (e.g. through CRISPR/Cas9, transposon-mediated, or recombinase-mediated genetic insertion), or by genetic activation of the native genes in cells.

Endogenous growth factors and signaling molecules have been produced in CHO cells and 3T3 fibroblasts to abrogate the need for these components in cell culture media (Pak et al. “Super-CHO-A cell line capable of autocrine growth under fully defined protein-free conditions”. Cytotechnology 1996 January; 22(1-3):139-46, DOI: 10.1007/BF00353933; Z Pietrzkowski, Z. et al. “Constitutive expression of insulin-like growth factor 1 and insulin-like growth factor 1 receptor abrogates all requirements for exogenous growth factors”. Cell Growth Differ. 1992 April; 3(4):199-205. PMID 1325181; U.S. Pat. No. 6,797,515B2, each of which are incorporated herein by reference). Specifically, these cells were engineered to express insulin or IGF, IGFR, and transferrin. While this has been demonstrated for these proteins and in CHO and 3T3 cells for pharmaceutical applications, the use in food production is novel.

### Methods of Generating “Self-Sufficient” Cells for Low-Cost Production of Cell-Cultured Foods

In one aspect of the current disclosure, modified non-human cells ectopically expressing two or more growth factors or cytokines or receptors thereof that promote cell growth are provided. In some embodiments, the two or more factors are selected from the factors listed in Table 1.

As used herein, “factors” refer to the growth factors, cytokines, or other proteins used to generate self-sufficient cells. As used herein, “self-sufficient” refers to cells that require minimal exogenous supplementation with growth factor, or more preferably, no exogenous supplementation with growth factors. In some embodiments, factors refers to the proteins listed in Table 1. In certain contexts, factors refer to the nucleotide sequence encoding the proteins listed in Table 1.

As used herein, “ectopic” expression is refers to expression of mRNA or proteins under the control of a non-native heterologous promoter and/or enhancer. The term “ectopic” further encompasses exogenous polynucleotides that are introduced into the cell and capable of expressing the factors described herein. Thus, in some embodiments, ectopic expression is achieved through exogenous polynucleotide sequences integrated into the genome of the cells of the instant disclosure, e.g., through transposons, viral transduction, or recombination. In some embodiments, exogenous expression is achieved through polynucleotide sequences that are not integrated into the genome of the cells of the instant disclosure, but are expressed episomally. Episomes, in eukaryotes, are extrachromosomal, closed circular DNA molecules of a plasmid or a viral genome origin, that are replicated autonomously in the host cell and therefore, they bear significant vector potential for the transfer of nucleic acids into cells. Such is the case of the Replicating Episomal Vectors, that have been engineered and used for the study of gene expression and in gene therapy applications. In some embodiments, ectopic expression is achieved through activating the expression of the endogenously encoded proteins by activation through exogenously introduced elements, e.g., Tal effector-like nucleases (TALENS), zing finger nucleases (ZFN), Cas9 molecules, etc. In some embodiments, the transposons are, for example, Piggy bac or Sleeping Beauty transposons. In further embodiments, in vitro transcribed (IVT) mRNA encoding the factors is delivered to the cells.

In some embodiments, factors that are receptor ligands, e.g., growth factors, cytokines, etc. are expressed by the cells of the instant disclosure. In such embodiments, the receptor ligands may signal in an autocrine, or paracrine manner in vitro. However, in some embodiments, cytokine or growth factor receptors are also exogenously expressed in the cell. In such embodiments, the receptors may be wild type, that is, not comprising any mutations that alter their function as compared to the standard receptor known in the art. In other embodiments, receptors comprise mutations that alter their function are contemplated. In one such example, mutations may render the receptors as “constitutively active”, meaning that the receptors signal in the absence of their ligand. Several such constitutively active receptors are known in the art, for example, mutant FGF receptor. See, for example, Brewer et al. “Genetic insights into the mechanisms of Fgf signaling”, Genes Dev. 2016 Apr. 1; 30(7): 751-771, which is incorporated by reference herein. Furthermore, Overexpression or mutation of Epidermal growth factor receptor (EGFR) can also lead to constitutive expression. See, for example, Chakraborty et al. “Constitutive and ligand-induced EGFR signaling triggers distinct and mutually exclusive downstream signalling networks”, Nature Communications volume 5, Article number: 5811 (2014); and Holland et al. “A constitutively active epidermal growth factor receptor cooperates with disruption of G1 cell-cycle arrest pathways to induce glioma-like lesions in mice”, Genes & Dev. 1998. 12: 3675-3685, which are incorporated by reference herein in their entireties including, but not limited to the sequences and mutations necessary for constitutive activation. In some embodiments, EGFR mutations involve deletion of most of the extracytoplasmic domain of the receptor, resulting in a hybrid mRNA between new sequences and the truncated EGFR sequence. See, for example, Wong A. J. et al. “Structural alterations of the epidermal growth factor receptor gene in human gliomas”, PNAS Apr. 1, 1992 89 (7) 2965-2969, which is incorporated by reference herein in its entirety. In some embodiments, constitutively active PDGF is contemplated. Various mutations in PDGF result in constitutively active signaling, e.g., mutations that effect two regions within the receptor, the autoinhibitory juxtamembrane region (exon 12 mutations) and the kinase domain itself (exon 14 and exon 18 mutations). Whereas exon 14 mutations affect the upper lobe of the kinase domain, exon 18 mutations are located within the activation loop. See, for example, Bahlawane et al. “Constitutive activation of oncogenic PDGFRα-mutant proteins occurring in GIST patients induces receptor mislocalisation and alters PDGFRα signalling characteristics”, Cell Commun Signal. 2015; 13: 21; and Heinrich, M. C. et al. “PDGFRA activating mutations in gastrointestinal stromal tumors”, Science. 2003 Jan. 31; 299(5607):708-10, which are incorporated by reference herein regarding the constitutive activation and sequences required for such activation. In some embodiments, constitutive insulin receptor is contemplated for use as a factor. See, for example, Yamada et al. “Substitution of the insulin receptor transmembrane domain with the c-neu/erbB2 transmembrane domain constitutively activates the insulin receptor kinase in vitro”, Journal Biol Chem, Volume 267, Issue 18, 25 Jun. 1992, Pages 12452-12461. In some embodiments, constitutively active FGFR as a factor is contemplated. See, for example, Rutland P et al. “Identical mutations in the FGFR2 gene cause both Pfeiffer and Crouzon syndrome phenotypes”, Nature Genetics volume 9, pages 173-176 (1995), which is incorporated by reference herein. Transition of T to C at nucleotide 1036 has been reported in human subjects. The resulting Cys342Arg mutation has previously been observed in a single case of Crouzon syndrome. Other mutation are a G to A transition at nucleotide 1037-Tyr for Cys, which has previously been reported in three cases of Crouzon syndrome. Other suitable mutations of FGFR2 are an A to C transversion at nucleotide 1033, changing Thr to Pro at position 341, adjacent to the Cys 342 residue. Furthermore, a mutation that replaces the cysteine at position 342 with tyrosine, thus disrupting the formation of the third immunoglobulin (Ig)-like loop in the extracellular portion of the receptor and results in constitutive activation is contemplated as a factor for use in the methods of the current disclosure. See, for example, Mangasarian et al. “Mutation associated with Crouzon syndrome causes ligand-independent dimerization and activation of FGF receptor-2”, Journal of Cell. Phys. Vol. 172, Issue 1, July 1997, pp. 117-125, which is incorporated by reference herein.

In some embodiments of the current disclosure, the population of cells is grown for the purpose of producing comestible or edible products, i.e., lab-grown meat. Therefore, the culture conditions for producing such products must be carefully controlled to ensure the safety and wholesomeness of the resulting product. Further, the culture conditions may be amenable to industrial size production and GMP. For example, all culture materials, vessels, growth factors, media, etc. must be carefully selected and controlled to prevent the growth of pathogenic organisms or introduce toxins or pollutants into the product.

In some embodiments, the cells are propagated from primary cells. The term “primary cells” refers to cells taken directly from living tissue (e.g. muscle or fat tissue of an animal or a biopsy material) and established for growth in vitro. Primary cells are contemplated to be, without limitation, fibroblasts, adipocytes, hepatocytes, etc. Primary cells usually have undergone very few population doublings in culture and are therefore more representative of the main functional component of the tissue from which they are derived in comparison to continuous (tumor or artificially immortalized) cell lines. In some embodiments, the cells are immortalized precursor cells, which are immortalized through various methods, e.g., TERT or CDK4 expression. The primary cells may be derived from an animal source described herein. The cells may be from animal source including, without limitation, from bovine, avian (e.g., chicken, quail), porcine, seafood, or murine sources. The cells may also be derived from seafood such as fish (e.g., salmon, tuna, tilapia, perch, mackerel, cod, sardine, trout, etc.), shellfish (e.g., clams, mussels, and oysters); crustaceans (e.g., lobsters, shrimp, prawns, and crayfish), echinoderms (e.g., sea urchins and sea cucumbers), and insects. In another embodiment, the cells are propagated from stem cells. In some embodiments, the stem cells are derived from an animal. In some embodiments, the stem cells are induced pluripotent stem cells that are derived from animal cells (e.g., muscle cells, fat cells, connective tissue). Methods of producing induced pluripotent stem cells are known in the art.

In other embodiments, the methods comprise using a mixture of cells, e.g., “feeder cells” and target cells in culture. As used herein, “feeder cells” are modified cells that produce factors that aid in, for example, the culturing, differentiation or overall production of “target cells”. In some embodiments, the feeder cells produce the factors in a culture system such that the target cells are not genetically modified but benefit from the factors secreted by the feeder cells which are genetically modified. In some embodiments, the feeder cells and the target cells are separated by a permeable or semi-permeable membrane. In some embodiments, the factors secreted by the feeder cells are able to cross the permeable or semi-permeable membrane and induce signaling on the target cells without contamination of the target cells with the feeder cells in the final product of the method.

The use of serum as a source of growth factors and other vital proteins is a major cost in the generation of cultured meat. Further, given that serum is derived from bovine and other animal sources and cannot be fully characterized, it provides obstacles for standardization and GMP protocol development. Therefore, methods and compositions to reduce or eliminate the costs associated with using animal serum to culture cells for comestible meat products would greatly increase the production appeal and market appeal of such products. In the instant disclosure, cells and methods to generate “self-sufficient” modified cells are provided. In some embodiments, self-sufficient cells exogenously express two or more factors selected from Table 1. In some embodiments, cells express three or more factors from Table 1. In some embodiments, cells express four or more factors from Table 1.

TABLE 1
List of growth factors for use in the methods
and compositions disclosed herein.
Protein name

Neuregulin (NRG)
Insulin (INS)
Serotransferrin (TF)
Fibroblast growth factor 1 (FGF1)
Fibroblast growth factor receptor 1 (FGFR1)
Fibroblast growth factor receptor 2 (FGFR2)
Transforming growth factor beta 1 (TGFb1)
Transforming growth factor beta 3 (TGFb3)
Transforming growth factor beta receptor 2 (TGFBR2)
Insulin-like growth factor (IGF)
Insulin-like growth factor receptor 1 (IGF1R)
Platelet-derived growth factor (PDGF)
Platelet-derived growth factor receptor alpha subunit (PDGFRa)
Platelet-derived growth factor receptor beta subunit (PDGFRb)
Cardiotropin (CT1)
Leukemia inhibitory factor receptor subunit alpha (LIF1Ra)
Hepatocyte growth factor (HGF)
Hepatocyte growth factor receptor (HGFR)
Epidermal growth factor (EGF)
Epidermal growth factor receptor (EGFR)
Pigment-epithelium derived growth factor (PEDF)
Somatotropin (growth hormone, GH)
Somatotropin receptor (growth hormone receptor, GHR)
Interleukin 6 (IL-6)
Interleukin 6 receptor (IL-6R)
Leukemia inhibitory factor (LIF)
Tumor necrosis factor alpha (TNFa)
Vascular endothelial growth factor (VEGF)
Vascular endothelial growth factor receptor (VEGFR)
IGF-1_LR3
Transforming growth factor beta receptor 1 (TGFbR1)
Insulin receptor (IR)
Adipose triglyceride lipase (ATGL, receptor for PEDF)
Mechanogrowth factor (MGF, splice variant of IGF)
Transforming growth factor beta 1 (TGFb1)
Transforming growth factor beta 2 (TGFb2)

In order to exogenously express factors, the disclosed methods require delivering or expressing factors to enable cells to become self-sufficient. As used herein, delivering or grammatical variations thereof refer to the process of contacting the cultured cells with the delivered agent such that the agent has the intended effect on the target cell. The term expressing as used herein refers to the cells ability to produce the intended factor (e.g., protein) in the cells such that the cell is self-sufficient and does not require endogenous factors (e.g., proteins) for tissue culture growth in vitro.

In some embodiments, expression of the two or more factors is achieved by genetically modifying the cell to contain a polynucleotide(s) that encode and are capable of expressing the two or more factors. As used herein, genetic modification refers to changes in the nucleotide sequence of the genome of a cell or organism, either in a coding, i.e., a region which is transcribed into mRNA, or a non-coding region of the genome that results in a non-naturally occurring cells that is capable of expression of the factors described herein.

In some embodiments, the polynucleotides of the present disclosure may be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation (Davis, L., Dibner, M., Battey, I., 1986 “Basic Methods in Molecular Biology”). Other methods of transformation include for example, lithium acetate transformation and electroporation (see, e.g., Gietz et al., Nucleic Acids Res. 27:69-74 (1992); Ito et al., J. Bacterol. 153:163-168 (1983); and Becker and Guarente, Methods in Enzymology 194:182-187 (1991)).

In some embodiments, the present disclosure teaches methods for introducing exogenous protein, RNA, and DNA into a cell. Various methods for achieving this have been described previously including direct transfection of protein/RNA/DNA or DNA transformation followed by intracellular expression of RNA and protein (Dicarlo, J. E. et al. “Genome engineering in Saccharomyces cerevisiae using CRISPR-Cas systems.” Nucleic Acids Res (2013). doi:10.1093/nar/gkt135; Ren, Z. J., Baumann, R. G. & Black, L. W. “Cloning of linear DNAs in vivo by overexpressed T4 DNA ligase: construction of a T4 phage hoc gene display vector.” Gene 195, 303-311 (1997); Lin, S., Staahl, B. T., Alla, R. K. & Doudna, J. A. “Enhanced homology-directed human genome engineering by controlled timing of CRISPR/Cas9 delivery.” Elife 3, e04766 (2014)).

In some embodiments, the polynucleotide is a nucleic acid construct comprising a endogenous promoter linked to the polynucleotide sequence encoding the factor(s). In some embodiments, the construct is a vector. As used herein, the term “vector” refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors” (or simply, “vectors”). The term vector encompasses “plasmids”, the most commonly used form of vector. Plasmids are circular double-stranded DNA loops into which additional DNA segments, e.g., factor, may be ligated. However, other forms of expression vectors, such as viral vectors (e.g., replication defective retroviruses, adenoviruses and adena-associated viruses), may also be used with the present invention. Certain vectors are capable of autonomous replication in a host cell into which they are introduced (e.g., bacterial vectors having a bacterial origin of replication and episomal mammalian vectors). Other vectors may be integrated into the genome of a host cell upon introduction into the host cell, and are thereby replicated along with the host genome. In one embodiment, the vectors comprise viral vectors that use viral machinery to carry the peptide to be expressed in a host cell.

In some embodiments, the vectors of the present invention further comprise heterologous nucleic acid backbone sequence. As used herein, “heterologous nucleic acid sequence” refers to a non-human nucleic acid sequence, for example, a bacterial, viral, or other non-human nucleic acid sequence that is not naturally found in a human. Heterologous backbone sequences may be necessary for propagation of the vector and/or expression of the encoded peptide. Many commonly used expression vectors and plasmids contain non-human nucleic acid sequences, including, for example, CMV promoters.

As used herein, “promoter” refers to a region of DNA where transcription of a gene is initiated. Promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

In some embodiments, muscle-cell specific promoters are used to express the factors in cultured muscle cells. See, for example, Wang et al. “Construction and analysis of compact muscle-specific promoters for AAV vectors”, Gene Therapy volume 15, pages 1489-1499 (2008); Liu et al. “Synthetic promoter for efficient and muscle-specific expression of exogenous genes”, Plasmid, Volume 106, November 2019, 102441; and Sarcar et al. “Next-generation muscle-directed gene therapy by in silico vector design”, Nature Communications volume 10, Article number: 492 (2019), which are incorporated by reference herein.

Suitable promoters for the practice of the present invention include, without limitation, constitutive, inducible, temporally regulated, developmentally regulated, chemically regulated, physically regulated (e.g., light regulated or temperature-regulated), tissue-preferred, and tissue-specific promoters. Suitable promoters include “heterologous promoters”, a term that refers to any promoter that is not naturally associated with a polynucleotide to which it is operably connected. In mammalian cells, typical promoters include, without limitation, promoters for Rous sarcoma virus (RSV), human immunodeficiency virus (HIV-1), cytomegalovirus (CMV), SV40 virus, and the like as well as the translational elongation factor EF-1α promoter or ubiquitin promoter. Those of skill in the art are familiar with a wide variety of additional promoters for use in various cell types. In some embodiments, the promoters are, without limitation, CMV, EF1a, SV40, PGK1, Ubc, Human beta actin, CAG, TRE, UAS, Ac5, and Polyhedrin promoters.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e.g., beta actin promoter (Ng, 1989; Quitsche et al., 1989), GADPH promoter (Alexander et al., 1988, Ercolani et al., 1988), metallothionein promoter (Karin et al., 1989; Richards et al., 1984); and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). A specific example could be a phosphoglycerate kinase (PGK) promoter.

“Enhancer” refers to cis-regulatory elements in the genome that cooperate with promoters to control target gene transcription. Unlike promoters, enhancers are not necessarily adjacent to target genes and can exert their functions regardless of enhancer orientations, positions and spatial segregations from target genes. Therefore, one of skill in the art must select appropriate promoters and, in some embodiments, enhancers to drive expression of proteins encoded on the delivered DNA molecule. See, for example, Meersseman C. et al. “Genetic variability of the activity of bidirectional promoters: a pilot study in bovine muscle”, DNA Research, Volume 24, Issue 3, June 2017, Pages 221-233, incorporated herein by reference, for potential bi-directional promoters active in bovine muscle. See, for example, Kern C. et al. “Functional annotations of three domestic animal genomes provide vital resources for comparative and agricultural research”, Nature Communications volume 12, Article number: 1821 (2021), incorporated herein by reference, for enhancers found in the muscle of chickens, pigs, and cows.

Exogenous expression molecules (polynucleotides) for use the disclosed methods may include one or more externally inducible transcriptional regulatory elements for inducible expression of the one or more transient immortalization factors. For example, polynucleotides useful in the invention may comprise an inducible promoter, such as a promoter that includes a tetracycline response element. In some aspects, the polynucleotide comprises a gene delivery system. Many gene delivery systems are known to those of ordinary skill in the art, and non-limiting examples of useful gene delivery systems include a viral gene delivery system, an episomal gene delivery system, an mRNA delivery system, or a protein delivery system. A viral gene delivery system useful in the invention may be an RNA-based or DNA-based viral vector. An episomal gene delivery system useful in the invention may be a plasmid, an Epstein-Barr virus (EBV)-based episomal vector, a yeast-based vector, a simian virus 40 (SV40)-based episomal vector, a bovine papilloma virus (BPV)-based vector, or the like.

Thus, in some embodiments, the methods of the current disclosure comprise introducing a polynucleotide to a population of cells that comprises a promoter that is inducible by addition or removal of another “induction factor” or “inducible factor”. In some embodiments, the induction factor is tetracycline or the analogue doxycycline. Other suitable induction factors are known in the art including, for example, cumate inducible, rapamycin inducible, FKCsA inducible, Abcisic acid inducible, tamoxifen inducible, blue-light inducible promoters and riboswitches.

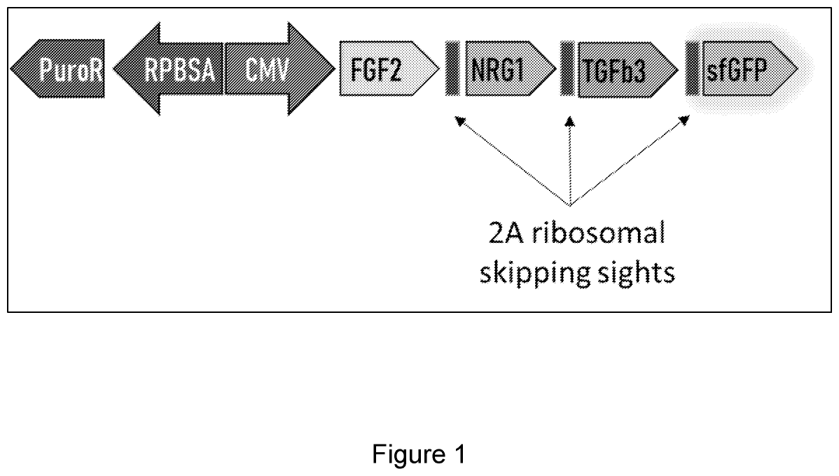

In some embodiments, the selected factors disclosed herein are under the control of inducible promoters. In some embodiments, the inducible promoter is a tetracycline inducible promoter (TetON or TetOFF). An exemplary Tet-responsive promoter is described in WO 04/056964A2 (incorporated herein by reference). See, for example, FIG. 1 of WO 04/056964A2. In one construct, a Tet operator sequence (TetOp) is inserted into the promoter region of the vector encoding the disclosed factors. TetOp is preferably inserted upstream of the transcription initiation site, upstream or downstream from the TATA box. In some embodiments, the TetOp is immediately adjacent to the TATA box. The expression of the target protein encoding sequence is thus under the control of tetracycline (or its derivative doxycycline, or any other tetracycline analogue). Addition of tetracycline or Dox relieves repression of the promoter by a tetracycline repressor that the host cells are also engineered to express. Thus, in such embodiments, the inducible factor is tetracycline.

In the TetOFF system, a different tet transactivator protein is expressed in the tetOFF host cell. The difference is that Tet/Dox, when bind to an activator protein, is now required for transcriptional activation. Thus, such host cells expressing the activator will only activate the transcription of an shRNA encoding sequence from a TetOFF promoter at the presence of Tet or Dox.

In some embodiments, the selected factors are under the control of a cumate-inducible promoter. See U.S. patent Ser. No. 10/135,362, which is incorporated by reference herein. Thus, in such embodiments, the inducible factor is cumate, or other similar compounds. Other suitable inducible promoter systems are known in the art and can be found in, for example, Kallunki et al. Cells. 2019 August; 8(8): 796, which is incorporated by reference herein. Additional inducible promoter systems include rapamycin, abscisic acid and FK506 binding protein 12 based inducible promoter systems.

Protein and nucleic acid sequence identities are evaluated using the Basic Local Alignment Search Tool (“BLAST”) which is well known in the art (Karlin and Altschul, 1990, Proc. Natl. Acad. Sci. USA 87: 2267-2268; Altschul et al., 1997, Nucl. Acids Res. 25: 3389-3402). The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. Preferably, the statistical significance of a high-scoring segment pair is evaluated using the statistical significance formula (Karlin and Altschul, 1990), the disclosure of which is incorporated by reference in its entirety. The BLAST programs can be used with the default parameters or with modified parameters provided by the user.

“Percentage of sequence identity” or “percent similarity” is determined by comparing two optimally aligned sequences over a comparison window, wherein the portion of the polynucleotide or peptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” or “substantial similarity” of polynucleotide or peptide sequences means that a polynucleotide or peptide comprises a sequence that has at least 75% sequence identity. Alternatively, percent identity can be any integer from 75% to 100%. More preferred embodiments include at least: 75%, 80%, 85%, 86%, 87%, 88%, 89%, 90%, 91%, 92%, 93%, 94%, 95%, 96%, 97%, 98% or 99% compared to a reference sequence using the programs described herein; preferably BLAST using standard parameters, as described. These values can be appropriately adjusted to determine corresponding identity of proteins encoded by two nucleotide sequences by taking into account codon degeneracy, amino acid similarity, reading frame positioning and the like.

“Substantial identity” of amino acid sequences for purposes of this invention normally means polypeptide sequence identity of at least 75%. Preferred percent identity of polypeptides can be any integer from 75% to 100%. More preferred embodiments include at least 75%, 80%, 85%, 86%, 87%, 88%, 89%, 90%, 91%, 92%, 93%, 94%, 95%, 96%, 97%, 98%, 98.7%, or 99%.

In some embodiments, “sleeping beauty” transposons are used to introduce polynucleotides into target cells. See, for example, Sharma et al: “Efficient Sleeping Beauty DNA Transposition From DNA Mini circles”, Molecular Therapy-Nucleic Acids, vol. 2, No. 2, Feb. 1, 2013, p. e74; Kacherovsky et al., “Combination of Sleeping Beauty transposition and chemically induced dimerization selection for robust production of engineered cells.” Nucleic Acids Research, vol. 40, No. 11, Jun. 1, 2012, pp. e85-e85; Kacherovsky et al: “Multiplexed 1-16 gene transfer to a human T-cell line by combining Sleeping Beauty transposon system with methotrexate selection”, Biotechnology and Bioengineering, vol. 112, No. 7, Jul. 23, 2015 (Jul. 23, 2015), pp. 1429-1436; Kay et al, “A robust system for production of minicircle DNA vectors”, Nature Biotechnology, vol. 28, No. 12, Nov. 21, 2010, pp. 1287-1289, incorporated by reference herein.

As the expression of multiple different factors in cells is required for the generation of the modified cells of the instant disclosure, ribosomal skipping sequences are used to generate several functional proteins from a single transcript. Suitably, polynucleotides encoding the factors comprise any ribosomal skipping sequence that is known in the art. In some embodiments, the ribosomal skipping sequences are 2A oligonucleotide sequences, e.g., T2A sequence (GSGEGRGSLLTCGDVEENPGP, SEQ ID NO: 66) or P2A sequence (GSGATNFSLLKQAGDVEENPGP, SEQ ID NO: 67) or combinations thereof. In some embodiments, the polynucleotides comprise internal ribosome entry sequences (IRES).

In some embodiments, the modified cells of the instant disclosure express two or more factors from Table 1, alternatively three or more factors selected from Table 1, alternatively 4 or more factors selected from Table 1, alternatively five or more factors selected from Table 1. The factors contemplated in Table 1 may be combined in a number of different combinations to provide modified cells that has superior growth properties under minimal defined media conditions as described herein. In some embodiment, the combination of the two or more factors of Table 1 are capable of increasing the growth of the modified cells in vitro in minimal defined media as compared to unmodified cells in the same minimal defined media.

In one example, the two or more factors expressed by the modified cells are selected from fibroblast growth factor (FGF), transforming growth factor (TGF), neuregulin (NRG), insulin, insulin-like growth factor (IGF), FGF-receptor (FGF-R), insulin, albumin, and combinations thereof. For example, in some embodiments, the modified cells can express (a) FGF-2, receptor FGFR1 or combination thereof; (b) transferrin; (c) insulin, insulin-like growth factor (IGF) or combination thereof; (d) recombinant albumin; (e) NRG, NRG receptor or combinations thereof; (f) TGF, TGF receptor or combinations thereof; (g) combinations of any of (a)-(f). For example, the modified cells may express any suitable combinations of factors from (a)-(f), such as, but not limited to, (a), (a) and (b), (a) and (c), (a) and (d), (a) an (e), (a) and (f), (b) and (c), (b) and (d), (b) and (e), (b) and (f), (c) and (d), (c) and (e), (c) and (f), (d) and (e), (d) and (f), (e) and (f), (a) and (b) and (c), (a) and (b) and (The d), etc. In one suitable embodiment, the modified non-human cell express factors FGF-2, NRG1, and TGF-beta3. In some embodiments, the cell further expresses albumin. In some further embodiments, the cell expresses transferrin.

### Minimal Media

In some embodiments, the cells of the instant disclosure are capable of growing in “minimal media”. The term minimal media is used interchangeable with the term “minimal defined media” and refers to media in which are the components are able to be identified and chemically defined and suitable does not contain any animal serum. As used herein, minimal media refers to media containing a carbon source (e.g., glucose), amino acids, salts, minerals, trace nutrients (e.g., vitamins), and optionally recombinant albumin or crude plant hydrolysates (e.g., to replace albumin if not endogenously produced). In a preferred example, the minimal media does not contain any growth factors. In another preferred example, the minimal media does not contain any recombinant exogenous proteins (e.g., media consists of carbon source (e.g., glucose), amino acids, salts, minerals, trace nutrients (e.g., vitamins)). Suitable chemically defined and minimal media are known in the art, for example, B8 medium (see, for example, Kuo et al. “Negligible-Cost and Weekend-Free Chemically Defined Human iPSC Culture”, Stem Cell Report. 2020 Feb. 11; 14(2):256-270, which is incorporated by reference) Beefy-9 (see, for example, Sout A. J. et al. “Simple and effective serum-free medium for sustained expansion of bovine satellite cells for cell cultured meat”, doi.org/10.1101/2021.05.28.446057, which is incorporated by reference herein), essential 8 and other serum-free media with growth factors removed (see, for example, Das, M et al. “Developing a Novel Serum-Free Cell Culture Model of Skeletal Muscle Differentiation by Systematically Studying the Role of Different Growth Factors in Myotube Formation”, In Vitro Cell Dev Biol Anim. 2009 July-August; 45(7): 378-387, which is incorporated by reference herein).

### Exemplary Factors for Use in the Compositions and Methods of the Current Disclosure

As described herein, the modified cells and meat product described herein comprise two or more factors in Table 1. Table 1 comprises growth factors and their receptors, cytokines, and other protein factors that have been found to play a role in the ability to grow and propagate cells in in vitro culture. Some of the factors are described in more detail below and exemplary sequences of the factors for use in the present invention are provided for exemplary purposes only in Table 2, but the present invention is not limited thereto. Other suitable sequences and modifications may be readily ascertained and determined by one skilled in the art.

One suitable factor for practicing the invention is neuregulin (NRG). Neuregulin has been shown to have diverse functions in the development of the nervous system and play multiple essential roles in vertebrate embryogenesis including: cardiac development, Schwann cell and oligodendrocyte differentiation, some aspects of neuronal development, as well as the formation of neuromuscular synapses. Bovine neuregulin has the sequence SEQ ID NO: 1. Tilapia (Oreochromis niloticus) neuregulin has the sequence SEQ ID NO: 2.

Another suitable factor for practicing the invention is insulin. Insulin regulates the cellular uptake of glucose. Bovine insulin has the sequence SEQ ID NO: 3, Tilapia insulin has the sequence SEQ ID NO: 4.

Another suitable factor for practice of the invention is serotransferrin or transferrin. Serotransferrin is an iron binding transport protein which can bind two Fe(3+) ions in association with the binding of an anion, usually bicarbonate. Bovine serotransferrin has the sequence SEQ ID NO: 5. Tilapia serotransferrin has the sequence SEQ ID NO: 6.

Another suitable factor for practice of the invention is fibroblast growth factor (FGF). FGF stimulates blood vessel growth and is an important player in wound healing. Bovine FGF1 has the sequence SEQ ID NO: 7. Tilapia FGF1 has the sequence SEQ ID NO: 8. Fibroblast growth factor receptor 2 (FGFR2) Bovine FGFR2 has the sequence SEQ ID NO: 11.

Another suitable factor for practice of the invention is transforming growth factor (TGF). One suitable TGF is TGF3beta (TGF3b) is a multifunctional protein that regulates embryogenesis and cell differentiation and is required in various processes such as secondary palate development. Bovine TGF3b has the sequence SEQ ID NO: 15. Tilapia TGF3b has the sequence SEQ ID NO: 16.

Another suitable factor for practice of the invention is the receptor for fibroblast growth factor-1, e.g. FGF receptor 1 (FGFR1). FGFR1 is membrane receptor involved in many cellular processes including proliferation and migration. Bovine FGFR1 has the sequence SEQ ID NO: 9. Tilapia FGFR hast the sequence SEQ ID NO: 10. In some embodiments, the modified cell of the present disclosure can express FGF-1, FGFR1, or both FGF1 and FGFR1 in the modified cell. In some embodiments, the modified cell can express a constitutively active FGFR1 receptor which is actively on even without binding of FGF, and therefore removed the need for modifying the cell to express FGF-1. See, for example, Rutland et al. supra, and Mangasarian et al., supra.

Another suitable factor for practice of the invention is insulin-like growth factor 1 (IGF-1). IGF-1 is a hormone that helps promote bone and tissue growth. Bovine IGF-1 has the sequence SEQ ID NO: 18. Tilapia IGF-1 has the sequence SEQ ID NO: 19. Insulin can also be a factor expreseed by the modified cells of the present invention. In some embodiments, the cell may express both IGF-1 and insulin. In some aspects, for example, delivery of different combinations of factors are disclosed herein. In one embodiment, mRNA is delivered to the population of cells. In another embodiment, nucleic acid sequences (e.g., DNA) encoding the factors described herein are used.

The invention of the current disclosure provides, in some embodiments, methods of generating self-sufficient modified cells. Thus, in some embodiments, factors capable of CRISPR interference (CRISPRi) and/or CRISPR activation (CRISPRa) are used in the methods and compositions of the current disclosure to allow expression of the two or more factors. In some embodiments, the present disclosure teaches methods of modulating the expression of host cell genes via CRISPRi (CRISPR interference) and CRISPRa (CRISPR activation) technologies. In some embodiments, the presently disclosed technologies utilize catalytically inactivated (i.e., nuclease-deactivated) CRISPR endonucleases that have been mutated to no longer generate double DNA stranded breaks, but which are still able to bind to DNA target sites through their corresponding guide RNAs. In some embodiments, the present disclosure refers to these catalytically inactivated CRISPR enzymes as “dead CRISPR”, or “dCRISPR” enzymes. The “dead” modifier may also be used in reference to specific CRISPR enzymes, such as dead Cas9 (dCas9), or dead Cpf1 (dCpf1).

In some embodiments, CRISPR or other suitable genome editing methods are used to modify the FGFR such that cysteine at position 342 is replaced with tyrosine, thus disrupting the formation of the third immunoglobulin (Ig)-like loop in the extracellular portion of the receptor and conferring constative activity to the receptor. This constitutive FGFR can be expressed in the cells described herein.

dCRISPR enzymes function by recruiting the catalytically inactivated dCRISPR enzyme to a target DNA sequence via a guide RNA, thereby permitting the dCRISPR enzyme to interact with the host cell's transcriptional machinery for a particular gene.

In some embodiments, The CRISPRi methods of the present disclosure utilize dCRISPR enzymes to occupy target DNA sequences necessary for transcription, thus blocking the transcription of the targeted gene (L. S. Qi et al., “Repurposing CRISPR as an RNA-Guided Platform for Sequence-Specific Control of Gene Expression.” Cell. 152, 1173-1183 (2013); see also L. A. Gilbert et al., “CRISPR-Mediated Modular RNA-Guided Regulation of Transcription in Eukaryotes.” Cell. 154, 442-451 (2013)). In other embodiments, the CRISPRi methods of the present disclosure utilize dCRISPR enzymes translationally fused, or otherwise tethered to one or more transcriptional repression domains, or alternatively utilize modified guide RNAs capable of recruiting transcriptional repression domains to the target site (e.g., tethered via aptamers, as discussed below).

In some embodiments, the CRISPRa methods of the present disclosure employ dCRISPR enzymes translationally fused or otherwise tethered to different transcriptional activation domains, which can be directed to promoter regions by guide RNAs. (See A. W. Cheng et al., “Multiplexed activation of endogenous genes by CRISPR-on, an RNA-guided transcriptional activator system.” Cell Res. 23, 1163-1171 (2013); see also L. A. Gilbert et al., “Genome-Scale CRISPR-Mediated Control of Gene Repression and Activation.” Cell. 159, 647-661 (2014)). In other embodiments, the CRISPRa methods of the present disclosure utilize modified guide RNAs that recruit additional transcriptional activation domains to upregulate expression of the target gene (e.g., tethered via aptamers, as discussed below). In some embodiments, CRISPRa is used to activate expression of endogenous factors, for example, those listed in Table 1.

In yet other embodiments, the presently disclosed invention also envisions exploiting dCRISPR enzymes and guide RNAs to recruit other regulatory factors to target DNA sites. In addition to recruiting transcriptional repressor or activation domains, as discussed above, the dCRISPR enzymes and guide RNAs of the present disclosure can be modified so as to recruit proteins with activities ranging from DNA methylation, chromatin remodelers, ubiquitination, sumoylation. Thus, in some embodiments, the dCRISPR enzymes and guide RNAs of the present disclosure can be modified to recruit factors with methyltransferase activity, demethylase activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, kinase activity, phosphatase activity, ubiquitin ligase activity, deubiquitinating activity, adenylation activity, deadenylation activity, sumoylating activity, desumoylating activity, ribosylation activity, deribosylation activity, myristoylation activity, remodelling activity, protease activity, oxidoreductase activity, transferase activity, hydrolase activity, lyase activity, isomerase activity, synthase activity, synthetase activity, demyristoylation activity, cytidine deaminase activity and any combinations thereof

In other embodiments, the dCRISPR enzymes and guide RNAs of the present disclosure can be modified to recruit one or more marker genes/composition, such as fluorescent proteins, gold particles, radioactive isotopes, GUS enzymes, or other known biological or synthetic compositions capable of being detected. This last embodiment would permit researchers to tag and track regions of a host cell's genome. As used herein, the term “cis regulatory factors” refers to any of the biological or synthetic compositions that can be recruited by the dCRISPR or guide RNAs of the present disclosure.

In some embodiments, the dCRISPR enzyme and the transcriptional modulator domain are linked via a peptide linker. A peptide linker sequence may be employed to separate the first and the second peptide components by a distance sufficient to ensure that each peptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional regions on the first and second peptides; and (3) the lack of hydrophobic or charged residues that might react with the peptide functional regions. In certain embodiments, the peptide linker sequences contain Gly, Asn and Ser residues. Other near neutral amino acids, such as Thr and Ala may also be used in the linker sequence.

In some embodiments, the present disclosure teaches the use of protein-protein interaction domains to tether the transcriptional modulator domains to the dCRISPR. Thus in some embodiments, the sequence of the dCRISPR enzyme is translationally fused to a first protein-protein interaction domain (PP1) capable of dimerizing with a second protein-protein interaction domain (PP2) that is translationally fused to the transcriptional modulator (or other cis regulatory factor). When expressed, each of the dCRISPR-PP1 and the PP2-Transcriptional Modulator will dimerize, thus recruiting the transcriptional modulator to the DNA target site. Persons having skill in the art will be aware of methods of using naturally occurring, or synthetic protein-protein interaction domains to create in-vivo dimers. (See Giescke et al., 2006 “Synthetic protein-protein interaction domains created by shuffling Cys2His2 zinc-fingers.” Mol Syst Biol 2: 2006.0011).

In other embodiments, the present disclosure also teaches modified guide RNAs with RNA aptamers capable of recruiting one or more cis regulatory factors. The RNA aptamers of the present disclosure may be operably linked to the 5′ or 3′ end of a guide RNA, and are designed so as to not affect dCRISPR binding to a DNA target site. Instead, the RNA aptamers provide an additional tether from which to recruit one or more cis regulatory factors, such as transcriptional modulators.

In some embodiments, the present disclosure teaches customized RNA aptamers designed to directly interact with one or more cis regulatory factors. In other embodiments, the present disclosure teaches use of known aptamers targeting specific sequences. Thus, in some embodiments, the present disclosure envisions guide RNAs with validated RNA aptamers, which then bind to their natural targets, which are in turn translationally fused to one or more cis regulatory factor (i.e., guide_RNA-Aptamer-Aptamer_Target-Cis_Regulatory_Factor). In some embodiments, guide RNAs that incorporate RNA aptamers to tether cis regulatory factors are referred to as scaffold RNAs (scRNAs). (Zalatan J G, et al. “Engineering complex synthetic transcriptional programs with CRISPR RNA scaffolds.” Cell. 2015; 160:339-350). The scRNAs are designed by extending the guide RNA sequence with orthogonally acting protein-binding RNA aptamers. Each scRNA can encode information both for DNA target recognition and for recruiting a specific repressor or activator protein. By changing the DNA targeting sequence or the RNA aptamers in a modular fashion, multiple dCas9-scRNAs can simultaneously activate or repress multiple genes in the same cell

For example, an improvement, termed the synergistic activation mediator (SAM) system, was achieved by adding MS2 aptamers to a guide RNA. The MS2 aptamers were designed to recruit cognate MS2 coat protein (MCP), which were fused to p65AD and heat shock factor 1 (HSF1) (Dominguez et al., 2016 “Beyond editing; repurposing CRISPR-Cas9 for precision genome regulation and interrogation” Nat Rev Mol Cel Biol January 17(1) 5-15). The SAM technology, together with dCas9-VP64, further increased endogenous gene activation compared with dCas9-VP64 alone and was shown to activate 10 genes simultaneously. (Konermann S, et al. “Genome-scale transcriptional activation by an engineered CRISPR-Cas9 complex.” Nature. 2014; 517:583-588). Similar results may be achieved through the use of other validated aptamer-scaffold protein combinations, such as PP7 or com. (Zalatan J G, et al. “Engineering complex synthetic transcriptional programs with CRISPR RNA scaffolds.” Cell. 2015; 160:339-350).

In some embodiments, the present disclosure also envisions the use of double-sided aptamers capable of tethering a dCRISPR enzyme to one or more cis regulatory factors. The double-sided aptamers of the present disclosure function similarly to the aptamers discussed above, but are capable of binding both the dCRISPR protein, and the cis regulatory factor. In one illustrative example, the dCRISPR enzyme would be translationally fused to an MS2 coat protein domain, and the cis regulatory element (a VP16 domain) would be translationally fused to a PP7 domain. The double-sided RNA aptamer would comprise an MS2 binding domain on one end, and a PP7 binding domain on another end. Thus, in some embodiments, the double-sided aptamers of the present disclosure can would be expected to form the following generic structure: dCRISPR-Aptamer_Target-Aptamer_Side1-Aptamer_Side2-Aptamer_Target-Cis_Regulatory_Factor.

A non-limiting list of the transcriptional activation domains compatible with the presently disclosed invention include: fragments of transcription regulatory domains and fragments of domains having transcription regulation function of VP16, VP64, VP160, EBNA2, E1A, Ga14, Oaf1, Leu3, Rtg3, Pho4, Gln3, Gcn4, Gli3, Pip2, Pdr1, Pdr3, Lac9, Teal, p53, NFAT, Sp1 (e.g., Sp1a), AP-2 (e.g., Ap-2a), Sox2, MLL/ALL, E2A, CREB, ATF, FOS/JUN, HSF1, KLF2, NF-1L6, ESX, Oct1, Oct2, SMAD, CTF, HOX, Sox2, Sox4, VPR, RpoZ, or Nanog. In some embodiments the transcriptional activator is VPR (see Kiani S. et al., “Cas9 gRNA engineering for genome editing, activation and repression” Nature Methods 12, 1051-1054 (2015)).

In some embodiments, the nucleic acids encoding for the dCRISPR enzyme and/or the guide RNA are contained in one or more insert parts of a modular CRISPR construct of the present disclosure. Thus, in some embodiments, the modular CRISPR constructs of the present disclosure permit users to quickly and efficiently modify the construct to add or subtract insert parts encoding for different guide RNAs (e.g., guide RNAs targeting different genes, or encoding aptamers capable of recruiting different cis regulatory factors, as discussed above), or encoding different dCRISPR enzymes (e.g., dCas9, or dCpf1, or dCRISPR protein fusions with various cis regulatory factors, as discussed above).

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain. In some embodiments, a protein comprises a proteinaceous part, e.g., an amino acid sequence constituting a nucleic acid binding domain.

Nucleic acids, proteins, and/or other compositions described herein may be purified. As used herein, “purified” means separate from the majority of other compounds or entities, and encompasses partially purified or substantially purified. Purity may be denoted by a weight by weight measure and may be determined using a variety of analytical techniques such as but not limited to mass spectrometry, HPLC, etc.

Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The terms “nucleic acid” and “nucleic acid molecule,” as used herein, refer to a compound comprising a nucleobase and an acidic moiety, e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), etc. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning—A Laboratory Manual, Cold Spring Harbor Laboratory, Cold Spring Harbor, New York; Wetmur, 1991, Critical Review in Biochem. and Mol. Biol. 26(3/4):227-259; and Owczarzy et al., 2008, Biochemistry, 47: 5336-5353, which are incorporated herein by reference).

In some embodiments, the present disclosure teaches the use of origins of replication to maintain (i.e., continue to replicate) a plasmid in one or more species. Persons having skill in the art will be familiar with various available origin of replication sequences. Common features of origins of replications for bacterial, archael, eukaryotic, and multicellular organisms is discussed in Leonard and Mechali, “DNA replication Origins” Cold Spring Harb Perspect Biol 2013 October; 5(10).

In some embodiments, the polynucleotides of the current disclosure comprise “selection markers”. Selection markers are factors encoded by the polynucleotide that allow selection of a cell harboring the polynucleotide. In some embodiments, selection markers are, for example, fluorescent proteins or luminescent proteins. In some embodiments, selection markers are polynucleotide sequences that encode proteins that confer resistance to antibiotics. In some embodiments, polynucleotides comprise multiple selection markers. Exemplary selection markers include puromycin, blasticidin, zeocin, G418, or hygromycin resistance. Puromycin resistance is conferred by expression of the protein with SEQ ID NO: 64. Green fluorescent protein (GFP) has the sequence SEQ ID NO: 65.

In some embodiments, the current disclosure provides a meat product comprising the modified non-human cells of the current disclosure.

### Methods for Production of In Vitro Cultured Meat Product

In another aspect of the current disclosure, methods of producing a meat product in in vitro culture are provided. In some embodiments, the methods comprise: culturing a population of the modified non-human cells, wherein the cells are ectopically expressing two or more growth factors or cytokines or receptors thereof that promote cell growth, wherein the two or more factors are selected from the factors listed in Table 1, in minimal culture medium for a sufficient time to increase the number of cells, whereby the method produces a non-human animal tissue suitable for human and/or animal consumption and wherein the minimal media does not contain exogenous growth factors. In some embodiments, the minimal culture medium consists a combination of one or more of a carbon source, amino acids, salts, minerals, trace nutrients, and optionally crude plant hydrolysates or recombinant albumin. In some embodiments, the minimal culture medium consists a combination of one or more of a carbon source, amino acids, salts, minerals, trace nutrients, and optionally crude plant hydrolysates. In some embodiments, the minimal media contains no exogenous recombinant protein components. In some embodiments the method comprises: culturing a combination of two or more modified cells selected from muscle cell, stem cells, fat cell, and connective tissue cell in a culture to produce a three dimensional meat product suitable for human or animal consumption.

In another aspect of the current disclosure, methods of producing a population of modified cells for making a food product are provided. In some embodiments, the method comprises: (a) expressing two or more factors of Table 1 in cells; (b) culturing the cells of (a) in minimal medium for a sufficient time to promote growth of cells to a sufficient number to produce a food product; wherein the minimal media does not contain exogenous growth factors. In some embodiments, the number of modified cells produced in step (b) is increase at least 10% or more, 15% or more, 20% or more, 35% or more, 30% or more, 35% or more, 40% or more, 50% or more, 55% or more, 60% or more, 65% or more, 70% or more, or 100% or more relative to culturing of non-modified non-human cells in the minimal medium. Suitable precentages and amounts inbetween these amounts are contemplate, e.g., 10%, 11%, 12%, 13%, 25%, 27%, 36%, etc.

In some embodiments, the methods of the instant disclosure comprise expressing two or more factors from Table 1, alternatively three or more factors selected from Table 1, alternatively 4 or more factors selected from Table 1, alternatively five or more factors selected from Table 1. The factors contemplated in Table 1 may be combined in a number of different combinations in the disclosed methods to provide modified cells that have superior growth properties under minimal defined media conditions as described herein. In some embodiments, the combination of the two or more factors of Table 1, when used in the disclosed methods, are capable of increasing the growth of the modified cells in vitro in minimal defined media as compared to unmodified cells in the same minimal defined media. In some embodiments, the number of modified cells produced in step (b) is increase at least 10% or more, 15% or more, 20% or more, 35% or more, 30% or more, 35% or more, 40% or more, 50% or more, 55% or more, 60% or more, 65% or more, 70% or more, or 100% or more relative to culturing of non-modified non-human cells in the minimal medium.

In one example, the two or more factors of the disclosed methods are selected from fibroblast growth factor (FGF), transforming growth factor (TGF), neuregulin (NRG), insulin, insulin-like growth factor (IGF), FGF-receptor (FGF-R), insulin, albumin, and combinations thereof. For example, in some embodiments, the disclosed methods comprise expressing (a) FGF-2, receptor FGFR1 or combination thereof; (b) transferrin; (c) insulin, insulin-like growth factor (IGF) or combination thereof; (d) recombinant albumin; (e) NRG, NRG receptor or combinations thereof; (f) TGF, TGF receptor or combinations thereof; (g) combinations of any of (a)-(f). For example, the modified cells may express any suitable combinations of factors from (a)-(f), such as, but not limited to, (a), (a) and (b), (a) and (c), (a) and (d), (a) an (e), (a) and (f), (b) and (c), (b) and (d), (b) and (e), (b) and (f), (c) and (d), (c) and (e), (c) and (f), (d) and (e), (d) and (f), (e) and (f), (a) and (b) and (c), (a) and (b) and (The d), etc. In one suitable embodiment, the methods comprise expressing factors FGF-2, NRG1, and TGF-beta3. In some embodiments of the methods, the cell further expresses albumin. In some further embodiments, the cell expresses transferrin. In some embodiments, step (a) further comprises introducing one or more, two or more, or three or more exogenous vectors in the cell capable of expressing the two or more factors.

In some embodiments of the methods, the one or more exogenous vectors is a single vector capable of expressing the two or more factors, three or more factors, four or more factors or five or more factors. In some embodiments, the vector comprises ribosomal skipping sites to express the two or more factors. In some embodiments, the one or more vector constitutively expresses the two or more factors. In some embodiments, the one or more vector is and inducible vector; and the method further comprises contacting the cell with an inducible factor, wherein contact with the inducible factor induces expression of the one or more factors within the cell. In some embodiments, step (a) comprises engineering the cell via crispr/cas9 editing to either express an endogenous factor or increase the expression of the factor within the cell. In some embodiments, the cells is a muscle cell, a fat cell, a stem cell, or a connective tissue cell. In some embodiments, the cells is a bovine cell, a piscine cell, a porcine cell, or a galline cell. In some embodiments, the cells are a combination of two or more cells selected from muscle cell, a fat cell, a stem cell, or a connective tissue cell.

### Population of Cells

In another aspect of the current disclosure, a population of cells is provided. In some embodiments, the population of cells is made by the method comprising: culturing a population of the modified non-human cells, wherein the cells are ectopically expressing two or more growth factors or cytokines or receptors thereof that promote cell growth, wherein the two or more factors are selected from the factors listed in Table 1, in minimal culture medium for a sufficient time to increase the number of cells, whereby the method produces a non-human animal tissue suitable for human and/or animal consumption and wherein the minimal media does not contain exogenous growth factors. In some embodiments, the population of cells is made the method comprising: (a) expressing two or more factors of Table 1 in cells; (b) culturing the cells of (a) in minimal medium for a sufficient time to promote growth of cells to a sufficient number to produce a food product; wherein the minimal media does not contain exogenous growth factors. In some embodiments, the number of modified cells produced in step (b) is increase at least 10% or more, 15% or more, 20% or more, 35% or more, 30% or more, 35% or more, 40% or more, 50% or more, 55% or more, 60% or more, 65% or more, 70% or more, or 100% or more relative to culturing of non-modified non-human cells in the minimal medium.

### Food Product

In another aspect of the current disclosure, food products are provided. In some embodiments, the food products comprise a population of cells made by the method comprising: culturing a population of the modified non-human cells, wherein the cells are ectopically expressing two or more growth factors or cytokines or receptors thereof that promote cell growth, wherein the two or more factors are selected from the factors listed in Table 1, in minimal culture medium for a sufficient time to increase the number of cells, whereby the method produces a non-human animal tissue suitable for human and/or animal consumption and wherein the minimal media does not contain exogenous growth factors. In some embodiments, the food product comprises a population of cells made the method comprising: (a) expressing two or more factors of Table 1 in cells; (b) culturing the cells of (a) in minimal medium for a sufficient time to promote growth of cells to a sufficient number to produce a food product; wherein the minimal media does not contain exogenous growth factors. In some embodiments, the number of modified cells produced in step (b) is increase at least 10% or more, 15% or more, 20% or more, 35% or more, 30% or more, 35% or more, 40% or more, 50% or more, 55% or more, 60% or more, 65% or more, 70% or more, or 100% or more relative to culturing of non-modified non-human cells in the minimal medium. In some embodiments, the food product is a three dimensional tissue suitable for human or non-human animal consumption.

In another aspect of the current disclosure, consumable compositions are provided. In some embodiments, the compositions comprise a population of cells made by the methods described herein. In one embodiment, the composition comprises one or more target cell type made by the methods described herein. In another embodiment, the consumable composition comprising one or more populations of the non-human modified cells described herein. In one embodiment, the consumable composition is made by a method comprising: culturing a population of the modified non-human cells, wherein the cells are ectopically expressing two or more growth factors or cytokines or receptors thereof that promote cell growth, wherein the two or more factors are selected from the factors listed in Table 1, in minimal culture medium for a sufficient time to increase the number of cells, whereby the method produces a non-human animal tissue suitable for human and/or animal consumption and wherein the minimal media does not contain exogenous growth factors. In some embodiments, the compsition comprises a population of cells made the method comprising: (a) expressing two or more factors of Table 1 in cells; (b) culturing the cells of (a) in minimal medium for a sufficient time to promote growth of cells to a sufficient number to produce a food product; wherein the minimal media does not contain exogenous growth factors. In some embodiments, the number of modified cells produced in step (b) is increase at least 10% or more, 15% or more, 20% or more, 35% or more, 30% or more, 35% or more, 40% or more, 50% or more, 55% or more, 60% or more, 65% or more, 70% or more, or 100% or more relative to culturing of non-modified non-human cells in the minimal medium. In some embodiments, the composition comprises a three dimensional tissue suitable for human or non-human animal consumption.

In some embodiments, the meat product or composition produced by the methods of the invention may be intended for consumption by human beings, non-human animals, or both. In some embodiments, the cultured meat products are food products for human consumption. In other embodiments, the cultured meat products are used for animal feed such as feed for livestock, feed for aquaculture, or feed for domestic pets.

In some embodiments, the method includes culturing myoblasts in vitro or ex vivo and allowing these cells to differentiate into specific types of muscle cells such as skeletal muscle cells or smooth muscle cells.

In some embodiments, the meat product comprises muscle cells including skeletal muscle cells, smooth muscle cells and satellite cells. In some embodiments, the meat product comprises fat cells (e.g., adipocytes). In some embodiments, the meat product comprises an extra cellular matrix secreted by specialized cells (e.g., fibroblasts). In some embodiments, the meat product comprises endothelial cells or capillary endothelium formed by endothelial cells, including, but not limited to aortic endothelial cells and skeletal microvascular endothelial cells. In some embodiments, the meat product further comprises an extracellular matrix. In some embodiments, the meat product further comprises adipocytes. In some embodiments, the meat product further comprises capillaries.

The cells may be edible cells including muscle cells, fat cells, and combinations thereof. The precursor cells may be muscle precursor cells or adipoctye precursor cells. Examples of suitable cell types include, but are not limited to, satellite cells, fat cells (i.e., adipocytes), fibroblasts, myoblasts, muscle cells, precursors thereof, and combinations thereof. The cells may be derived from primary cells of suitable animals, as described herein.

The cells may be from animal source including, without limitation, from bovine, avian (e.g., chicken, quail), porcine, seafood, or murine sources. The cells may also be derived from seafood such as fish (e.g., salmon, tuna, tilapia, etc.), shellfish (e.g., clams, mussels, and oysters); crustaceans (e.g., lobsters, shrimp, prawns, and crayfish), and echinoderms (e.g., sea urchins and sea cucumbers), and insects. In some embodiments, the cells may be engineered to produce vital nutrients such as proteins and essential fatty acids.

In some aspects, media formulations may include transgenic components to drive cell growth and/or differentiation. For example, tetracycline-responsive promoters inserted into transgenic cells may be activated by including tetracycline in the culture medium, resulting in forced expression of myogenic or adipogenic genes in edible cell lines (e.g., chicken fibroblasts, bovine satellite cells, etc.).

Bovine satellite cells may be cultured in growth media (e.g., DMEM with Glutamax, and 1% antiobiotic-antimycotic) and modified to express two or more factors described herein. In some embodiments, the method comprises a differentiation step and a growth step in minimal culture medium. In one embodiment, to differentiate satellite cells into mature myotubes, cells may be cultured to confluence and triggered for differentiation by a low growth factor environment. For example, the culture medium may shift from a growth factor-rich proliferation media to a growth factor-poor differentiation media.

Bovine fat cells may also be cultured in growth media (e.g., DMEM with Glutamax, 1% antibiotic-antimycotic). To differentiate adipogenic precursor cells into mature adipocytes, cells may be cultured to a desired confluence (e.g., 75%), and the media may then be supplemented with free fatty acid solution or the cells can be modified to express at least one of the adipogenic factors found in Table 3. An exemplary free fatty acid solution may be 50 millimolar (mM) free fatty acid solutions containing elaidic acid, erucic acid, myristoleic acid, oleic acid, palmitoleic acid, phytanic acid, and pristanic acid. To verify lipid accumulation, Oil Red O (ORO) may be used to stain differentiated cells.

Growth factors that can be used in the methods and compositions of the invention include but are not limited to platelet-derived growth factors (PDGF), insulin-like growth factor (IGF-1). PDGF and IGF-1 are known to stimulate mitogenic, chemotactic and proliferate (differentiate) cellular responses. The growth factor can be, but is not limited to, one or more of the following: PDGF, e.g., PDGF AA, PDGF BB; IGF, e.g., IGF-I, IGF-II; fibroblast growth factors (FGF), e.g., acidic FGF, basic FGF, β-endothelial cell growth factor, FGF 4, FGF 5, FGF 6, FGF 7, FGF 8, and FGF 9; transforming growth factors (TGF), e.g., TGF-P1, TGF β1.2, TGF-β2, TGF-β3, TGF-β5; bone morphogenic proteins (BMP), e.g., BMP 1, BMP 2, BMP 3, BMP 4; vascular endothelial growth factors (VEGF), e.g., VEGF, placenta growth factor; epidermal growth factors (EGF), e.g., EGF, amphiregulin, betacellulin, heparin binding EGF; interleukins, e.g., IL-1, IL-2, IL-3, IL-4, IL-5, IL-6, IL-7, IL-8, IL-9, IL-10, IL-11, IL-12, IL-13, IL-14; colony stimulating factors (CSF), e.g., G-CSF, GM-CSF, M-CSF; nerve growth factor (NGF); stem cell factor; hepatocyte growth factor, and ciliary neurotrophic factor.

Various cost-effective biopolymers or complex extracts from natural sources may be used as coating materials for the surfaces of culture vessels used to culture the modified cells or used in the methods disclosed herein. In some embodiments, extracellular matrix proteins and/or chemical/synthetic coatings may be used as coatings to improve cell attachment to the culture vessel and mimic in vivo cell behavior. Other types of coating materials may include commercially available products such as, but not limited to, fibronectin, laminin, vitronectin, collagen, cadherin, elastin, hyaluronic acid, poly-D-lysine, poly-L-lysine, poly-L-ornithine, concanavalin A, and other adhesive, non-toxic chemicals. Conconavalin A, laminin, and hyaluronic acid may be obtained from animal-free origins and have been shown to enhance muscle cell attachment to various biomaterials.

The structural hierarchy and marbling of the cultured tissue construct may be tunable by changing the ratio of muscle cell fibers and fat cell fibers. Warner-Bratzler shear force test may be used to assess the texture and tenderness of the cultured tissue product.

According to the present disclosure, cultured muscle provides versatile outputs that meet target metrics pertaining to properties such as texture, thermal response upon cooking, composition, nutrition, density, alignment, composition, and marbling. This cultured meat system is cost-efficient, scalable, and generates cultured meats that mimic whole muscle.

PCT application number US2021/071171 describes methods and systems to generate whole muscle meat in culture. Accordingly, PCT application number US2021/071171, filed Aug. 12, 2021 is incorporated herein by reference in its entirety. The methods of transiently expanding a population of cells in culture may be used in the bioreactors described in PCT application number US2021/071171. Thus, the instantly disclosed cells and methods for developing cultured cell populations without genetically modifying the cells are suitable for use in the systems and methods disclosed in PCT application number US2021/071171. Suitable, the methods and systems are suitable for large scale production of the modified cells of the instant invention.

In some embodiments, the compositions comprise a mixture of cells, e.g., “feeder cells” and target cells. As used herein, “feeder cells” are cells that produce factors that aid in, for example, the culturing, differentiation or overall production of “target cells”. The target cells are the cells used to produce a cultured meat product. In some embodiments, the feeder cells produce the factors in a culture system such that the target cells are not genetically modified but benefit from the factors secreted by the feeder cells which are genetically modified. In some embodiments, the feeder cells and the target cells are separated by a permeable or semi-permeable membrane. In some embodiments, the factors secreted by the feeder cells are able to cross the permeable or semi-permeable membrane and induce signaling on the target cells without contamination of the target cells with the feeder cells in the final product. In some embodiments, a meat product comprising the target cells is produced by the methods described herein, wherein the target cells are not modified. This method further allows the target cells and feeder cells to grow in minimal culture medium without the addition of exogenous growth factors, and the ability to produce a meat product that does not contain modified cells. In some embodiments, the meat product comprises the target cells and is substantially free of feeder cells or is free of feeder cells.

As used herein the terms “ingestible” and “edible” refer to compositions which can be safely taken into the body. These compositions include those which are absorbed, and those which are not absorbed as well as those which are digestible and non-digestible. As used herein, the term “chewable” refers to a composition which can be broken/crushed into smaller pieces by chewing prior to swallowing. One skilled in the art will appreciate that a suitable edible composition may be selected according to physical properties (e.g., Young's modulus, viscosity modulus, stiffness, etc.) to a desired use (e.g., consumption by a human adult).

While the above work focuses on proliferation medium, it is possible that this same technique could be used for differentiation alone or in addition to cell growth to made edible meat products. For instance, an inducible promoter system could be used to express muscle-differentiation growth factors and/or their receptors (e.g., EGF (SEQ ID NO:68), EGF receptor (SEQ ID NO:69) and IGF 4 (SEQ ID NO:70)) when the cells are triggered to differentiate.

While the above work focuses on bovine muscle cells, this could be applicable to other species (in some instances by using gene homologues from these species) and other cell types. For example, the methods can be used for making fat cells for food production (e.g., with relevant growth factors such as those mentioned above as well as fatty acid binding protein 4 (FABP4), bone morphogenic protein 4 (BMP4, SEQ ID NO:72(Bovine), SEQ ID NO:71), peroxisome proliferator activated receptor γ (PPARγ, SEQ ID NO:73), or CCAAT enhancer binding protein alpha (CEBPA, SEQ ID NO:74)). Other potential applications of the disclosed technology include the use of preadipocytes, dedifferentiated fat cells (DFAT cells), mesenchymal stem cells or other similar cells, e.g., adipose derived stem cells.

Table 4 provides a list of genes that can be ectopically expressed to induce adipogenesis in various adipogenic precursor cells. As such, in one embodiment, the present invention provides modified cells or methods of producing modified cells that express one or more factors found in Table 4. The modified cells can be made by a method comprising expressing one or more factors from Table 4 in adipose-derived stem cells. These adipose cells can be used in food products, including, in combination with muscle cells to produce a mixture of cells for the food product.

TABLE 4
List of genes that have been ectopically expressed to induce
adipogenesis in various adipogenic precursor cells.

| Adipogenic Gene | Species | Cell Types |
|---|---|---|
| Peroxisome proliferator-activated | Human (SEQ ID NO: 75), | PSCs/MSCs, Myoblasts, |
| receptor gamma (PPARγ) | Mouse, (SEQ ID NO: 77) | Fibroblasts |
| | Chicken SEQ ID NO: 76), | |
| | Cow (SEQ ID NO: 73), | |
| | Goat (SEQ ID NO: 78), | |
| | Pig (SEQ ID NO: 79) | |
| CCAAT/enhancer-binding protein | Mouse (SEQ ID NO: 80), | Myoblasts, Fibroblasts, |
| alpha(C/EBPα) | Chicken (SEQ ID NO: 81), | Preadipocytes |
| | Goat (SEQ ID NO: 82) | |
| | Cow (SEQ ID NO: 74) | |
| Sterol regulatory element-binding | Mouse (SEQ ID NO: 83), | Fibroblasts |
| protein-1 (SREBP-1) | Chicken (SEQ ID NO: 84) | |
| Fatty Acid Binding Protein 4 (FABP4) | Cow (SEQ ID NO: 85) | MSCs |
| Zinc finger protein 423 (Zfp423) | Cow (SEQ ID NO: 86) | MSCs |
| Zinc finger and BTB domain containing | Cow (SEQ ID NO: 87) | DFAT cells |
| 16 (ZBTB16) | | |
| Perilipin 1 (PLIN1) | Cow (SEQ ID NO: 88) | Preadipocytes |
| Kruppel-like factor 13 (KLF13) | Pig (SEQ ID NO: 89) | MSCs |
| Phosphoenolpyruvate carboxykinase 1 | Buffalo (SEQ ID NO: 90) | Preadipocytes |
| (PCK1) | | |
| Early B-Cell Factor (Ebf) 1 | Mouse (SEQ ID NO: 91) | Fibroblasts |
| Runt-related transcription factor 1 | Mouse (SEQ ID NO: 92) | MSCs |
| (RUNX1) | | |
| Cyclin-Dependent Kinase 4 (CDK4) | Mouse (SEQ ID NO: 93) | Fibroblasts |
| Early B-Cell factor (Ebf) 2 | Mouse (SEQ ID NO: 98) | Fibroblasts |
| Early B-Cell factor (EBF) 3 | Mouse (SEQ ID NO: 99) | Fibroblasts |
| CCAAT/enhancer-binding protein beta | Mouse (SEQ ID NO: 94) | Myoblasts, Fibroblasts, |
| (C/EBPβ) | Chicken (SEQ ID NO: 95) | Preadipocytes |
| | Goat (SEQ ID NO: 96) | |
| | Bovine (SEQ ID NO: 97) | |

While the above work lists several relevant growth factors, a wide range of possible targets exist (along with their associated receptors), including: FGF (Fibroblast growth factor), TGF (transforming growth factor), IGF (insulin-like growth factor), PDGF (platelet-derived growth factor), CT1 (Cardiotropin), HGF (Hepatocyte growth factor), EGF (epidermal growth factor), PEDF (Pigment epithelium-derived factor), GH (growth hormone), IL-6 (interleukin 6), LIF (Leukemia inhibitory factor), TNFa (Tumor necrosis factor), VEGF (Vascular endothelial growth factor), additional options include the production of hormones or steroidal molecules and micro RNAs (miRNAs). In addition, other factors comprising the ectopic or expression of pro-proliferative metabolites are introduced.

In one embodiment, controlling signaling cascades without ectopic growth factor expression, for example, by using a constitutively active receptor for a growth factor. For instance, a mutation in the FGF receptor is known to lead to a “permanently on” status, which would lead to permanent signaling. This mutation is involved in Crouzon syndrome, and the associated mutation could induce FGF signal transduction in bovine muscle cells to achieve similar outcomes desired by the present invention. In one embodiment, Crispr/Cas9 can be used to provide the mutation into a muscle cell. In another embodiment, a vector comprising the mutant FGFr is introduced into the cell.

TABLE 2
List of growth factors and sequences for use in the methods and compositions disclosed herein.

| SEQ ID NO: | Protein name | Source Organism | Protein sequence | |
|---|---|---|---|---|
| 1 | Neuregulin 1 | Bos taurus | MEIYSPDMSE GAAERSSSPS TQLSADPSLD GLPAAEDMPE TQTEDGRSPG LVGLTLPCCV | 60 |
| | | | CLETERLRGC LNSEKICIVP ILACLVSLCL CIAGLKWVFV DKIFEYDSPT HLDPGGLGQD | 120 |
| | | | PIISLDPTAA SAAVWVSAKA YTSPVSRAQS ETEVRVTVQV DNALGSSEPS AVPTPKNRIF | 180 |
| | | | AFSFRPSTAP SLPSPTRSPD VRTPKAATQP PSTETNLQTA PKLSTSTSTA GTSHLVKCAE | 240 |
| | | | KEKTFCVNGG ECFMVKDLSN PSRYLCKCPN EFTGDRCQNY VMASFYKHLG IEFMEAEELY | 300 |
| | | | QKRVLTITGI CIALLVVGIM CVVAYCKTKK QRKKLHDRLR QSLRSERNTM MNVANGPHHP | 360 |
| | | | NPPPENVQLV NQYVSKNVIS SEHIVEREAE TSFSTSHYTS TAHHSTTVTQ TPSHSWSNGH | 420 |
| | | | TESIISESHS VIVMSSVENS RHSSPTGGPR GRLNGLGGPR ECNSFLRHAR ETPDSYRDSP | 480 |
| | | | HSERYVSAMT TPARMSPVDF HTPSSPKSPP SEMSPPVSST TVSMPSMAVS PFVEEERPLL | 540 |
| | | | LVTPPRLREK YDHHAQQFNS FHCNPAHESN SLPPSPLRIV EDEEYETTQE YEPAQEPVKK | 600 |
| | | | LTNSSRRAKR TKPNGHIAHR LEMDNNTGAD SSNSESETED ERVGEDTPFL AIQNPLAASL | 660 |
| | | | EAAPAFRLVD SRTNPTGRES PQEELQARLS GVIANQDPIA V | 701 |
| 2 | Neuregulin 1 | Oreochromis | MEEVSAGPAP PFGSVSPTGS TVDTRQVPEE KPEETEAAGE SGEEAIEGTA EGGGAGDDGE | 60 |
| | | niloticus | HVGAFGIVTL PEACCVCIEM EQINNCLHSE KICILPILAC LLSLALCTAG LKWVFVDKIF | 120 |
| | | | EYEPPSHLDP KPIGQDPIII PVEPTLGITV SFPHTTPSNN SLTTTFTLTP EHPEVSVKDK | 180 |
| | | | STQHHHQTIC KCLFPATIST STTAKTSSHV TRCSDSQRNY CVNGGECFTL EIIPGSTKCP | 240 |
| | | | VEFTGDRCQN YVMASFYKWL SFFSEAEELY QKRILTITGI CIALLVVGIM CVVAYCKTKK | 300 |
| | | | QRKKLRDRLR QSLRNKRKNT STGIDSNVAK STTGRPNSNL PLQDLQLIGV CCYFVSVVSH | 360 |
| | | | TAEKETETNF STSKYTLSVH DPTTLTNISS QSWSNDWSNS VQSDTESVSV MSLAENSQRA | 420 |
| | | | TQGARGRLNA TGGTSDLSAH SKKFVPVMTT LIQLTSESQN SLATPGSPPS EISAPISSLA | 480 |
| | | | ISVPSVTLSP SGEEERPLLH QLHKSSSRDV QKRTSSHYNH GHVADSLPSS PLFTMENADY | 540 |
| | | | QTIQDTRAAS CMFSTAPVKL INTNNNVSDC SNKSVTDHVV DYLRINGDSI LVSDTEEPRG | 600 |
| | | | EHIPFLSADR NTALLLRAID SSRTNPASPN DDLPVKSSSL INQQDSIAA | 649 |
| 3 | Insulin | Bos taurus | MALWTRLRPL LALLALWPPP PARAFVNQHL CGSHLVEALY LVCGERGFFY TPKARREVEG | 60 |
| | | | PQVGALELAG GPGAGGLEGP PQKRGIVEQC CASVCSLYQL ENYCN | 105 |
| 4 | Insulin | Oreochromis | MAALWLQAFS LLVLMMVSWP GSQAVGGPQH LCGSHLVDAL YLVCGDRGFF YNPRRDVDPL | 60 |
| | | niloticus | LGFLPPKAGG AVVQGGENEV TFKDQMEMMV KRGIVEECCH KPCTIFDLQN YCN | 113 |
| 5 | Serotransferrin | Bos taurus | MRPAVRALLA CAVLGLCLAD PERTVRWCTI STHEANKCAS FRENVLRILE SGPFVSCVKK | 60 |
| | | | TSHMDCIKAI SNNEADAVTL DGGLVYEAGL KPNNLKPVVA EFHGTKDNPQ THYYAVAVVK | 120 |
| | | | KDTDFKLNEL RGKKSCHTGL GRSAGWNIPM AKLYKELPDP QESIQRAAAN FFSASCVPCA | 180 |
| | | | DQSSFPKLCQ LCAGKGTDKC ACSNHEPYFG YSGAFKCLME GAGDVAFVKH STVEDNLPNP | 240 |
| | | | EDRKNYELLC GDNTRKSVDD YQECYLAMVP SHAVVARTVG GKEDVIWELL NHAQEHFGKD | 300 |
| | | | KPDNFQLFQS PHGKDLLFKD SADGFLKIPS KMDFELYLGY EYVTALQNLR ESKPPDSSKD | 360 |
| | | | ECMVKWCAIG HQERTKCDRW SGESGGAIEC ETAENTEECI AKIMKGEADA MSLDGGYLYI | 420 |
| | | | AGKCGLVPVL AENYKTEGES CKNTPEKGYL AVAVVKTSDA NINWNNLKDK KSCHTAVDRT | 480 |
| | | | AGWNIPMGLL YSKINNCKED EFFSAGCAPG SPRNSSLCAL CIGSEKGTGK ECVPNSNERY | 540 |
| | | | YGYTGAFRCL VEKGDVAFVK DQTVIQNTDG NNNEAWAKNL KKENFEVLCK DGTRKPVTDA | 600 |
| | | | ENCHLARGPN HAVVSRKDKA TCVEKILNKQ QDDFGKSVTD CTSNFCLFQS NSKDLLFRDD | 660 |
| | | | TKCLASIAKK TYDSYLGDDY VRAMTNLRQC STSKLLEACT FHKP | 704 |
| 6 | Serotransferrin | Oreochromis | VAMWRTVGTL LLILHTVFGQ STIKWCTISQ NEQRKCQAMS QAFSTASIRP SLSCVNAASV | 60 |
| | | niloticus | EDCGQKLQRK EADALSMSAK DIYNLGKTVS LKMAGSESQA GSVTTYYAVA VVKKENSGIN | 120 |
| | | | INNLAGKKSC HTGIGRTVGW NMPIGYLIDQ GYMSVMGCNI PQGVANFFSA SCIPGATQGE | 180 |
| | | | PQSLCQLCRG DESGQHKCEM SSNERYYSYE GAFRCLADGA GEVAFTKDTT VEENTDGRGP | 240 |
| | | | TWAKDLKSSD YELLCPDGTR APVTQWSRCN LVPVPSRGVV VRNDISPSVV FNMLMEGLEK | 300 |
| | | | SNFPMESSAG YGDGTVLESN LTTTFKAANS DEPKKWMGER YHNALKAMDC DPKDIPNALR | 360 |
| | | | WCVISSGEQQ KCADMSVAFQ SKGLTPNINC VYGDSVTHCM KKIKDNDADA ITLDGGYIYT | 420 |
| | | | AGKDYGLVPA TGESYTDDHD GSSYYAVAVV KKSSFDIRNL DDLRGRRSCH TGYGRTAGWN | 480 |
| | | | IPVAVLMERG LISPQQCQIP QAVGDFFEKC CVPGANQAGF PSNLCELCVG DESGQNKCEK | 540 |
| | | | GKDLYDGYDG AFRCVAKGEG DVAFVKHSTV LENTAGKTSL PPSDFQLLCQ SGGKAEATHY | 600 |
| | | | KYCNLGRVPS HAVMVRPDMN IHAIYGLLDQ AQTYFGSDTG TAFKMFDSQV YKGTDLIFKD | 660 |
| | | | STVRLVGVAD RKTYQEWLGQ IYLDSLVDLE CSSSNAVASS VWLLPTALFS FMLTNYWM | 718 |
| 7 | FGF1 | Bos taurus | MAEGETTTFT ALTEKFNLPL GNYKKPKLLY CSNGGYFLRI LPDGTVDGTK DRSDQHIMAE | 60 |
| | | | GETTTFTALT EKENLPLGNY KKPKLLYCSN GGYFLRILPD GTVDGTKDRS DQHIQLQLCA | 120 |
| | | | ESIGEVYIKS TETGQFLAMD TDGLLYGSQT PNEECLFLER LEENHYNTYI SKKHAEKHWF | 180 |
| | | | VGLKKNGRSK LGPRTHFGQK AILFLPLPVS SDMAEGETTT FTALTEKENL PLGNYKKPKL | 240 |
| | | | LYCSNGGYFL RILPDGTVDG TKDRSDQHIQ LQLCAESIGE VYIKSTETGQ FLAMDTDGLL | 300 |
| | | | YGSQTPNEEC LFLERLEENH YNTYISKKHA EKHWFVGLKK NGRSKLGPRT HFGQKAILFL | 360 |
| | | | PLPVSSDQLQ LCAESIGEVY IKSTETGQFL AMDTDGLLYG SQTPNEECLF LERLEENHYN | 420 |
| | | | TYISKKHAEK HWFVGLKKNG RSKLGPRTHF GQKAILFLPL PVSSD | 465 |
| 8 | FGF1 | Oreochromis | MRPIPSRLSC VFLHLFALFY YAQVTNQSPP NFTQHVSEQS KVTDRTSRRL IRIYQLYSRT | 60 |
| | | niloticus | SGKHVQVLPN KKINAMAEDG DVHAKLIVET DTFGSRVRIR GAETGLYICM NKRGKLIGKK | 120 |
| | | | NGQGRDCIFT EIVLENNYTA LKNVRYEGWY MAFTRQGRPR KGSRTRQHQR EVHEMKRLPK | 180 |
| | | | GHQPTHPSHH QPFDFIHYPF SQRTKRT | 207 |
| 9 | FGFR1 | Bos taurus | MWSRKCLLFW AVLVTATLCT AKPAPTLPEQ AQPWGAPVEV ESLLVHPGDL LQLRCRLRDD | 60 |
| | | | VQSINWLRDG VQLADSNRTR ITGEEVEVRG SVPADSGLYA CVTSSPSGSD TTYFSVNVSD | 120 |
| | | | ALPSSEDDDD DDDSSSEEKE TDNTKPNRMP VAPYWTSPEK MEKKLHAVPA AKTVKFKCPS | 180 |
| | | | SGTPNPTLRW LKNGKEFKPD HRIGGYKVRY ATWSIIMDSV VPSDKGNYTC IVENEYGSIN | 240 |
| | | | HTYQLDVVER SPHRPILQAG LPANKTVALG SNVEFMCKVY SDPQPHIQWL KHIEVNGSKI | 300 |
| | | | GPDNLPYVQI LKHSGINSSD AEVLTLENVT EAQSGEYVCK VSNYIGEANQ SAWLTVTRPV | 360 |
| | | | AKALEERPAV MTSPLYLEII IYCTGAFLIS CMVGSVIIYK MKSGTKKSDF HSQMAVHKLA | 420 |
| | | | KSIPLRRQVT VSADSSASMN SGVLLVRPSR LSSSGTPMLA GVSEYELPED PRWELPRDRL | 480 |
| | | | VLGKPLGEGC FGQVVLAEAI GLDKDRPNRV TKVAVKMLKS DATEKDLSDL ISEMEMMKMI | 540 |
| | | | GKHKNIINLL GACTQDGPLY VIVEYASKGN LREYLQARRP PGLEYCYNPS HHPEEQLSSK | 600 |
| | | | DLVSCAYQVA RGMEYLASKK CIHRDLAARN VLVTEDNVMK IADFGLARDI HHIDYYKKTT | 660 |
| | | | NGRLPVKWMA PEALFDRIYT HQSDVWSFGV LLWEIFTLGG SPYPGVPVEE LFKLLKEGHR | 720 |
| | | | MDKPSNCTNE LYMMMRDCWH AVPSQRPTFK QLVEDLDRIV ALTSNQEYLD LSMPLDQYSP | 780 |
| | | | SFPDTRSSTC SSGEDSVFSH EPLPEEPCLP RHPAQLANGG LKRRDYKDDD DK | 832 |
| 10 | FGFR1 | Oreochromis | MSQTPEWSSR RTKANSRSSV SRMLMRPSIL LFLALFAQVL RTQCRPANAD EVSVETQAEL | 60 |
| | | niloticus | YTLSIGDRLD LSCCAKDYLH AVNWTKDHVA VVDGEHTRIR NGQLEIESVE LTDSGLYTCT | 120 |
| | | | TFGNHSIFFN VTVHISASSE DDDDGEESSS EENKLLGSQK LMPMAPQWAH PEKMEKKLHA | 180 |
| | | | VPASKTVKFK CQASGNPTPT LKWYKNGKEF KRDHRIGGFK VRDHMWTIIM ESVVPSDKGN | 240 |
| | | | YTCVVENKYG SINHTYQLDV VERSPHRPIL QAGLPANRTV VVGSDVEFEC KVFSDPQPHI | 300 |
| | | | QWLKHIEVNG NRTGPDGLPY VRVLKHSGVN SSDAQVLTLY NVTEEESGEY ICKVSNYIGE | 360 |
| | | | VSQSAWLTVI RYEPTAPPHY SPASHTYLEV VIYCVGFFFI FAIMIAIAII VKIRTTSSKK | 420 |
| | | | SDENSQLAVH KLAKSIPLRR QVTVSVDSSS SIHSGVMLVR PSRLSSSGSP MLSGVSECEL | 480 |
| | | | PQDPRWELPR DRLVLGKPLG EGCFGQVVMG EAVGLDKEKP NRVTKVAVKM LKADATEKDL | 540 |
| | | | SDLISEMEMM KIIGKHKNII NLLGACTQDG PLYVIVEYAS KGNLREYLRA RRPPGMEYCS | 600 |
| | | | NPDQVPVENV SIKDLVSFAY QVARGMEYLA SKKCIHRDLA ARNVLVTDDS VMKIADFGLA | 660 |
| | | | RDIHHIDYYK KTTNGRLPVK WMAPEALFDR IYTHQSDVWS FGVLLWEIFT LGGSPYPGVP | 720 |
| | | | VEELFKLLKE GHRMDKPSTC THELYMMMRD CWHAVPSQRP TFKQLVEDLD RALAMTSNQE | 780 |
| | | | YLELSVPLDQ YSPSYPDTRS STCSSGEDSV FSHDAGAEEP CLPKFPPHSN GAAIKKR | 837 |
| 11 | FGFR2 | Bos taurus | MVSWGRFLCL VVVTMATLSL ARPSENLVDD TTVEPEEPPT KYQISQPEVY VAAPRESLEL | 60 |
| | | | RCLLRDAAMI SWTKDGVHLG PNNRTVLIGE YLQIKGATPR DSGLYACTAA RNVDSETVYF | 120 |
| | | | MVNVTDAISS GDDEDDADGS EDFVSENSNS KRAPYWTNTE KMEKRLHAVP AANTVKFRCP | 180 |
| | | | AGGNPTPTMR WLKNGKEFKQ EHRIGGYKVR NQHWSLIMES VVPSDKGNYT CVVENDYGSI | 240 |
| | | | NHTYHLDVVE RSPHRPILQA GLPANASTVV GGDVEFVCKV YSDAQPHIQW IKHVEKNGSK | 300 |
| | | | YGPDGLPYLK VLKAAGVNTT DKEIEVLYIR NVTFEDAGEY TCLAGNSIGI SFHSAWLTVL | 360 |
| | | | PAPVREKEIP ASPDYLEIAI YCIGVFFIAC MVVTVILCRM RNTTKKPDFS SQPAVHKLTK | 420 |
| | | | RIPLRRQVSA ESSSSMNSNT PLVRITTRLS STADTPMLAG VSEYELPEDP KWEFPRDKLT | 480 |
| | | | LGKPLGEGCF GQVVMAEAVG IDKEKPKEAV TVAVKMLKDD ATEKDLSDLV SEMEMMKMIG | 540 |
| | | | KHKNIINLLG ACTQDGPLYV IVEYASKGNL REYLRARRPP GMEYSYDINR VPEEQMAFKD | 600 |
| | | | LVSCTYQLAR GMEYLASQKC IHRDLAARNV LVTENNVMKI ADFGLARDIN NIDYYKKTTN | 660 |
| | | | GRLPVKWMAP EALFDRVYTH QSDVWSFGVL MWEIFTLGGS PYPGIPVEEL FKLLKEGHRM | 720 |
| | | | DKPANCTNEL YMMMRDCWHA VPSQRPTFKQ LVEDLDRILT LTTNEEYLDL SQPLEPYSPC | 780 |
| | | | YPDPR | 785 |
| 12 | TGFb1 | Bos taurus | MPPSGLRLLP LLLPLLWLLM LTPGRPVAGL STCKTIDMEL VKRKRIEAIR GQILSKLRLA | 60 |
| | | | SPPSQGDVPP GPLPEAILAL YNSTRDRVAG ESAETEPEPE ADYYAKEVTR VLMVEYGNKI | 120 |
| | | | YDKMKSSSHS IYMFENTSEL REAVPEPVLL SRADVRLLRL KLKVEQHVEL YQKYSNNSWR | 180 |
| | | | YLSNRLLAPS DSPEWLSFDV TGVVRQWLTR REEIEGFRLS AHCSCDSKDN TLQVDINGES | 240 |
| | | | SGRRGDLATI HGMNRPFLLL MATPLERAQH LHSSRHRRAL DTNYCFSSTE KNCCVRQLYI | 300 |
| | | | DFRKDLGWKW IHEPKGYHAN FCLGPCPYIW SLDTQYSKVL ALYNQHNPGA SAAPCCVPQA | 360 |
| | | | LEPLPIVYYV GRKPKVEQLS NMIVRSCKCS | 390 |
| 13 | TGFb1 | Oreochromis | MNLGKALLFF LLSNCVTVTF LLSTCTTVDI KHIKKKRVEA VRGQILSKLR LTSPPQTTGP | 60 |
| | | niloticus | SQVPFQVLAL YNSTKELIEE LGKDRQQSCG QDNTETEYYA KEIYKENMIN GPQENNDLPN | 120 |
| | | | CPRGITPKIF RFNVSIMEKN ATNLFRAEFR ALRIPNPSAR RNEQRIELYQ ILQKDDPKAK | 180 |
| | | | QRYIGGKNVL TKGTPEWVSF DVTETVREWL MNRRTNLGLE ISVHCPCHTF RPNGDIIENA | 240 |
| | | | NEVLEVKFKG MEAEYDEQSR VKSQKEQLYP HLILMMLPPH RLDAQSSSRR RKRALDTNYC | 300 |
| | | | FNNYEENCCV RQLYINFRED LGWRWIHQPE GYYANFCSGP CPYLRSADTT HSSLLSLYNT | 360 |
| | | | LNPEASASPC CVPQDLEPLT ILYYVGRSPK VEQLSNMVVK SCKCS | 405 |
| 14 | TGFR1 | Bos taurus | MEAAAATPRP RLFLLMLAAA ATLVPEATPL QCFCHLCTKD NFTCVTDGLC FVSVTETTDK | 60 |
| | | | VIHNSMCIAE IDLIPRDRPF VCAPSSKTGS ITTTYCCNQD HCNKIELPTV GKPSSGLGPV | 120 |
| | | | ELAAVIAGPV CFVCISLMLM VYICHNRTVI HHRVPNEEDP SLDRPFISEG TTLKDLIYDM | 180 |
| | | | TTSGSGSGLP LLVQRTIART IVLQESIGKG RFGEVWRGKW RGEEVAVKIF SSREERSWER | 240 |
| | | | EAEIYQTVML RHENILGFIA ADNKDNGTWT QLWLVSDYHE HGSLFDYLNR YTVTVEGMIK | 300 |
| | | | LALSTASGLA HLHMEIVGTQ GKPAIAHRDL KSKNILVKKN GTCCIADLGL AVRHDSATDT | 360 |
| | | | IDIAPNHRVG TKRYMAPEVL DDSINMKHFE SFKRADIYAM GLVFWEVARR CSIGGIHEDY | 420 |
| | | | QLPYYDLVPS DPSVEEMRKV VCEQKLRPNI PNRWQSCEAL RVMAKIMREC WYANGAARLT | 480 |
| | | | ALRIKKTLSQ LSQQEGIKM | 499 |
| 15 | TGFb3 | Bos taurus | MHLLAKPQSS GSREAAWFSS LLLHDCRGLL LPGLAAFLPG PRLKMHLQRA LVVLALLNFA | 60 |
| | | | TVSLSMSTCT TLDENHIKRK RVEAIRGQIL SKLRLTSPPD PSGLASVPIQ VLDLYNSTRE | 120 |
| | | | LLEEVHGERG DVCTQANTES EYYAKEIYKF DMIQGLEEHN DLTVCPKGIT SKIFRENVSS | 180 |
| | | | VEKNETNLFR AEFRVERMPN PASKRSEQRI ELFQILQPGE HIAKQRYIDG KNLPTRGTGE | 240 |
| | | | WLSFDVTDTV REWLLRRESN LGLEISIHCP CHTFQPNGDI LENIQELMEI KFKGVDSDDD | 300 |
| | | | PGRGDLGRLK KKKEHIPHLI LMMIPPNRLD SPGHSQRKKR ALDTNYCERN LEENCCVRPL | 360 |
| | | | YIDFRQDLGW KWVHEPKGYY ANFCSGPCPY LRSSDTTHST VLGLYNTLNP EASASPCCVP | 420 |
| | | | QDLEPLTILY YVGRTPKVEQ LSNMVVKSCK CS | 452 |
| 16 | TGFb3 | Oreochromis | MNLGKALLFF LLSNCVTVTF LLSTCTTVDI KHIKKKRVEA VRGQILSKLR LTSPPQTTGP | 60 |
| | | niloticus | SQVPFQVLAL YNSTKELIEE LGKDRQQSCG QDNTETEYYA KEIYKENMIN GPQENNDLPN | 120 |
| | | | CPRGITPKIF RENVSIMEKN ATNLFRAEFR ALRIPNPSAR RNEQRIELYQ ILQKDDPKAK | 180 |
| | | | QRYIGGKNVL TKGTPEWVSF DVTETVREWL MNRRTNLGLE ISVHCPCHTF RPNGDIIENA | 240 |
| | | | NEVLEVKFKG MEAEYDEQSR VKSQKEQLYP HLILMMLPPH RLDAQSSSRR RKRALDTNYC | 300 |
| | | | FNNYEENCCV RQLYINFRED LGWRWIHQPE GYYANFCSGP CPYLRSADTT HSSLLSLYNT | 360 |
| | | | LNPEASASPC CVPQDLEPLT ILYYVGRSPK VEQLSNMVVK SCKCS | 405 |
| 17 | TFR2 | Oreochromis | MKARRCSLTF RGVLSAFLIS VLLEPGAAGL DVFQLHELCK FCDVEPTSCN GTGICKTDCS | 60 |
| | | niloticus | ISSICEKPTE VCVSIWRRNE TNLSIETLCH NPAEPLYGIT LDDYNSTTCV MKEKNTTTGL | 120 |
| | | | AHFCSCKGAE CNEELIFSQT VDPTPDPLES NDSLMSVVLV SMLPLLVMVI AFFGMFYWCR | 180 |
| | | | IHRQRRPNRQ WESSENFKRK PIAGGLDCSD ACAIMTDDDK SDSSSTHANS LNHNTEPLPI | 240 |
| | | | ELDLLVGKGR FAQVYKAKLK QTTWNQFETV AVKIFPYEEY ASWKNEKDIF SNTDLRHENI | 300 |
| | | | LHELTAEERK VEKQYWLITA FHPRGNLQEY LTHHVISWQE LQVLSSSLAR GVAHLHSDRL | 360 |
| | | | PCGRPKVPIV HRDLKSSNIL VKNDGTCCVC DFGLGLCLDS SLSVDDLANS GQVGTARYMA | 420 |
| | | | PEVLEARLNL ENIESFKQTD IYSMALVLWE MTSRCEAIGE VKDYEPAYGS KVREHPCVES | 480 |
| | | | MKDNVLRDRG RPEIPDSWLR HQGMAVMCAT IKECWDHDPE ARLTAHCIAE RISELEDEMG | 540 |
| | | | KLSSRSSSAE KIVEELKIPI EVEIPEEEVK ITEIQNIIAV DCSISDKK | 588 |
| 18 | IGF-1 | Bos taurus | MWSRKCLLFW AVLVTATLCT AKPAPTLPEQ AQPWGAPVEV ESLLVHPGDL LQLRCRLRDD | 60 |
| | | | VQSINWLRDG VQLADSNRTR ITGEEVEVRG SVPADSGLYA CVTSSPSGSD TTYFSVNVSD | 120 |
| | | | ALPSSEDDDD DDDSSSEEKE TDNTKPNRMP VAPYWTSPEK MEKKLHAVPA AKTVKFKCPS | 180 |
| | | | SGTPNPTLRW LKNGKEFKPD HRIGGYKVRY ATWSIIMDSV VPSDKGNYTC IVENEYGSIN | 240 |
| | | | HTYQLDVVER SPHRPILQAG LPANKTVALG SNVEFMCKVY SDPQPHIQWL KHIEVNGSKI | 300 |
| | | | GPDNLPYVQI LKHSGINSSD AEVLTLENVT EAQSGEYVCK VSNYIGEANQ SAWLTVTRPV | 360 |
| | | | AKALEERPAV MTSPLYLEII IYCTGAFLIS CMVGSVIIYK MKSGTKKSDF HSQMAVHKLA | 420 |
| | | | KSIPLRRQVT VSADSSASMN SGVLLVRPSR LSSSGTPMLA GVSEYELPED PRWELPRDRL | 480 |
| | | | VLGKPLGEGC FGQVVLAEAI GLDKDRPNRV TKVAVKMLKS DATEKDLSDL ISEMEMMKMI | 540 |
| | | | GKHKNIINLL GACTQDGPLY VIVEYASKGN LREYLQARRP PGLEYCYNPS HHPEEQLSSK | 600 |
| | | | DLVSCAYQVA RGMEYLASKK CIHRDLAARN VLVTEDNVMK IADFGLARDI HHIDYYKKTT | 660 |
| | | | NGRLPVKWMA PEALFDRIYT HQSDVWSFGV LLWEIFTLGG SPYPGVPVEE LFKLLKEGHR | 720 |
| | | | MDKPSNCTNE LYMMMRDCWH AVPSQRPTFK QLVEDLDRIV ALTSNQEYLD LSMPLDQYSP | 780 |
| | | | SFPDTRSSTC SSGEDSVFSH EPLPEEPCLP RHPAQLANGG LKRRDYKDDD DK | 832 |
| 19 | IGF-1 | Oreochromis | MSSAFSFQWH LCDVFKSAMC CISCSHTLSL LLCVLTLTPT ATGAGPETLC GAELVDTLQF | 60 |
| | | niloticus | VCGERGFYEN KPTGYGPSAR RSRGIVDECC FQSCELQRLE MYCAPVKTPK ISRSVRSQRH | 120 |
| | | | TDMPRAPKVS SRANKGTERR TAPQPDKTKN KKEVHQKNSS RGSSGGRNYR M | 171 |
| 20 | IGF1R | Bos taurus | NAIFVPRPER KRREVMQIAN TTMSSRSRNT TVLDTYNITD PEELETEYPF FESRVDNKER | 60 |
| | | | TVISNLRPFT LYRIDIHSCN HEAEKLGCSA SNFVFARTMP AEGADDIPGP VTWEPRPENS | 120 |
| | | | IFLKWPEPEN PNGLILMYEI KYGSQVEDQR ECVSRQEYRK YGGAKLNRLN PGNYTARIQA | 180 |
| | | | TSLSGNGSWT DPVFFYVQAK TTYENFIHLM IALPIAVLLI VGGLVIMLYV FHRKRNSSRL | 240 |
| | | | GNGVLYASVN PEYFSAADVY VPDEWEVARE KITMSRELGQ GSFGMVYEGV AKGVVKDEPE | 300 |
| | | | TRVAIKTVNE AASMRERIEF LNEASVMKEF NCHHVVRLLG VVSQGQPTLV IMELMTRGDL | 360 |
| | | | KSYLRSLRPE MENNPVLAPP SLSKMIQMAG EIADGMAYLN ANKFVHRDLA ARNCMVAEDF | 420 |
| | | | TVKIGDFGMT RDIYETDYYR KGGKGLLPVR WMSPESLKDG VFTTHSDVWS FGVVLWEIAT | 480 |
| | | | LAEQPYQGLS NEQVLRFVME GGLLDKPDNC PDMLFELMRM CWQYNPKMRP SFLEIISSVK | 540 |
| | | | DEMEAGFREV SFYYSEENKP PEPEELDLEP ENMESVPLDP SASSASLPLP DRHSGHKAEN | 600 |
| | | | GPGPGVLVLR ASFDERQPYA HMNGGRKNER ALPLPQSSTC | 640 |
| 21 | IGFIR | Oreochromis | FGMVYEGIAK GVVKDEPETR VAIKTVNESA SMRERIEFLN EASVMREFNC HHVVRLLGVV | 60 |
| | | niloticus | SQGQPTLVIM ELMTRGDLKS YLRSLRPKEQ QWSSLSLPPL KKMLQMAGQI ADGMAYLNAN | 120 |
| | | | KFVHRDLAAR NCMVAEDFTV KIGDFGMT | 148 |
| 22 | PDGF | Bos taurus | MRTWACLLLL GCGYLANALA EEAEIPREVI ERLAHSQIHS IRDLQRLLEI DSVGAEEPLE | 60 |
| | | | TSLRAHGGHG AKHALEKRPV PIRRKRSIEE AIPAVCKTRT VIYEIPRSQV DPTSANFLIW | 120 |
| | | | PPCVEVKRCT GCCNTSSVKC QPSRVHHRNV KVAKVEYFRK KAKLKEVQVR LEEHLECTCT | 180 |
| | | | SASPSPDHRE EEAGRRRESG KKRKRKRLKP T | 211 |
| 23 | PDGF | Oreochromis | MRAAVYHILA GYLCVLRTVA QESPLPQELL ERLSRSEIRS ISDLQRLLEI DSVENEVSEE | 60 |
| | | niloticus | TKHSYHKDVS HSFPDLKRAH THTRHRRSAM VEEALPAVCK VRTAIYEIPR SQVDPTSANF | 120 |
| | | | IIWPPCVEVK RCTGCCNTSN MRCQAARKHY RTVKVAKVEY VRRRPKLREV QVRLEDHLEC | 180 |
| | | | MCTNKRHISP NSDTDNGIR | 199 |
| 24 | PDGFRa | Bos taurus | MGTSHWALLV LGCLFTGPSL ILCQLSLPSI LPNENERVVQ LNSSFSLRCF GESEVSWQYP | 60 |
| | | | MSEEEYPDVE IRNEENNSGL FVTVLEVVSA SAAHTGLYTC YYNHTQMDEN EIEGRHIYIY | 120 |
| | | | VPDPDVAFVP LGMTDSLVIV EDEDSVIIPC RTTDPETPVT LLSSEGIVPA SYDSRQGEKG | 180 |
| | | | TFSVGLYICE ATVRGKKFQT IPFNVYAYTA TSKLDLEMES PKTVYKAGES IVVTCTVENN | 240 |
| | | | EVVDFQWTYP GQMKGKGITR LEETKFPSIK LVYTLRVPEA TVKDSGDYEC AAHQATKEVK | 300 |
| | | | KMRNVTISVH EKGFIEIKPN FNLLEAVNLH EVKHFVVDVQ AYPPPKITWL KDNLTLIENL | 360 |
| | | | TEITTDIEKI QEISYRSKLK LIRAKEEDSG HYTIVVQNED DVKSYTFELL TQVPSSILDL | 420 |
| | | | VDDHHGSNGG QTVRCTAEGT PLPDIEWMVC KDIKKCNNET SWTVLANNVS NIITGVHPRD | 480 |
| | | | RSTVEGQVTF AKVEETIAVR CLAKNLLGVE SRELKLVAPT LRSELTVAAA VLVLLVIVII | 540 |
| | | | SLIVLVVIWK QKPRYEIRWR VIESISPDGH EYIYVDPMQL PYDSRWEFPR DGLVLGRILG | 600 |
| | | | SGAFGKVVEG TAYGLSRSQP VMKVAVKMLK PTARSSEKQA LMSELKIMTH LGPHLNIVNL | 660 |
| | | | LGACTKSGPI YIITEYCFYG DLVNYLHKNR DSFLSHHPEK PKKELDIFGL NPADESTRSY | 720 |
| | | | VILSFENNGD YMDMKQADTT QYVPMLERKE VSKYSDIQRA LYDRPASYKK KSMLDSEVKN | 780 |
| | | | LLSDDNSEGL TLLDLLSFTY QVARGMEFLA SKNCVHRDLA ARNVLLAQGK IVKICDFGLA | 840 |
| | | | RDIMHDSNYV SKGSTFLPVK WMAPESIFDN LYTTLSDVWS YGILLWEIFS LGGTPYPGMM | 900 |
| | | | VDSTFYNKIK SGYRMAKPDH ATSEVYEIMV KCWNSEPEKR PSFYHLSEIV ENLLPGQYKK | 960 |
| | | | SYEKIHLDFL KSDHPAVARM RVDSDNAYIG VTYKNEEDKL KDWEGGLDEQ RLSADSGYII | 1020 |
| | | | PLPDIDPVPE EEDLGKRNRH RIFLSLPHSV DYIKIFKQKH IASEKAMASY SSTLAWKIPW | 1080 |
| | | | MEEPGRLQSM GLLRVRHD | 1098 |
| 25 | PDGFRa | Oreochromis | KYSLFMTHCM CLLSAPTAGM IPSSSVPLLL PKLDEMVVTL HTPFTLTCHG QENLGWETPI | 60 |
| | | niloticus | DVSEQTQEDP NGLFISTITV DSATASHTGY YTCFYSRTFA EDTAEFSRIY IYVPDPDVPF | 120 |
| | | | LESLIPFGYH VLSGYDEMEI QCRVSDPNAN VTLINSDTQQ PVPSIYDSKR GALGIFTAGT | 180 |
| | | | YVCKAVINGH EFYSEEYIVH GSTDKDTNEL DVELTAKKTA LLVGDTITVT CVARGSEILE | 240 |
| | | | DHWKYPGKLA NRGTKTVREN KRTQEIFYTL TIPQASTKDS GIYSCSITDI LSNTSEFLSV | 300 |
| | | | KPEFREYESA ALDEVREFKA VVNSFPSVHV SWFKDGYPLS DVTAEISTSL QQTSEISYMS | 360 |
| | | | VLTLIRAKEE DSGNYTMRVE NRNQSRDISL ILEVKVPAVI VDLMDVHHGS AQGQSVVCIT | 420 |
| | | | RGQPTPMVEW FVCKNIKHCA NDSSSWVPIP ANSTEITLDS HIDEDNNLES QVMFGHLEST | 480 |
| | | | MAVRCLARNE MAVVSREVKL VSNGPHPELT VAAAVLVLLV IVIISLIVLV VIWKQKPRYE | 540 |
| | | | IRWRVIESVS PDGHEYIYVD PMQLPYDSRW EFPRDRLVLG RILGSGAFGK VVEGTAYGLS | 600 |
| | | | RSQPVMKVAV KMLKPTARSS EKQALMSELK IMTHLGPHLN IVNLLGACTK SGPIYIITEY | 660 |
| | | | CFYGDLVNYL HKNRENFLSL NPEKNKKELD IFGINPADES SRSYVILSFE SKGDYMDMKQ | 720 |
| | | | ADSTQYVPML EMSNASKYSD IQRSNYDHPP SQKGSSEMEI MLSDDVNEGL TTTDLLSFTY | 780 |
| | | | QVAKGMEFLA SKNCVHRDLA ARNVLLSQGK IVKICDFGLA RDIMHDNNYV SKGSTFLPVK | 840 |
| | | | WMAPESIFDN LYTTLSDVWS YGILLWEIFS LGGTPYPGMV VDSSFYNKIK SGYRMSKPEH | 900 |
| | | | APHDVYEMMM KCWNSEPEKR PSFLGLSETV ASLLPSSYKR QYERVNHEFL KSDHPAVTRV | 960 |
| | | | CMENEDYIGI AYKNQGKLKD RESGEDEQRL SSDSGYIIPL PDLDPISDEE YGKRNRHSSQ | 1020 |
| | | | TSEESAIETG SSSSTFAKRE GETLEDITLL DEMCLDCSDL VEDSFL | 1066 |
| 26 | PDGFRb | Bos taurus | MKDTMWLPSA MQAVVLKGRV LLLPLLFLLG PQASWGLAII PPGPELVLNL SSTFVLTCSG | 60 |
| | | | PAPVVWERMS QKPPQEMTET QDGTFSSVLT LVNVTGLDTG EYFCGYKRSH GLEASERKRL | 120 |
| | | | YIFVPDPSVG FLPVDPEELF IFLTEITETT IPCRVTDPRL VVMLHEKKVD VPLPIAYDPQ | 180 |
| | | | RGFSGTFEDK TYICKTTIGD REVDSDAYHV YSLQVSSINV SVNAVQTVVR QGENITIMCI | 240 |
| | | | VTGNEVVNFE WTYPRMESGR LVEPVTDFLF EMPHIRSILH IPSAELGDSG TYICNVSESV | 300 |
| | | | SDHRDEKAIN VTVVENGYVR LLGELDPVQF AELHRSRTLQ VVFEAYPPPT VMWFKDNRTL | 360 |
| | | | GDSSAGEIVL STRNVSETRY VSELTLVRVK VAEAGYYTMR AFHEDAEAQL SFQLQVNVPV | 420 |
| | | | RVLELSESHP ANGEQTVRCR GRGMPQPHLT WSTCSDLKRC PRELPPTPLG NSSEEESQLE | 480 |
| | | | TNVTYWAEEQ EFEVVSTLRL RHVDQPLSVR CTLHNLLGYD VQEVTVVPHS LPFKVVVISA | 540 |
| | | | ILALVVLTII SLIILIMLWQ KKPRYEIRWK VIESVSSDGH EYIYVDPMQL PYDSTWELPR | 600 |
| | | | DQLVLGRTLG SGAFGQVVEA TAHGLSHSQA TMKVAVKMLK STARSSEKQA LMSELKIMSH | 660 |
| | | | LGPHLNVVNL LGACTKGGPI YIITEYCRYG DLVDYLHRNK HTFLQRCSDK RRPPSAELYS | 720 |
| | | | NALPVGLPLP SHVSLPGESD GGYMDMSKDE SVDYVPMLDM KGDVKYADIE SSNYMAPYDN | 780 |
| | | | YVPSAPERTC RVTLINESPV LSYTDLVGFS YQVANGMEFL ASKNCVHRDL AARNVLICEG | 840 |
| | | | KLVKICDFGL ARDIMRDSNY ISKGSTFLPL KWMAPESIFN SLYTTLSDVW SFGILLWEIF | 900 |
| | | | TLGGTPYPEL PMNEQFYNAI KRGYRMAQPA HASDEIYEIM QKCWEEKFEI RPPFSQLVLL | 960 |
| | | | LERLLGEGYK KKYQQVDEEF LRSDHPAILR SQARLPGENG LRSPLDSSSV LYTAVQPNEG | 1020 |
| | | | DNDYIIPLPD PKPEVADEGP LEGSPSLASS TLNEVNTSST ISCDSPLEPQ EEPEPEPQPA | 1080 |
| | | | SQVEPEPKWP PDSSCPGPRA EAEDSEL | 1107 |
| 27 | CT1 | Bos taurus | GPPPWPLKHP ALSSPRQEEG PAHSCPAQLR PLLPSPSPPP SNKLSPKSSR GRRSEPETSP | 60 |
| | | | PIGPQGCLQG PDTPSLRSLS TLQTTCCPPQ VPFATRPSEQ REDPQADSSA SPLPHLEAKI | 120 |
| | | | QQTHSLARLL TKYAEQLLQE YVQHQGDPFG LPGFSPPRLP VADLSDPAPG HAGLPVPERL | 180 |
| | | | RLDAAALAAL PPLLDVVRRL QGELNPRALR LLRRLEDAAR QVRALGAAVE AVLAALGAES | 240 |
| | | | RGPRPEPAAA AVAASTASAG VFPAKVLGER VCGLYLEWVS RTEADLGQLA PGGPA | 295 |
| 28 | LIF1Ra | Bos taurus | MMDISLCVKR PSWLVDRKRM RMASNSQWLL LTAVLLYLTN QVNSQKKSTP HDLKCVINNL | 60 |
| | | | KVWDCSWRAL FGAGRAASYE VCIEKRSRTC YLLEKNTTIP PLLPGHYEIT VHPLYEFGSS | 120 |
| | | | KSKFILNEKN ISLIPETPEI LNLSADFSTS TLHLKWNDKG SVFPHYSNVI WEIKVLHKKN | 180 |
| | | | MEIVKLVTYS TTLNGRDTIH HWNWTSDMPL ECAIHYVGIR CYIDDPQFSG HKEWSDWSLL | 240 |
| | | | KNISLPPDSQ TKVFPQDKVI LAGSDITVCC VTQEKVLSAQ IGSTHCPLIH LDGENVAIRI | 300 |
| | | | RNISASASSG TNVVESVEDN IFGTVVFAGY PPDIPQKLNC ETYDLKEIIC TWNPGRPTSL | 360 |
| | | | EGPRSTSYTL FESFSGKYVR FKGDEVSANE NYQLFLQILS SQEIHNFTLN AQNLLGQTES | 420 |
| | | | TLLVNITEKV HPRIPTSLKA KDINSTAVIL SWHLPGNFTK VKLLCQIEIN KTNSEHELRN | 480 |
| | | | VTMRGVESSN YLTTVDKLNP YTIYTFRIRC STDPFWKWSK WSNEKQCLTT EAIPSKGPDI | 540 |
| | | | WREWSSDGRN LIIYWKPLPI NEAYGKILSY NVSYSSDEET KSLSEIPDTQ HRAELQLDKN | 600 |
| | | | DYIISVVAKN SAGSSPPSKI ASMEIPNDDL KIEQALGMGN RILLTWNYDP NMTCDYVIKW | 660 |
| | | | CNSSQSGPCL MDWKKVPPNS TETVIESDQF RPGVRYNFSV YGCRNQGYQL LRSVIGYVEE | 720 |
| | | | LAPAVAPNFT VEDTSADSIL VKWEEIPVEE LRGFLRGYLF YFEKGERDTS KIMGLEPGRF | 780 |
| | | | DTKVKNITDI SQKTLRIADL QGKTSYHLVL RAYTGGGMGP ERSMFVVTKE NSEGLIIAIL | 840 |
| | | | IPVAVAVIIG VVTSILCYRK REWIKETFYP DIPNPENCKA LQFQKSVCEG NSALKTLEMN | 900 |
| | | | PCTPNNVEVL ETRSVALKIE DTEIISPVAE RPEDRSDAEP ENHVVVSYCP PVIEEELANP | 960 |
| | | | GAEGGGASQV IYIDVQSMYQ PQAKPEGELD SDPVGGAGYK PQMHLPVTST VDDLDADDDL | 1020 |
| | | | DKTAGYRPQA NVNTWNLVSP DSPRSTDSNS EIVSFGSPCS INSRQFLIPP KDEDSPKSSG | 1080 |
| | | | GGWSFTNFFQ NKPND | 1095 |
| 29 | HGF | Bos taurus | MWVTRLLPVL LLQHVLLHLL LLPIAIPYAE GQKKRRNTLH EFKRSAKTTL IKEDPLLKIK | 60 |
| | | | TKKMNTADQC ANRCIRNKGL PFTCKAFVED KARKRCLWFP FNSMSSGVKK EFGHEFDLYE | 120 |
| | | | NKDYIRNCII GKGGSYKGTV SITKSGIKCQ PWNSMIPHEH SFLPSSYRGK DLQENYCRNP | 180 |
| | | | RGEEGGPWCF TSNPEVRYEV CDIPQCSEVE CMTCNGESYR GPMDHTETGK ICQRWDHQTP | 240 |
| | | | HRHKFLPERY PDKGFDDNYC RNPDGKPRPW CYTLDPDTPW EYCAIKMCAH STMNDTDLPM | 300 |
| | | | QTTECIQGQG EGYRGTINTI WNGIPCQRWD SQYPHQHDIT PENFKCKDLR ENYCRNPDGA | 360 |
| | | | ESPWCFTTDP NIRVGYCSQI PKCDVSSGQD CYRGNGKNYM GSLSKTRSGL TCSMWDKNME | 420 |
| | | | DLHRHIFWEP DATKLNKNYC RNPDDDAHGP WCYTGNPLIP WDYCPISRCE GDTTPTIVNL | 480 |
| | | | DHPVISCAKT KQLRVVNGIP TRTNVGWMVS LKYRNKHICG GSLIKESWIL TARQCFPSRN | 540 |
| | | | KDLKDYEAWL GIHDVHGRGD EKRKQVLNVT QLVYGPEGSD LVLLKLARPA ILDDFVSTID | 600 |
| | | | LPNYGCTIPE KTTCSVYGWG YTGLINSDGL LRVAHLYIMG NEKCSQYHQG KVTLNESEIC | 660 |
| | | | AGAENIVSGP CEGDYGGPLV CEQHKMRMVL GVIVPGRGCA IPNRPGIFVR VAYYAKWIHK | 720 |
| | | | IILTYKAPQS | 730 |
| 30 | HGF | Oreochromis | MWIYKLVFGF VFVVSCSEGR RNALQDYQKT DSIQLMVHSD SSHLTKSRKL SLSKCAKACS | 60 |
| | | niloticus | RNKRLPFTCR AFVYDHKNRK CQWLSFDRNS PGVQSQQNMN YQLYEKKDYV RECIVGTGQS | 120 |
| | | | YRGRRSVTVS GILCQAWASP VPHEHKFMSK RFRKTDLREN YCRNPDNSAV GPWCFTTDPR | 180 |
| | | | PHFRHEECGI PQCSEVECIN CIGEDYRGPM DHTESGKECQ RWDLDDPHKH LYHPKRYPDK | 240 |
| | | | GLDDNYCRNP DGRQRPWCFT TDPNTPWEYC NITVCETPPK SNVVETTECY QGRGEGYRGT | 300 |
| | | | VDMTPTGLTC QRWDSQYPHN HTFLPEAYPC KDLRENFCRN PDGQEFPWCF TTDPRVRTMS | 360 |
| | | | CINIPQCGTQ NRPVSDCYER FGENYQGQQS RTRSNLPCAP WRDHSNSGER GMPTAGLEGN | 420 |
| | | | YCRNPDKDKH GPWCYTNNSA IPWDYCNVKP CNASQNTIQL GEDASVSCFV HKTTRIVGGS | 480 |
| | | | PVSISEGSWM VSIQKGSMHW CGGSLIRDEW VLTDRECESS CVPNLSEYRV WLGVSDIREG | 540 |
| | | | APDWSKRQEV SIAHVICGPE GSSLALLRLS KPALPADNVH TIQLPVAGCT IPEGTICKMY | 600 |
| | | | GWGETKGTGY EDMLKAVELP IVRNNRCREM HRGNLHITTN KICAGGKRNE GVCERDYGGP | 660 |
| | | | LVCQDGYIRV IVGVSVHGRG CARANQPSIF INVPFYTQWI YKVFKYYPNT V | 711 |
| 31 | HGFR | Bos taurus | MKAPAVLAPG ILVLLFTFVQ KSNGECKEAL VKSRMNVNMQ YQLPNFTAET SIQNVVLHKH | 60 |
| | | | HIYLGAINYI YVINDKDLQK VAEYKTGPVL EHPDCFPCQD CSHKANLSGG VWKDNINMAL | 120 |
| | | | LVDTYYDDQL ISCGSVHRGT CQRHVLPPNN TADIESEVHC MYSPQADEET NQCPDCVVSA | 180 |
| | | | LGTKVLLSEK DRFINFFVGN TINSSYLPDY ILHSISVRRL KETQDGFKFL TDQSYIDVLP | 240 |
| | | | ELRDSYPIKY VHAFESNHFI YFLTVQRETL DAQTFHTRII RFCSADSGLH SYMEMPLECI | 300 |
| | | | LTEKRRKRST KQEVENILQA AYVSKPGAQL ARQIGASLND DILYGVFAQS KPDSSEPMNR | 360 |
| | | | SAVCAFPVKY VNEFFNKIVN KNNVRCLQHF YGPNHEHCEN RTLLRNSSGC EVRNDEYRTE | 420 |
| | | | FTTALPRVDL FTGQFNQVLL TSISTFIKGD LTIANLGTSE GRFMQVVVSR SGSLTPHVNF | 480 |
| | | | HLDSHPVSPE VIVEHPLNQN GYTLVVTGKK ITKIPLNGLG CEHFQSCSQC LSAPSFVQCG | 540 |
| | | | WCHDKCVRLE ECSSGTWTQE TCLPTIYKVF PTSAPLEGGT TLTVCGWDFG FKRNNKFDLK | 600 |
| | | | KTRVLLGNES CTLTLTESTT NMLKCTVGPA MNEHFNMSIV ISNSRGSVEY SAFSYVDPII | 660 |
| | | | TSISPNYGPK TGGTLLTLTG KHLNSGNSRH ISIGGKTCTL KSVSHSILEC YTPAQSAPTE | 720 |
| | | | FSVKLKIDLA NREVNSFIYR EDPIVYEIHP TKSFISGGST ITGVGKNLNS VSVLRMVINV | 780 |
| | | | HEAGRNFTVA CQHRSNSEII CCTTPSIEQL NLQLPLKTKA FFMLDGIHSK YFDLIYVHNP | 840 |
| | | | VFKPFEKPVM ISVGNENVLE IKGNDIDPEA VKGEVLKVGN KSCENIHSHS EAVLCTVPSD | 900 |
| | | | LLKLNSELNI EWKQAISSTV LGKVIVQPDQ NFTGLIVGVV SISIILLLLL GLFLWLKKRK | 960 |
| | | | QIKDLGSELV RYDARVHTPH LDRLVSARSV SPTTEMVSNE SVDYRATFPE DQFPNASQNG | 1020 |
| | | | SCRQVQYPLT DLSPILTSGD SDISSPLLQN TIHIDLSALN PELVQAVQHV VIGPSSLIVH | 1080 |
| | | | FNEVIGRGHF GCVYHGTLLD NDDKKIHCAV KSLNRITDIG EVSQFLTEGI IMKDESHPNV | 1140 |
| | | | LSLLGICLRS EGSPLVVLPY MKHGDLRNFI RNETHNPTVK DLIGFGLQVA KGMEYLASKK | 1200 |
| | | | FVHRDLAARN CMLDEKFTVK VADFGLARDV YDKEYYSVHN KTGAKLPVKW MALESLQTQK | 1260 |
| | | | FTTKSDVWSF GVLLWELMTR GAPPYPDVNT FDITVYLLQG RRLLQPEYCP DPLYEVMLKC | 1320 |
| | | | WHPKAELRPS FSELVSRISV IFSTFIGEHY VHVNATYVNV KCVAPYPSLL SSQDNVSGED | 1380 |
| | | | DDDT | 1384 |
| 32 | HGFR | Oreochromis | MNLCLLHAIM LLWAIRSSVQ GQCDRSSEDT KLNLSVTYEL PSFTAEFPIQ NMVILDGVIY | 60 |
| | | niloticus | VGAVNRIYAL APNLRKLSEY HTGPLRANET CGQKLNGTSS SGRVDNHNIA LVAENIYDKG | 120 |
| | | | LYSCGSADNG VCRRHVLDDG VSAKSVDEEV YCFADKKKLE TGQPDDSDVV VSPLGSQVLN | 180 |
| | | | VESNVIQFFV GNSEIPGRRN ISSSAKHPHT LSLRKMKTSQ NGFTFFSNRS YMDLIPSLRG | 240 |
| | | | NYYLRYVYSF QSGPFTYFLT VQQVSKDSQA YHSRIVRMCS ADSVIRRYVE MPLECISTDK | 300 |
| | | | RRRRRRSGTG VKVFNILQAA YVTKAGIDPE IQKHLKVEED DDVLFAAFAK GKPNSKDPTP | 360 |
| | | | NSAVCVIALK QINSMFKTYM EKCNTIEPYH FTGLDKKSCY NVTSSDDCDP HEGIHEGKES | 420 |
| | | | TYRLQVTQFF QRLEYWHKDL TNTLVTSITV VPVHGRAVVY LGTADGRQIQ VLLSRFASPH | 480 |
| | | | VNIHLDSRPV TTSVALLDAD RSDGAILMAT GNKITKVPLI GPGCGQLTTC ISCLLSSRVT | 540 |
| | | | ECGWCDGRCT RASECTSSIW TQDYCTPEIT KVSPASGPIR GSTVVTICGK NFGEDKTENE | 600 |
| | | | KASMVTVEVA GASCKLSRQE NSNRWTEMHC SPVFNGNFTP SGDVVKVTSG QNVTMFKGEN | 660 |
| | | | FVDPVIRDIF PTFGPKSGNT MLTITGANLD TGSKREVTIG KGICNVQSWS SKKVTCKTPP | 720 |
| | | | YAALSSQTVK LTVDSVERRA PMQFTYNQDP IINNIQPSRS FVSGGCTVSA HGLYLKSGLQ | 780 |
| | | | PMMLVSIGEN TETFHVSCVY GENQTSIQCT TPSLVKLGMQ PPVITKVSFV LDGYSTKQMD | 840 |
| | | | MIYVEDPLFQ DPKLTSKDNK SIVELKGDRM DREAMNCQVL TVSNHSCESL TLVGNTLECI | 900 |
| | | | VPTELQVATA KELQVEWRQA DSIRHLGKVT LAQEQDYTGL IVGCVCVSLL LLLLGSLLMW | 960 |
| | | | KKNKHIDDQG TDRPETCRAP PPIYGGNGEL LSPRLGALGG GIGMGLGMGM GIDGDLVSPL | 1020 |
| | | | LMAPVHIDPS CLHPDLLTEV QHVVIARERL LLHLNQVIGR GHFGCVFHGT LLEPDGQKLH | 1080 |
| | | | CAVKSLNRIT DLEEVSQFLK EGIIMKDESH PNVLSLLGIC LPPEGSPLVV LPYMKHGDLR | 1140 |
| | | | NFIRDEGHNP TVKDLMGFGL QVARGMEYLA SKKFVHRDLA ARNCMLDESY TVKVADFGLA | 1200 |
| | | | RDVYDKEYYS VHNKSGVKLP VKWMALESLQ THKFTTKSDV WSFGVLLWEL MTRGAPPYSD | 1260 |
| | | | VNSFDITVFL LQGRRLLQPE FCPDALYTVM IECWHPKPER RPSFSELVSR ISAIFSSFSG | 1320 |
| | | | EHYVLLNTTY VNIDKMSPYP SLLSSTASSS SSSSEPSTST SLPSRSPLFC QVDRDCCT | 1378 |
| 33 | EGF | Bos taurus | IDECRRGVHS CGENATCTNM EGNHTCTCAG DLSEPGQICP DSTLLSHLGK NGHNFLKKCF | 60 |
| | | | PEYTPNFEGY CLNGRVCIYF GIANLFSCHC PIGYPGKRGE YIDFDGWDPH SAGRGHQWNT | 120 |
| | | | SPVAVRALVL AFLLLLGLCR AH | 142 |
| 34 | EGFR | Bos taurus | SRALEEKKVC QGTSNKLTQL GTFEDHELSL QRMENNCEVV LGNLEITYMQ SSYNLSFFKT | 60 |
| | | | IQEVAGYALI ALNTVEKIPL ENLQIIRGNV LYENTHALAV LSNYGANKTG LKELPL | 116 |
| 35 | EGFR | Oreochromis | MTPSHRELQN NTMAARFLKW ISLTSLLWLS FCGLAENKVC QGVTNRLNLL GTKEDHYRNM | 60 |
| | | niloticus | VKTYSNCTVV LENLEITYME DHRDLSFLRS IKEVGGYVLI ALNTVSRIPL ENLRIIRGHS | 120 |
| | | | LYDGSFALSV ISNYNKSTNK GTDQILLTSL TEILKGGVKI WTNQLCNVET IQWSDIVNVG | 180 |
| | | | NISIDPTPSK NKNCGKCDSS CENGSCWSPG PQNCQTLTKL NCAQQCSRRC KGPSPSDCCN | 240 |
| | | | EHCAAGCTGP RAEDCLACRD FQDNGVCKDS CPGLHRYDPN LHQLVPNPHG KYSFGATCVK | 300 |
| | | | SCPRNYIISD YGACVRTCNG NTYEVEVGGI RKCAKCDGLC PKVCNGLGTG DLVHTLSINA | 360 |
| | | | TNIGSFENCT KINGNIDIIH TSIYGDKFTK TPKMDPEQLN VFKTVKEITG YLWIQTWPDT | 420 |
| | | | MSSLSPFENL EIVRGRTKRG SRSLVVSGLH ITHLGLRSLR EISDGDVFVS KNSELCYTDA | 480 |
| | | | KHWQKLFKSP HQTVNIEGNA NAATCALRND TCDKKCTADG CWGPGPSMCI SCRDYKRDGS | 540 |
| | | | CVDSCNILEG EPRETAVNKT CIKCHPECQP MNGTATCSAP GSANCTKCAN FKDGLFCVSR | 600 |
| | | | CPQGVLGEDD ALVWKYADEQ KVCQLCHKNC TQGCIGPGLE GCHSKGPPGL SMVAAGVVGG | 660 |
| | | | LLAALIAGLS VEVLLRRRHI KRKRTMRRLL QERELVEPLT PSGEAPNQAL LRILKEPEFK | 720 |
| | | | KIKVLGSGAF GTVYKGLWVP EGEDVKIPVA IKVLREATSP KANKEILDEA YVMASVEHPH | 780 |
| | | | VCRLLGICLT STVQLITQLM PYGCLLDYVK ENKDNIGSQY LLNWCVQIAK GMNYLEERHL | 840 |
| | | | VHRDLAARNV LVKTPQHVKI TDFGLAKLLK ADEKEYHADG GKVPIKWMAL ESILNRTYTH | 900 |
| | | | QSDVWSYGVT VWELMTFGTK PYDGIPASEI AGILEKGERL PQPPICTIDV YMIMVKCWMI | 960 |
| | | | DADSRPRFRE LIAEFTKMAR DPPRYLVIQG DDRMHLPSPS DTKFYRSLIS GEDMEDAVDA | 1020 |
| | | | DEYLVPQRGF FSSPSTSRTP LIHSTSLNSS IGTCHSRTGL LNGFPSRDGS MALRYIPDPT | 1080 |
| | | | DKFLDEAFQP APGYINQTPI SDVVNPIYQH PGPPRTLLPT ISSDHTETEY LNYFKNGAAG | 1140 |
| | | | PEYLNEILSP AVLPLTSNGT VHSIEKYLPQ QSIDNPDYQQ DFTPTFKTHT NGHIPAAENA | 1200 |
| | | | EYLGPD | 1206 |
| 36 | PEDF | Bos taurus | MQALVLLLWT GALLGFGRCQ NAGQEAGSLT PESTGAPVEE EDPFFKVPVN KLAAAVSNFG | 60 |
| | | | YDLYRVRSGE SPTANVLLSP LSVATALSAL SLGAEQRTES NIHRALYYDL ISNPDIHGTY | 120 |
| | | | KDLLASVTAP QKNLKSASRI IFERKLRIKA SFIPPLEKSY GTRPRILTGN SRVDLQEINN | 180 |
| | | | WVQAQMKGKV ARSTREMPSE ISIFLLGVAY FKGQWVTKED SRKTSLEDFY LDEERTVKVP | 240 |
| | | | MMSDPQAVLR YGLDSDLNCK IAQLPLTGST SIIFFLPQKV TQNLTLIEES LTSEFIHDID | 300 |
| | | | RELKTVQAVL TIPKLKLSYE GELTKSVQEL KLQSLFDAPD FSKITGKPIK LTQVEHRVGF | 360 |
| | | | EWNEDGAGTN SSPGVQPARL TFPLDYHLNQ PFIFVLRDTD TGALLFIGKI LDPRGT | 416 |
| 37 | GH | Bos taurus | MMAAGPRTSL LLAFALLCLP WTQVVGAFPA MSLSGLFANA VLRAQHLHQL AADTFKEFER | 60 |
| | | | TYIPEGQRYS IQNTQVAFCF SETIPAPTGK NEAQQKSDLE LLRISLLLIQ SWLGPLQFLS | 120 |
| | | | RVFTNSLVFG TSDRVYEKLK DLEEGILALM RELEDGTPRA GQILKQTYDK FDTNMRSDDA | 180 |
| | | | LLKNYGLLSC FRKDLHKTET YLRVMKCRRF GEASCAF | 217 |
| 38 | GH | Oreochromis | MNSVVLLLSV VCLGVSSQQI TDSQRLFSIA VNRVTHLHLL AQRLFSDFES SLQTEEQRQL | 60 |
| | | niloticus | NKIFLQDFCN SDYIISPIDK HETQRSSVLK LLSISYGLVE SWEFPSRSLS GGSSLRNQIS | 120 |
| | | | PRLSELKTGI LLLIRANQDE AENYPDTDTL QHAPYGNYYQ SLGGNESLRQ TYELLACFKK | 180 |
| | | | DMHKVETYLT VAKCRLSPEA NCTL | 204 |
| 39 | GHR | Bos taurus | MDLWQLLLTL AVAGSSDAFS GSEATPAFLV RASQSLQILY PVLETNSSGN PKFTKCRSPE | 60 |
| | | | LETFSCHWTD GANHSLQSPG SVQMFYIRRD IQEWKECPDY VSAGENSCYF NSSYTSVWTP | 120 |
| | | | YCIKLTSNGG IVDHKCFSVE DIVQPDPPVG LNWTLLNISL TEIHADILVK WEPPPNTDVK | 180 |
| | | | MGWIILEYEL HYKELNETQW KMMDPLMVTS VPMYSLRLDK EYEVRVRTRQ RNTEKYGKES | 240 |
| | | | EVLLITFPQM NPSACEEDFQ FPWFLIIIFG ILGLAVTLYL LIFSKQQRIK MLILPPVPVP | 300 |
| | | | KIKGIDPDLL KEGKLEEVNT ILAIHDNYKH EFYNDDSWVE FIELDIDDPD EKTEGSDTDR | 360 |
| | | | LLSNDHEKSL NIFGAKDDDS GRTSCYEPDI LEADFHVSDM CDGTSEVAQP QRLKGEADIS | 420 |
| | | | CLDQKNQNNS PSNDAAPASQ QPSVILVEEN KPRPLLIGGT ESTHQAVHTQ LSNPSSLANI | 480 |
| | | | DFYAQVSDIT PAGNVVLSPG QKNKTGNPQC DTHPEVVTPC QANFIVDNAY FCEVDAKKYI | 540 |
| | | | ALAPHVEAES HVEPSFNQED IYITTESLTT TAGRSGTAEH VPSSEIPVPD YTSIHIVQSP | 600 |
| | | | QGLVLNATAL PLPDKEFLSS CGYVSTDQLN KIMP | 634 |
| 40 | GHR | Oreochromis | MALSPSSNLL ILLILSSLDW LPSPGSTELT DWDHTTSSAL IEPHFTECIS RDQETFHCWW | 60 |
| | | niloticus | SPGSFHNLSS PGALRVFYLK KEPPTSQWKE CPEYIHSNRE CFFDEAHTSI WITYCMQLRT | 120 |
| | | | QNNITYFNED DCFTVENIVR PDPPVNLNWT LLNTSPSGLN YDVMINWEPP PTADVRLGWM | 180 |
| | | | RVEYELQYRE RNTTNWEALD IQRQSHQTIY GLRLGKEYEV HIRCRMQAFI KFGEFSESVE | 240 |
| | | | IQVTEIPSTE STVHLTLVLV FGTVGILILI MLIVISQQNR LMIFLLPPVP APKIKGIDSE | 300 |
| | | | LLKKGKLDEL NFMLSGRGMD GLPIYAPDFY QDEPWVELME VDETEDVDNG EKKDNRGSDT | 360 |
| | | | QKLLGQSQPV SQHININCSN SVSGPDAESS QATCYNTDLP EEETLMLMAT LLPGQPDEEE | 420 |
| | | | TSLDTVERSS ASETGERQLI QTQTRGPQTW VNTDFYAQVT NVMPTGGVVL SPGQQLRIQE | 480 |
| | | | SISAAEKETK KKRKESEDSE ESEERKQKEP QFQLLVVDPE GSAYSTESSI QQISTPPPSS | 540 |
| | | | PMPGEGYHII HPQPVEPRPA ATMELNQSPY IIPDSPQFFA PVADYTVVQE LDSHHSLLLN | 600 |
| | | | PPCHQTPPPC LPQHPLKAPM PVGYITPDLL GNLSQ | 635 |
| 41 | IL-6 | Bos taurus | MNSRFTSAFT PFAVSLGLLL VMTSAFPTPG PLGEDFKNDT TPGRLLLTTP EKTEALIKRM | 60 |
| | | | VDKISAMRKE ICEKNDECES SKETLAENKL NLPKMEEKDG CFQSGENQAI CLIRTTAGLL | 120 |
| | | | EYQIYLDYLQ NEYEGNQENV RDLRKNIRTL IQILKQKIAD LITTPATNTD LLEKMQSSNE | 180 |
| | | | WVKNAKIILI LRNLENFLQF SLRAIRMK | 208 |
| 42 | IL-6 | Oreochromis | MPSIFNLHFS SAVMLAALLL CASGAPIEDG SVAGDESGEE TEEAEMSTVK PENIWRSLED | 60 |
| | | niloticus | SAQEYEKAFE HHFQTLENRD QALDSHTPAS IPKHCNITKF RKDACLQTLA KGLLIYSVLL | 120 |
| | | | KHVEKEYRSS LNFSDAPSNI GTLIDLAGMV KGKMKNRNQV TPLTSSEEEQ LLKEVNSPDP | 180 |
| | | | YHRKLHAYSI LRALKAFLSE GKRAVCRMEK RVNQSADTSC | 220 |
| 43 | IL-6R | Bos taurus | MLAVRCALLT ALLAASGTAL VLKGCPALEV ASDVLTSVPG ANVTLTCPGG GPEDNATVHW | 60 |
| | | | VLRDQMTGSH HGRPAGVGRR LLLQSVQLSD SGNYSCYRED HPAGSVRLVV DVPPEKPQIS | 120 |
| | | | CIQKSPLSRV NCEWSPRSPP SLTTKAVLLV KKFPKQVFQE PCQYSPESQR FSCQLAVPEG | 180 |
| | | | DSSFYVLSLC VANSAGNKSS NPLGFDGYKL LQPDPPVNIT VSAVDRNPRW LSVTWQDPPS | 240 |
| | | | WNSYYYRLQF ELRYRTERSE TFTTLMLKDP QYHCIINDAW SGMRHVVQLR AQEEFGNGLW | 300 |
| | | | SEWSPEVTGT PWTESRSSTT TKLPTSTQAP TTTEDNENIF SKDSAKTTSL PVQDSTSVPL | 360 |
| | | | PAFLVAGGSL AFGTLLCIGI ILRFKKTGKL QALKEGKTSA HPSYSLGQLV PERPKPTPAL | 420 |
| | | | VPLISPPVSP SSLESDNTSR NSRPDAKGPG SPYDVSNRDY FFPR | 464 |
| 44 | IL-6R | Oreochromis | MVSAAVLHLC VLAWVASVLA ADDYHLVIAP QSPVVQIGSN FTATCMIINT TEVTADDLYW | 60 |
| | | niloticus | KCSEIIPKEH YIKINTSALN VTVTIRSEKS EWLLCKCKTV SQYVALNEGK FIHGILLRKG | 120 |
| | | | YRPEKPKNLS CIAVHESEKI SSNIRCQWEA VGNQTEAVPT NYTLFVRQTG SDKVYTASIL | 180 |
| | | | GPVNSTTVDM NFFPHHMELE IWVVAQNELG KEESEHLIKL ANCFVKTKPP SVTVISEAVF | 240 |
| | | | PTSLVMNWTS SIDKRYLETI YEIRYCPLGS QMWIYVPLED TSRYIQSFRL QNLTPDTVYV | 300 |
| | | | TQVRCKYNNK NCGYWSDWSR NVTERTPEDL PTSKPDVWII TSAEGINKRQ IEFICKDPEV | 360 |
| | | | SNGRIRRENI TIEKQKYRIR NGTWEIIPVN RSLTDGSSSK KQFTPLKTIR LDDEESVTVY | 420 |
| | | | VTAINSVGAS PQAKLIIYRK AREPPAVEKM EVQFLDGQLL ISWKSPNRTV SEYVVEWVGD | 480 |
| | | | GERNWQRENG KANCSTIKGD LKRFVHYRIS VYPIYGKVVG KPVHEQAYAE QGVPSQAPTV | 540 |
| | | | NLSGDPGCYT AKLEWDEIPL IKRRGFITNY TIYYRSGTET HGIIVPPNTT SYTLTSLSRN | 600 |
| | | | TKYDIWIKAS TIRGSVNGSN HSFTTLKYAP GEIEGIVVGV SLGFLFVVLM TMLLCFCKKD | 660 |
| | | | VIKDNFWPQI PNPGDSTIGN WSPDYPLKAE APRENCLSGI SVLNVDVCDA KCVFEEDKAS | 720 |
| | | | LPLKKDKYLS EEHSSGIGGS SCMSSPRQSV SDSDEGGDMA DTTASTIQYS SVVASSGYKG | 780 |
| | | | QNPSSLPQQS IFSRSESTQP LLDCEENHDT MVQEGSRQCF LRQPCENHNA GNENATDSND | 840 |
| | | | FNPLEMEQQE VLDFCPLEED TEHTAPTDCQ SDDWIPATVS SYMPQLGGYR PQ | 892 |
| 45 | LIF | Bos taurus | MVPAPRAQPA RGPAGWRARQ GAPLPSSSSS SLLNSRRCSS RSALSGIHPP SFLSFSFFPL | 60 |
| | | | SFPTASPAAP WEGRRPPEQL ANSGPGREQV PPPSARAGKL WSVLGVVPLL LVLHWKHGAG | 120 |
| | | | SPLPITPVNA TCATRHPCPS NLMNQIRNQL GQLNSSANSL FILYYTAQGE PFPNNLDKLC | 180 |
| | | | SPNVTDFPPF HANGTEKARL VELYRIIAYL GASLGNITRD QKVLNPYAHG LHSKLSTTAD | 240 |
| | | | VLRGLLSNVL CRLCSKYHVS HVDVTYGPDT SGKDVFQKKK LGCQLLGKYK QVIAVLAQAF | 300 |
| 46 | LIF | Oreochromis | MAHLSLTPTL KLISSGFVWL LWIVLWESTS HTHADTGLTV PQQVTLSPNW GTQELSISWL | 60 |
| | | niloticus | GGGATTFDLI ILRTEFNETV FYETVSPTVN KVSGLHQWTW TSVNPLECTS LSVKIRSRAG | 120 |
| | | | QYTSEWSQTE TLQGHDHPNS EQRVKAFPQD KVLPVGANIT FCCIMDERMF FDKIVYDGNA | 180 |
| | | | TIVTRLSRRT YSTTVSNLVA SRPSGSNMLC LPEDKGKLYG AVVFVGYPPL PTDFKCETYD | 240 |
| | | | LRSAVCQWGK GRDTHLYGIQ RGTRYFVNNR DCTEVNQDVG QKELNCTLDT WEGNWTLVAK | 300 |
| | | | NLLGQYSLTD TAELSHRVCP VAPVQLRVVA DAWNASLSWH WKYDSYSSLA LDCQVEVTAT | 360 |
| | | | EDEKLIKRVF SGVGLRSVFL SDLYPDERYS VKVRCGVQQN FWKWGKWSEL FSFKTKTYVP | 420 |
| | | | DTPDAWIWLN TDNTGTVVWK PLTRRQSHGK LTQYEVFLWS PEENRALAPT VTLPPNTWSM | 480 |
| | | | PVNLTEIAFF SNNSSIVANV TARNDAGSSS PARVHLTSVE PLAASRIVYT DQGFPLFWEN | 540 |
| | | | NANATCGYVV EWLDAFCSQG CPVEWIKLPA GTTNVSLESD TFQPGVRYNL SLYSCSSDSR | 600 |
| | | | ELLKRWQGYA QELVPSVALT LSASQQNADI LLTWNEIPLV NRRGFLLGYN IYNSTGYQFD | 660 |
| | | | LLANLPDPET MSYTAKDFTK GTYKFTVKAY TAAGENTGTT ATVTIEPYAD WLILEILTSL | 720 |
| | | | GLVTLLLAIV TFTCYKKRKW VKKAFYPDIP EPKLPSDWPR TQGPLDVKPS PHSMVCVVET | 780 |
| | | | PEYDCIKEML VAIPEEDEDD EGQGIGDESV DTDEPMSLRY YNQMVDERPI RPRFPDSSAS | 840 |
| | | | SLSSMGSAHT DVTYTGIQTS GSSLVLQLDP QGSTECYQPQ LDQSVSYGGG GYRPQMQPRA | 900 |
| | | | PSDDMGLPSP ESFLEPQAAC SGGYKPQGSW HLDSPVDAAE MGSLAPSLGS PTSVASTQFL | 960 |
| | | | LPDGEENRGE KRQHSSSAAS WLTNLLSSTK P | 991 |
| 47 | TNFa | Bos taurus | MSTKSMIRDV ELAEEVLSEK AGGPQGSRSC LCLSLFSFLL VAGATTLFCL LHFGVIGPQR | 60 |
| | | | EEQSPGGPSI NSPLVQTLRS SSQASSNKPV AHVVADINSP GQLRWWDSYA NALMANGVKL | 120 |
| | | | EDNQLVVPAD GLYLIYSQVL FRGQGCPSTP LELTHTISRI AVSYQTKVNI LSAIKSPCHR | 180 |
| | | | ETPEWAEAKP WYEPIYQGGV FQLEKGDRLS AEINLPDYLD YAESGQVYFG IIAL | 234 |
| 48 | TNFa | Oreochromis | MVAYTTTPVD VEAGPEAKTV VLVEKKSPAE WIWKVCAVLV VVALCLAGVL LFAWYWNTRP | 60 |
| | | niloticus | ERMTQLGQPE ALKAKNTGDK TEPHSTLKRI SSKAKAAIHL EGSDSKGHLE WRNGQGQAFA | 120 |
| | | | QGGFKLEANK IIIPHTGLYF VYSQASERVI CGNTDENEDE EKSLTILSHR IWRYSESMGS | 180 |
| | | | SSTLMSALRS ACQDTIQDSF SDHGWYNAIY LGAVFQLNEG DTLWTETNQL SELETDEGRT | 240 |
| | | | FFGVFAL | 247 |
| 49 | VEGF | Bos taurus | MNFLLSWVHW SLALLLYLHH AKWSQAAPMA EGGQKPHEVV KFMDVYQRSF CRPIETLVDI | 60 |
| | | | FQEYPDEIEF IFKPSCVPLM RCGGCCNDES LECVPTEEFN ITMQIMRIKP HQSQHIGEMS | 120 |
| | | | FLQHNKCECR PKKDKARQEN PCGPCSERRK HLFVQDPQTC KCSCKNTDSR CKARQLELNE | 180 |
| | | | RTCRCDKPRR | 190 |
| 50 | VEGFR | Bos taurus | APESIFDKIY STKSDVWSYG VLLWEIFSLG GSPYPGVQMD EDFCSRLKDG MRMRAPEYAT | 60 |
| | | | PEIYQIMLDC WHKDPKERPR FVELVEKLGD LLQANVQQDG KDYIPLNAIL TGNTAFTYST | 120 |
| | | | PAFSEDFFQE DISAPKENSG SSDNVRYVNA FNF | 153 |
| 51 | FGF2 | Bos taurus | MAAGSITTLP ALPEDGGSGA FPPGHFKDPK RLYCKNGGFF LRIHPDGRVD GVREKSDPHI | 60 |
| | | | KLQLQAEERG VVSIKGVCAN RYLAMKEDGR LLASKCVTDE CFFFERLESN NYNTYRSRKY | 120 |
| | | | SSWYVALKRT GQYKLGPKTG PGQKAILFLP MSAKS | 155 |
| 52 | FGF2 | Oreochromis | MATGGITTLP ATPEDGGSSG FPPGNFKDPK RLYCKNGGFF LRIKSDGGVD GIREKNDPHI | 60 |
| | | niloticus | KLQLQATSVG EVVIKGICAN RYLAMNRDGR LFGARRATDE CYFLERLESN NYNTYRSRKY | 120 |
| | | | PNMYVALKRT GQYKSGSKTG PGQKAILFLP MSAKC | 155 |
| 53 | IGF-1_LR3 | Synthetic | CFRSCDLRRL EMYCAPLKPA KSA CGDRGFYFNK PTGYGSSSRR APQTGIVDEC | 60 |
| | | | MFPAMPLSSL FVNGPRTLCG AELVDALQFV | 83 |
| 54 | TGFbR1 | Bos taurus | MEAAAATPRP RLFLLMLAAA ATLVPEATPL QCFCHLCTKD NFTCVTDGLC FVSVTETTDK | 60 |
| | | | VIHNSMCIAE IDLIPRDRPF VCAPSSKTGS ITTTYCCNQD HCNKIELPTV GKPSSGLGPV | 120 |
| | | | ELAAVIAGPV CFVCISLMLM VYICHNRTVI HHRVPNEEDP SLDRPFISEG TTLKDLIYDM | 180 |
| | | | TTSGSGSGLP LLVQRTIART IVLQESIGKG RFGEVWRGKW RGEEVAVKIF SSREERSWER | 240 |
| | | | EAEIYQTVML RHENILGFIA ADNKDNGTWT QLWLVSDYHE HGSLFDYLNR YTVTVEGMIK | 300 |
| | | | LALSTASGLA HLHMEIVGTQ GKPAIAHRDL KSKNILVKKN GTCCIADLGL AVRHDSATDT | 360 |
| | | | IDIAPNHRVG TKRYMAPEVL DDSINMKHFE SFKRADIYAM GLVFWEVARR CSIGGIHEDY | 420 |
| | | | QLPYYDLVPS DPSVEEMRKV VCEQKLRPNI PNRWQSCEAL RVMAKIMREC WYANGAARLT | 480 |
| | | | ALRIKKTLSQ LSQQEGIKM | 499 |
| 55 | TGFbR1 | Oreochromis | HHRVPNEEDP SMDHPFITVG TTLKDLIYDM TTSGSGSGLP LLVQRTIART IILQESIGKG | 60 |
| | | mossambicus | RFGEVWRGKW RGEEVAVKIF SSREERSWER EAEIYQTVML RHENILGFIA ADNKDNGTWT | 120 |
| | | | QLWLVSDYHE HGSLFDYLNR YTVTVEGMIK LSLSTASGLA HLHMEIVGTQ GKPAIAHRDL | 180 |
| | | | KSKNILVKKN GTCCIADLGL AVRHDSATDT IDIAPNHRVG T | 221 |
| 56 | IR | Bos taurus | ESAGECCSCP KTDSQILKEL EESSFRKTFE DYLHNVVFIP RPSRKRRALG DVGNVTAAVP | 60 |
| | | | TALGLPNTSS TSTPMSSEEH RPFEKVVNXE SLVISGLRHF TGYRIELQAC NQDSPEER | 118 |
| 57 | IR | Oreochromis | FGMVYEGIAK DIVKGEGETR VAVKTVNESA SLRERIEFLN EASVMKAFSC HHVVRLLGVV | 60 |
| | | niloticus | SKGQPTLVVM ELMTHGDLKS FLRSLRPDAE NNPGRPPPTL KEMIQMAAEI ADGMAYLNAK | 120 |
| | | | KFVHRDLAAR NCMVAHDLTV KMGDLE | 146 |
| 58 | ATGL (receptor | Bos taurus | MFPKETTWNI SFAGCGFLGV YHIGVASCLR EHAPFLVANA THIYGASAGA LTATALVTGA | 60 |
| | for PEDF) | | CLGEAGANII EVSKEARKRF LGPLHPSFNM VKTIRGCLLK ILPADCYECA SGRLGISLTR | 120 |
| | | | VSDGENVIIT HENSKEELIQ ANVCSTFIPV YCGLIPPSLQ GVRYVDGGIS DNLPLYELKN | 180 |
| | | | TITVSPFSGE SDICPQDSST NIHELRVTNT SIQFNLRNLY RLSKALFPPE PLVLREMCKQ | 240 |
| | | | GYRDGLRFLR RNGLLNRPNP LLALPPSQPP APEDADAQEG AVAMERTGGK DHLPPPREDH | 300 |
| | | | ILEHLPSRLN EALLEACMEP TDLLTTLSNM LPVRLAMAMM VPYTLPLESA VSFTIRLLEW | 360 |
| | | | LPDVPEDIRW MKEQTGSICQ YLMIRAKRKL GNHLPSRLSG QVVLRRARSL PSVPLSCAAY | 420 |
| | | | SEVLPSWMRN SLSLGDVLAK WEECQRQLLL GLFCTNVAFP PDALRMRVPA GPAPEPPQHP | 480 |
| | | | PSSPPC | 486 |
| 59 | MGF | Synthetic | YQPPSTNKNT KSQRRKGSTF QQRK | 24 |
| | (mechanogrowth | | | |
| | factor) | | | |
| 60 | TGFb1 | Bos taurus | MPPSGLRLLP LLLPLLWLLM LTPGRPVAGL STCKTIDMEL VKRKRIEAIR GQILSKLRLA | 60 |
| | | | SPPSQGDVPP GPLPEAILAL YNSTRDRVAG ESAETEPEPE ADYYAKEVTR VLMVEYGNKI | 120 |
| | | | YDKMKSSSHS IYMFFNTSEL REAVPEPVLL SRADVRLLRL KLKVEQHVEL YQKYSNNSWR | 180 |
| | | | YLSNRLLAPS DSPEWLSFDV TGVVRQWLTR REEIEGFRLS AHCSCDSKDN TLQVDINGES | 240 |
| | | | SGRRGDLATI HGMNRPFLLL MATPLERAQH LHSSRHRRAL DTNYCFSSTE KNCCVRQLYI | 300 |
| | | | DFRKDLGWKW IHEPKGYHAN FCLGPCPYIW SLDTQYSKVL ALYNQHNPGA SAAPCCVPQA | 360 |
| | | | LEPLPIVYYV GRKPKVEQLS NMIVRSCKCS | 390 |
| 61 | TGFb1 | Oreochromis | MKLWFLILMV VYMVGSVHNL STCNTVDLEM VKKKRIEAIR SQILTKLRLQ KAPDEAGEKE | 60 |
| | | niloticus | EIPANLLSLY NSTSEILLEK QDEEQKIIPR EQEEEEYFAK VLNRENMTTK NDTNINYKPK | 120 |
| | | | VISMSFNISE IRSNVGDGLL LTRAELRMLI KEPMILNEER VELYHSQGTS THYLASRFVT | 180 |
| | | | NTLKDKWLSF DVTEPLQTWL QGNENEQKFE LRRYCECGNN DDTLSFSISG TMSRRGDTKD | 240 |
| | | | LQKLNQQLPY IFTMSIPKTN HSKSLRTKRS TDTTDSCGTQ SHNCCLKKLY IDFRKDLGWK | 300 |
| | | | WIHKPTGYYA NYCMGSCTYI WDAENKYSQI LALYKHHNPG ASAQPCCAPQ TLEPLPIIYY | 360 |
| | | | VGRQHKVEQL SNMIVTSCKC S | 381 |
| 62 | TGFb2 | Bos taurus | MHYCVLSAFL LLHLVTVALS LSTCSTLDMD QFMRKRIEAI RGQILSKLKL TSPPEDYPEP | 60 |
| | | | EEVPPEVISI YNSTRDLLQE KASRRAAACE RERSDEEYYA KEVYKIDMPS FLPSENAIPP | 120 |
| | | | TFYRPYFRIV RFDVSSMEKN ASNLVKAEFR VERLQNPKAR VPEQRIELYQ ILKSKDLTSP | 180 |
| | | | TQRYIDSKVV KTRAEGEWLS FDVTDAVHEW LHHKDRNLGF KISLHCPCCT FVPSNNYIIP | 240 |
| | | | NKSEELEARF AGIDGTSTYT SGDQKTIKST RKKNSGKSPH LLLMLLPSYR LESQQSNRRK | 300 |
| | | | KRALDAAYCF RNVQDNCCLR PLYIDFKRDL GWKWIHEPKG YNANFCAGAC PYLWSSDTQH | 360 |
| | | | SRVLSLYNTI NPEASASPCC VSQDLEPLTI LYYIGKTPKI EQLSNMIVKS CKCS | 414 |
| 63 | TGFb2 | Oreochromis | MNVYLVCLFL TLDLATVAFS LSTCSTLDMD QFKKKRIEAI RGQILSKLKL AAPPKDFPEP | 60 |
| | | niloticus | EEVSRDIVSI YNSTRDLLQE KANERAATCE RQRSEEEYYA KEVHKIDMQP VHSPENVISP | 120 |
| | | | TPFNPYFRWL TFDVSSMEKN ASNLVKAELR IFRLQNPGAR VSEQRIELYQ ILGYKGLTSP | 180 |
| | | | TQRYIDSKVI RTQTEGEWLS FDVTEAVSEW LNHRDRNSGF KISLHCPCCT FVPSNNYIIP | 240 |
| | | | NNSEELEARF AGIDDNLIHG RDLKVERKWQ HSVRGPHLLL MLLPSYRLES QSQHKNHRSK | 300 |
| | | | RALDTAYCSK NVQDNCCLRS LYIDFKRDLG WRWIHEPKGY EANFCAGACP YLWSADTQHS | 360 |
| | | | KVLGLYNTIN PEASASPCCV SQDLEPLTIL YYIGKTPKIE QLSNMKN | 407 |
| 68 | EGF | Bos taurus | IDECRRGVHS CGENATCTNM EGNHTCTCAG DLSEPGQICP DSTLLSHLGK NGHNFLKKCF | 60 |
| | | | PEYTPNFEGY CLNGRVCIYF GIANLFSCHC PIGYPGKRGE YIDFDGWDPH SAGRGHQWNT | 120 |
| | | | SPVAVRALVL AFLLLLGLCR AH | 142 |
| 69 | EGFR | Bos taurus | MRLSGAGRAA LLVLLAAHFQ TSLALEEKKV CQGTSNKLTQ LGTFEDHELS LQRMENNCEV | 60 |
| | | | VLGNLEITYM QSSYNLSFLK TIQEVAGYVL IALNTVEKIP LENLQIIRGN VLYENTHALA | 120 |
| | | | VLSNYGANKT GLRELPLRNL QEILQGAVRF SNNPVLCNVE TIQWRDIVNP DFLSNMTGDF | 180 |
| | | | QNQQGNCPKC DPACLNRSCW GAGEENCQKL TKIICAQQCS GRCRGRSPSD CCHNQCAAGC | 240 |
| | | | TGPRESDCLV CRRFRDEATC KDTCPPLMLY DPTTYEMKVN PLGKYSFGAT CVKKCPRNYV | 300 |
| | | | VTDHGSCVRA CSSDSQEVEE DGVRKCKKCD GPCGKVCNGI GIGEFKDTLS INATNIKHFR | 360 |
| | | | NCTSISGDLH ILPVAFRGDS FTRTAPLDPK ELDILRTVKE ITGFLLIQAW PENRTDLHAF | 420 |
| | | | ENLEIIRGRT KQHGQFSLAV VGLDITSLGL RSLKEISDGD VIISGNRNLC YADTIRWKKL | 480 |
| | | | FGTSTQKTKI LNNRSEKQCK AAGHICHPLC SSEGCWGPGP KYCMSCQNFS RGKECVGKCN | 540 |
| | | | ILEGEPREFV ENSECVQCHP ECLPQAMNVT CTGRGPGNCV KCAHYIDGPH CVKTCPAGVA | 600 |
| | | | GENGTLIWKF ADANHVCLLC HPNCTYGCEG PGLEGCPQKG PKIPSIATGI VGGLLLVVVL | 660 |
| | | | ALSVGLFMRR RHIVRKRTLR RLLQERELVE PLTPSGEAPN QALLRILKET EFKKVKVLGS | 720 |
| | | | GAFGTVYKGL WIPEGEKVKI PVAIKELREA TSPKANKEIL DEAYVMASVD NPHVCRLLGI | 780 |
| | | | CLTSTVQLIT QLMPFGCLLD YVREHKDNVG SQYLLNWCVQ IAKGMNYLED RRLVHRDLAA | 840 |
| | | | RNVLVKTPQH VKITDFGLAK LLGAEEKEYH AEGGKVPIKW MALESILHRI YTHQSDVWSY | 900 |
| | | | GVTVWELMTF GSKPYDGIPA SEISTVLEKG ERLPQPPICT IDVYMIMVKC WMIDADSRPK | 960 |
| | | | FRELILEFSK MARDPQRYLV IQGDERMHLP SPTDSNFYRA LMDEEDMEDV VDADEYLVPQ | 1020 |
| | | | QGFFHSPTTS RTPLLSSLST SSNTPTVTCV DRNGSYPLKE DSFLQRYSSD PTGALIEDSM | 1080 |
| | | | DDTFLPVPEY VNQSVPKRPA GSVQNPVYHN QPLYPAPGRD PQYQNSLSNA VDNPEYLNTT | 1140 |
| | | | HPACINGVLD GPALWAQKGS HQFSLDNPDY QQAFFPKEAK SNGIFKGPAA ENAEYLRAAP | 1200 |
| 70 | IGF | Bos taurus | MGKISSLPTQ LFKCCFCDFL KQVKMPITSS SHLFYLALCL LAFTSSATAG PETLCGAELV | 60 |
| | | | DALQFVCGDR GFYFNKPTGY GSSSRRAPQT GIVDECCFRS CDLRRLEMYC APLKPAKSAR | 120 |
| | | | SVRAQRHTDM PKAQKEVHLK NTSRGSAGNK NYRM | 154 |
| 71 | Bone | Homo | MIPGNRMLMV VLLCQVLLGG ASHASLIPET GKKKVAEIQG HAGGRRSGQS HELLRDFEAT | 60 |
| | morphogenic | sapiens | LLQMFGLRRR PQPSKSAVIP DYMRDLYRLQ SGEEEEEQIH STGLEYPERP ASRANTVRSF | 120 |
| | protein 4 | | HHEEHLENIP GTSENSAFRF LENLSSIPEN EVISSAELRL FREQVDQGPD WERGFHRINI | 180 |
| | (BMP4) | | YEVMKPPAEV VPGHLITRLL DTRLVHHNVT RWETFDVSPA VLRWTREKQP NYGLAIEVTH | 240 |
| | | | LHQTRTHQGQ HVRISRSLPQ GSGNWAQLRP LLVTFGHDGR GHALTRRRRA KRSPKHHSQR | 300 |
| | | | ARKKNKNCRR HSLYVDFSDV GWNDWIVAPP GYQAFYCHGD CPFPLADHLN STNHAIVQTL | 360 |
| | | | VNSVNSSIPK ACCVPTELSA ISMLYLDEYD KVVLKNYQEM VVEGCGCR | 408 |
| 72 | Bone | Bos taurus | MIPGNRMLMV VLLCQVLLGG ASHASLIPET GKKKVAEIQG HAGGRRSGQS HELLRDFEAT | 60 |
| | morphogenic | | LLQMFGLRRR PQPSKSAVIP DYMRDLYRLQ SGEEEEEEQI QGIGLEYPER PASRANTVRS | 120 |
| | protein 4 | | FHHEEHLENI PGTSENSAFR FLFNLSSIPE NEVISSAELR LFREQVDQGP DWDQGFHRIN | 180 |
| | (BMP4) | | IYEVMKPPAE VVPGHLITRL LDTRLVHHNV TRWETFDVSP AVLRWTREKQ PNYGLAIEVT | 240 |
| | | | HLHQTRTHQG QHVRISRSLP QGSGDWAQLR PLLVTFGHDG RGHALTRRRR AKRSPKHHPQ | 300 |
| | | | RARKKNKNCR RHSLYVDFSD VGWNDWIVAP PGYQAFYCHG DCPFPLADHL NSTNHAIVQT | 360 |
| | | | LVNSVNSSIP KACCVPTELS AISMLYLDEY DKVVLKNYQE MVVEGCGCR | 409 |

TABLE 3
Additional components for use in the disclosed methods and compositions.

| SEQ | Additional | | |
|---|---|---|---|
| ID | components | | |
| NO: | Protein name | Sequence | |
| 64 | Puromycin | MTEYKPTVRL ATRDDVPRAV RTLAAAFADY PATRHTVDPD RHIERVTELQ ELFLTRVGLD | 60 |
| | resistance | IGKVWVADDG AAVAVWTTPE SVEAGAVFAE IGPRMAELSG SRLAAQQQME GLLAPHRPKE | 120 |
| | | PAWFLATVGV SPDHQGKGLG SAVVLPGVEA AERAGVPAFL ETSAPRNLPF YERLGFTVTA | 180 |
| | | DVEVPEGPRT WCMTRKPGA | 199 |
| 65 | Green fluorescent | MSKGEELFTG VVPILVELDG DVNGHKFSVR GEGEGDATNG KLTLKFICTT GKLPVPWPTL | 60 |
| | protein (GFP) | VTTLTYGVQC FSRYPDHMKR HDFFKSAMPE GYVQERTISF KDDGTYKTRA EVKFEGDTLV | 120 |
| | | NRIELKGIDF KEDGNILGHK LEYNENSHNV YITADKQKNG IKANFKIRHN VEDGSVQLAD | 180 |
| | | HYQQNTPIGD GPVLLPDNHY LSTQSVLSKD PNEKRDHMVL LEFVTAAGIT HGMDELYK | 238 |
| 66 | T2A peptide | GSGEGRGSLL TCGDVEENPG P | 21 |
| 67 | P2A peptide | GSGATNFSLL KQAGDVEENP GP | 22 |

### EXAMPLES

### Example 1—Generating “Self-Sufficient” Cells for Low-Cost Production of Cell-Cultured Foods

The disclosed technology exploits genetic strategies to generate “self-sufficient” cell lines that endogenously produce all of the requisite signaling molecules for growth in low cost, chemically defined cell culture media. Specifically, ectopic expression of growth factors (e.g., fibroblast growth factor (FGF), transforming growth factor (TGF), neuregulin (NRG), Insulin-like growth factor (IGF), etc.), growth factor receptors (FGF receptors, TGF receptors, NRG receptors, IGF receptors, etc.) and signaling/nutrient transport proteins (e.g., insulin, transferrin, etc.) ameliorate the need for the exogenous inclusion of these proteins in cell culture media. As growth factors and signaling proteins contribute over 95% of the cost of standard cell culture media, this invention could drastically lower the cost of production of cultured meats.

In this work, stem cell lines from food-relevant tissues (e.g., muscle, fat, liver, connective tissue) of relevant animal species (e.g., bovine, porcine, piscine, galline) are engineered to express factors comprising the aforementioned genes, constitutively, or under controllable promoter systems. Options for genetic engineering include insertion of cassettes containing these genes, e.g., through CRISPR/Cas9, transposon-mediated, or recombinase-mediated genetic insertion, or by activating the expression of the endogenous genes in cells. In the preliminary work, transposon-mediated insertion of FGF2, TGF-beta3, and NRG1 and their corresponding receptors are demonstrated. However, more directed engineering strategies such as CRISPR/Cas9 also be utilized along with additional proteins, including insulin and transferrin. A full list of relevant potential genes is included in Table 1.

Cultured meat—or meat produced through cell culture and tissue engineering—offers the potential to drastically alter our world's meat production system by addressing the environmental, ethical, and health concerns associated with modern animal agriculture. The high costs of current cell culture media are prohibitive to this effort. Therefore, bringing down the costs of this media is a key hurdle facing cultured meat's development and reaching price parity with conventional meats. The proposed approach offers a promising option, as it completely eliminates the need for the most expensive components of cell culture media, thereby drastically lowering costs.

Endogenous growth factors and signaling molecules have been produced in CHO cells and 3T3 fibroblasts to abrogate the need for these components in cell culture media (DOI: 10.1007/BF00353933; PMID 1325181; U.S. Pat. No. 6,797,515B2). Specifically, these cells were engineered to express insulin or IGF, IGFR, and transferrin. While this has been demonstrated for these proteins and in CHO and 3T3 cells for pharmaceutical applications, the use in food production is novel. Additionally, the growth factors that are most relevant for food-relevant stem cells (e.g., FGF, TGF) are not mentioned or explored in these past examples.

FIG. 1 shows a genetic construct that has been generated and transfected into bovine muscle stem cells.

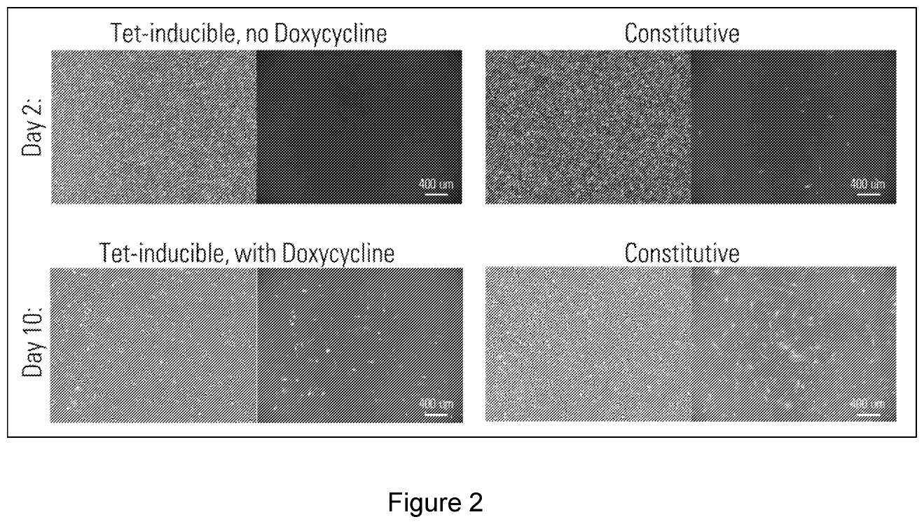

FIG. 2 shows GFP expression in bovine satellite cells that have been transfected with the construct in shown in FIG. 1 as well as the same construct with a TetOn inducible promoter system instead of a constitutive CMV promoter. These constructs were produced and inserted into the genome of bovine satellite cells through Sleeping beauty transposon-mediated transgenesis. In addition, the inventors expect that adding growth factor receptor overexpression, will improve the efficacy of endogenously produced growth factors.

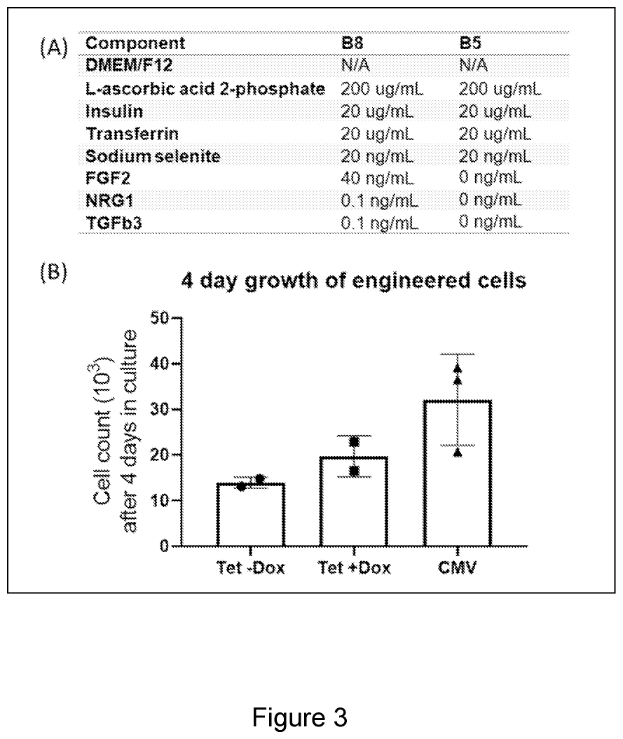

Growth of engineered cells: Engineered bovine satellite cells (shown in FIG. 2) were cultured in a B8-based media without growth factors FGF-2, NRG1, or TGFb3 (called “B5”; FIG. 3)¹. Over four days, cells expressing growth factor genes (i.e., Tet-inducible construct with doxycycline & constitutive CMV construct) showed increased growth compared with cells not expressing growth factor genes (Tet-inducible construct without doxycycline). This is particularly true for constitutive expression, which potentially correlates with the increased expression levels apparent in FIG. 2 (as shown by increased GFP expression & green fluorescence compared with inducible expression). These results suggest that ectopic expression of growth factor genes improves growth in growth-factor-free media by overcoming cellular requirements for exogenous medium factors, and that higher levels of expression may improve outcomes. Current efforts (as before) are exploring combining growth factor expression with growth factor receptor expression in order to amplify signaling pathways and improve outcomes.

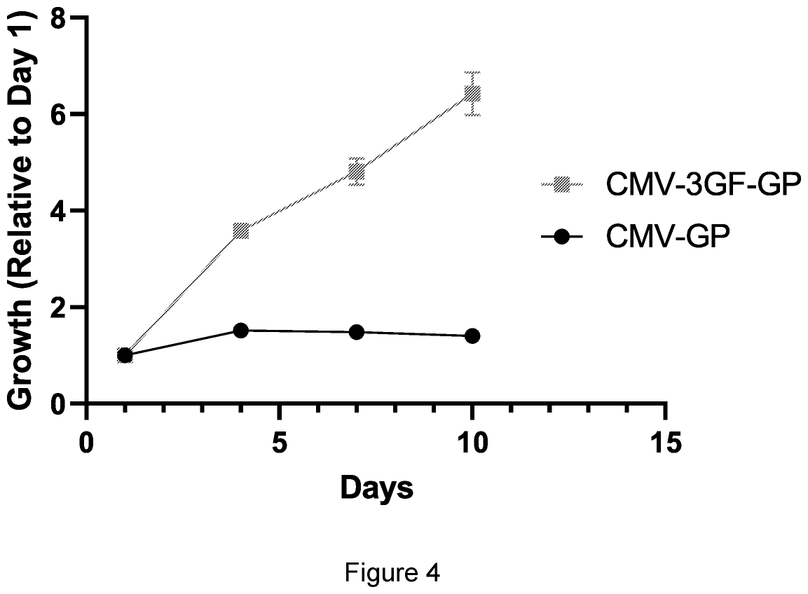

After the data in FIG. 3 was gathered, B8 media was optimized for bovine satellite cell growth by the addition of recombinant albumin². This media is covered in filing T002479 and the referenced publication and has been termed “Beefy-9.” Since Beefy-9 showed significantly improved bovine satellite cell growth compared to B8, growth-factor-producing cells were cultured in Beefy-9 minus all three growth factors (FIG. 4). Results indicate that ectopic growth factor expression significantly improves cell growth over four days.

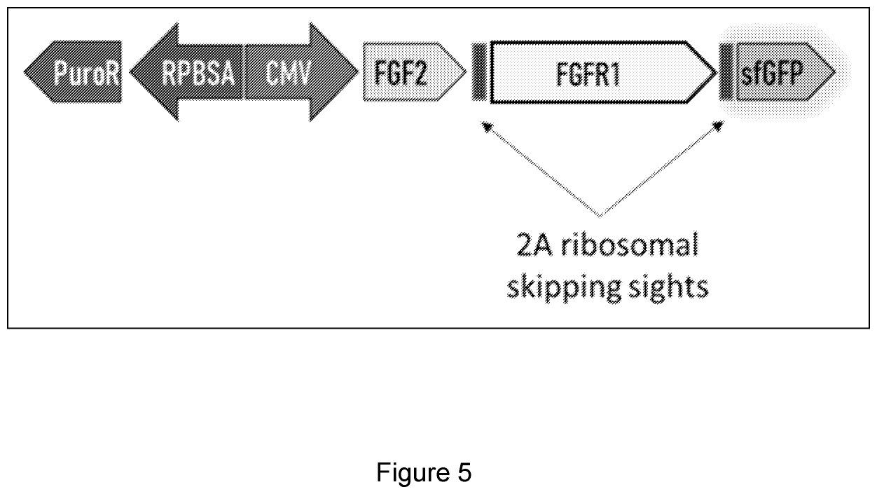

FGF-2 and FGFR1 co-expression: To explore how co-expression of relevant growth factors and growth factor receptors can improve upon results in FIG. 3, we have built additional constructs which contain FGF-2 and its associated receptor, FGFR1, along with a linked GFP (FIG. 5). This system is designed to amplify FGF signal transduction.

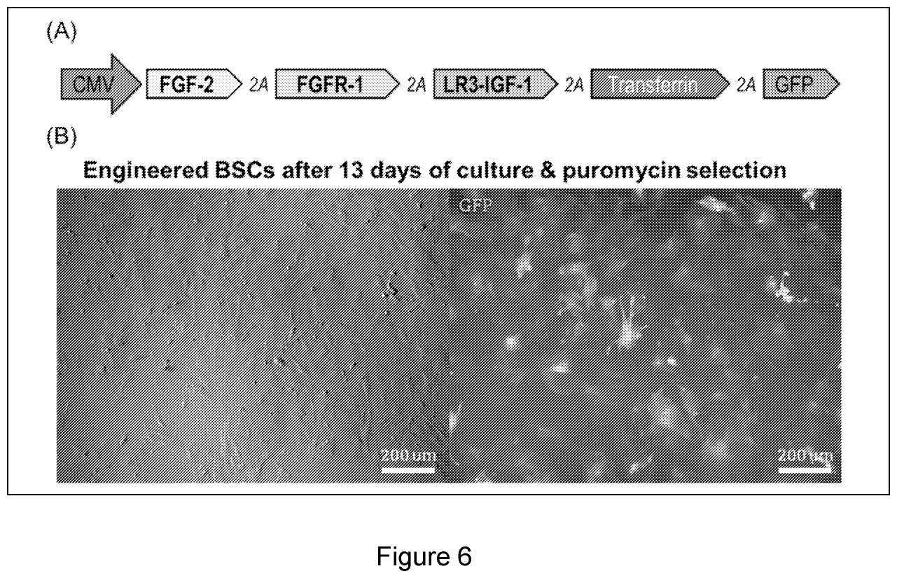

Cells capable of growth in fully protein-free-medium: The ultimate extension of the above-described work is to generate cells which are capable of growing in medium that contains no recombinant protein components. These cells would need to ectopically express, for instance: FGF-2 with or without FGFR1; insulin or insulin-like growth factor (IGF) with or without the insulin/IGF receptor; Transferrin; Albumin (unless albumin were replaced in the media with something such as plant hydrolysates³); and potentially NRG and TGF with or without respective receptors (though it may be the case that these growth factors are less necessary to the media and so could be removed without ectopically expressing them in cells—Supplementary FIG. 1). Ultimately, these cells would be capable of growing in a highly minimal media consisting only of a carbon source (e.g., glucose), amino acids, salts, minerals, trace nutrients (e.g., vitamins), and potentially crude plant hydrolysates (e.g., to replace albumin if not endogenously produced) which would significantly lower the cost of culture media and the resulting price of cultured meat products. We have generated one such construct and demonstrated successful expression in bovine satellite cells (FIG. 6). Current efforts are focused on exploring the efficacy of these constructs using similar experiments to those in FIG. 4.

### Additional Considerations and Applications

The above work uses the Sleeping Beauty transposition system; however, other options include CRISPR/Cas systems, TALENS, ZFNs, viral delivery (e.g., lentivirus), other transposons (e.g., Piggy Bac), gene plasmids, or transient tools (e.g., mRNAs, siRNAs, small molecules, etc.) which are well known in the art.

### REFERENCES

1. Kuo, H., Gao, X., Dekeyser, J., Fetterman, K. A., Pinheiro, E. A., Weddle, C. J., Orman, M. V, Romero-tejeda, M., Jouni, M., Blancard, M., Magdy, T., Epting, C., George, A. L. & Burridge, P. W. Negligible-Cost and Weekend-Free Chemically Defined Human iPSC Culture. Stem Cell Reports 14, 256-270 (2019).
2. Stout, A. J., Mirliani, A. B., White, E. C., Yuen, J. S. K. & Kaplan, D. L. Simple and effective serum-free medium for sustained expansion of bovine satellite cells for cell cultured meat. bioRxiv 2021.05.28.446057 (2021). doi:10.1101/2021.05.28.446057
3. George, F., Kerschen, D., Van Nuffel, A., Rees, J. F. & Donnay, I. Plant protein hydrolysates (plant peptones) as substitutes for animal proteins in embryo culture medium. Reprod. Fertil. Dev. 21, 587-598 (2009).
4. Das, M., Rumsey, J. W., Bhargava, N., Gregory, C., Riedel, L., Kang, J. F., Hickman, J. J., Reidel, L., Kang, J. F. & Hickman, J. J. Developing a novel serum-free cell culture model of skeletal muscle differentiation by systematically studying the role of different growth factors in myotube formation. In Vitro Cell. Dev. Biol. Anim. 45, 378-387 (2009).

1. A modified non-human cell ectopically expressing two or more growth factors or cytokines or receptors thereof that promote cell growth, wherein the two or more factors are selected from neuregulin (NRG), insulin (INS), serotransferrin (TF), fibroblast growth factor 1 (FGF1), fibroblast growth factor receptor 1 (FGFR1), fibroblast growth factor receptor 2 (FGFR2), transforming growth factor beta 1 (TGFb1), transforming growth factor beta 3 (TGFb3), transforming growth factor beta receptor 2 (TGFBR2), insulin-like growth factor (IGF), insulin-like growth factor receptor 1 (IGF1R), platelet-derived growth factor (PDGF), Platelet-derived growth factor receptor alpha subunit (PDGFRa), platelet-derived growth factor receptor beta subunit (PDGFRb), cardiotropin (CT1), leukemia inhibitory factor receptor subunit alpha (LIF1Ra), hepatocyte growth factor (HGF), hepatocyte growth factor receptor (HGFR), epidermal growth factor (EGF), epidermal growth factor receptor (EGFR), pigment-epithelium derived growth factor (PEDF), somatotropin (growth hormone, GH), somatotropin receptor (growth hormone receptor, GHR), interleukin 6 (IL-6), interleukin 6 receptor (IL-6R), leukemia inhibitory factor (LIF), tumor necrosis factor alpha (TNFa), vascular endothelial growth factor (VEGF), vascular endothelial growth factor receptor (VEGFR), IGF-1 LR3, transforming growth factor beta receptor 1 (TGFbR1), insulin receptor (IR), adipose triglyceride lipase (ATGL, receptor for PEDF), mechanogrowth factor (MGF, splice variant of IGF), albumin, and transferrin, or mutated variants of any of the foregoing factors.

2. The modified cell of claim 1, wherein the cell does not require exogenous supplementation with growth factors.

3. The modified non-human cell of claim 1, wherein the cell is capable of growing in media that contains no exogenous recombinant protein components.

4. The modified non-human cell claim 1, wherein the cell comprises three or more factors selected from the list set forth in claim 1.

5. The modified non-human cell claim 1, wherein the cell comprises four or more factors selected from the list set forth in claim 1.

6. The modified non-human cell claim 1, wherein the two or more factors comprise two or more factors selected from fibroblast growth factor (FGF), transforming growth factor (TGF), neuregulin (NRG), insulin, insulin-like growth factor (IGF), FGF-receptor (FGF-R), albumin, transferrin and combinations thereof.

7. The modified non-human cell claim 1, wherein one of the two or more factors comprise one of:
(a) FGF-2, receptor FGFR1 or combination thereof;
(b) transferrin;
(c) insulin, insulin-like growth factor (IGF) or a combination thereof;
(d) recombinant albumin;
(e) NRG and NRG receptor;
(f) TGF and TGF receptor; or
(g) combinations of (a)-(f).

8. The modified non-human cell of any one of claim 1, wherein one of the two or more factors comprise FGF-2, NRG1, and TGF-beta3.

9. The modified non-human cell claim 1, wherein the two or more factors are expressed by one or more exogenous vectors in the cell.

10. (canceled)

11. The modified non-human cell of claim 9, wherein the vector comprises ribosomal skipping sites to express the two or more factors.

12.-14. (canceled)

15. The modified non-human cell of claim 1, wherein the cell is a muscle cell, a fat cell, or a connective tissue cell.

16. The modified non-human cell of claim 1, wherein the cell is a bovine cell, a piscine cell, a porcine cell, or a galline cell.

17. A composition comprising the modified non-human cell of claim 1.

18. A meat product comprising a population of the cells of claim 1.

19.-20. (canceled)

21. A method of producing a meat product in in vitro culture, the method comprising:
culturing a population of the modified non-human cells of claim 1 in minimal culture medium for a sufficient time to increase the number of cells, whereby the method produces a non-human animal tissue suitable for human and/or animal consumption and wherein the minimal media does not contain exogenous growth factors.

22.-25. (canceled)

26. A method of producing a population of modified cells for making a food product, the method comprising:
(a) expressing two or more factors of Table 1 in cells;
(b) culturing the cells of (a) in minimal medium for a sufficient time to promote growth of cells to a sufficient number to produce a food product; wherein the minimal media does not contain exogenous growth factors.

27.-42. (canceled)

43. A population of cells made by the method of claim 26.

44.-45. (canceled)

46. A method of producing a meat product in in vitro culture, the method comprising:
co-culturing a population of target cells and a feeder cell population comprising the modified non-human cells of claim 1 in minimal culture medium for a sufficient time to increase the number of target cells, whereby the method produces a non-human animal tissue suitable for human and/or animal consumption and wherein the minimal media does not contain exogenous growth factors and wherein the target cell is not genetically modified.

47.-53. (canceled)

54. A food product comprising a population of the target cells produced by the method of claim 46.

55. (canceled) 56. The modified non-human cell of claim 1, wherein the two or more factors are expressed by polynucleotide sequences that are integrated into the genome of the cells. 57. The modified non-human cell of claim 1, wherein the two or more factors are expressed by polynucleotide sequences that are not integrated into the genome of the cells.


SEQUENCE LISTING (99 sequences)

SEQ ID NO: 1 - protein, 701 aa, Bos taurus
MEIYSPDMSEGAAERSSSPSTQLSADPSLDGLPAAEDMPETQTEDGRSPGLVGLTLPCCVCLETERLRGCLNSEKICIVPILACLVSLCLCIAGLKWVFVDKIFEYDSPTHLDPGGLGQDPIISLDPTAASAAVWVSAKAYTSPVSRAQSETEVRVTVQVDNALGSSEPSAVPTPKNRIFAFSFRPSTAPSLPSPTRSPDVRTPKAATQPPSTETNLQTAPKLSTSTSTAGTSHLVKCAEKEKTFCVNGGECFMVKDLSNPSRYLCKCPNEFTGDRCQNYVMASFYKHLGIEFMEAEELYQKRVLTITGICIALLVVGIMCVVAYCKTKKQRKKLHDRLRQSLRSERNTMMNVANGPHHPNPPPENVQLVNQYVSKNVISSEHIVEREAETSFSTSHYTSTAHHSTTVTQTPSHSWSNGHTESIISESHSVIVMSSVENSRHSSPTGGPRGRLNGLGGPRECNSFLRHARETPDSYRDSPHSERYVSAMTTPARMSPVDFHTPSSPKSPPSEMSPPVSSTTVSMPSMAVSPFVEEERPLLLVTPPRLREKYDHHAQQFNSFHCNPAHESNSLPPSPLRIVEDEEYETTQEYEPAQEPVKKLTNSSRRAKRTKPNGHIAHRLEMDNNTGADSSNSESETEDERVGEDTPFLAIQNPLAASLEAAPAFRLVDSRTNPTGRFSPQEELQARLSGVIANQDPIAV

SEQ ID NO: 2 - protein, 649 aa, Oreochromis niloticus
MEEVSAGPAPPFGSVSPTGSTVDTRQVPEEKPEETEAAGESGEEAIEGTAEGGGAGDDGEHVGAFGIVTLPEACCVCIEMEQINNCLHSEKICILPILACLLSLALCTAGLKWVFVDKIFEYEPPSHLDPKPIGQDPIIIPVEPTLGITVSFPHTTPSNNSLTTTFTLTPEHPEVSVKDKSTQHHHQTICKCLFPATISTSTTAKTSSHVTRCSDSQRNYCVNGGECFTLEIIPGSTKCPVEFTGDRCQNYVMASFYKWLSFFSEAEELYQKRILTITGICIALLVVGIMCVVAYCKTKKQRKKLRDRLRQSLRNKRKNTSTGIDSNVAKSTTGRPNSNLPLQDLQLIGVCCYFVSVVSHTAEKETETNFSTSKYTLSVHDPTTLTNISSQSWSNDWSNSVQSDTESVSVMSLAENSQRATQGARGRLNATGGTSDLSAHSKKFVPVMTTLIQLTSESQNSLATPGSPPSEISAPISSLAISVPSVTLSPSGEEERPLLHQLHKSSSRDVQKRTSSHYNHGHVADSLPSSPLFTMENADYQTIQDTRAASCMFSTAPVKLINTNNNVSDCSNKSVTDHVVDYLRLNGDSILVSDTEEPRGEHIPFLSADRNTALLLRAIDSSRTNPASPNDDLPVKSSSLINQQDSIAA

SEQ ID NO: 3 - protein, 105 aa, Bos taurus
MALWTRLRPLLALLALWPPPPARAFVNQHLCGSHLVEALYLVCGERGFFYTPKARREVEGPQVGALELAGGPGAGGLEGPPQKRGIVEQCCASVCSLYQLENYCN

SEQ ID NO: 4 - protein, 113 aa, Oreochromis niloticus
MAALWLQAFSLLVLMMVSWPGSQAVGGPQHLCGSHLVDALYLVCGDRGFFYNPRRDVDPLLGFLPPKAGGAVVQGGENEVTFKDQMEMMVKRGIVEECCHKPCTIFDLQNYCN

SEQ ID NO: 5 - protein, 704 aa, Bos taurus
MRPAVRALLACAVLGLCLADPERTVRWCTISTHEANKCASFRENVLRILESGPFVSCVKKTSHMDCIKAISNNEADAVTLDGGLVYEAGLKPNNLKPVVAEFHGTKDNPQTHYYAVAVVKKDTDFKLNELRGKKSCHTGLGRSAGWNIPMAKLYKELPDPQESIQRAAANFFSASCVPCADQSSFPKLCQLCAGKGTDKCACSNHEPYFGYSGAFKCLMEGAGDVAFVKHSTVFDNLPNPEDRKNYELLCGDNTRKSVDDYQECYLAMVPSHAVVARTVGGKEDVIWELLNHAQEHFGKDKPDNFQLFQSPHGKDLLFKDSADGFLKIPSKMDFELYLGYEYVTALQNLRESKPPDSSKDECMVKWCAIGHQERTKCDRWSGFSGGAIECETAENTEECIAKIMKGEADAMSLDGGYLYIAGKCGLVPVLAENYKTEGESCKNTPEKGYLAVAVVKTSDANINWNNLKDKKSCHTAVDRTAGWNIPMGLLYSKINNCKFDEFFSAGCAPGSPRNSSLCALCIGSEKGTGKECVPNSNERYYGYTGAFRCLVEKGDVAFVKDQTVIQNTDGNNNEAWAKNLKKENFEVLCKDGTRKPVTDAENCHLARGPNHAVVSRKDKATCVEKILNKQQDDFGKSVTDCTSNFCLFQSNSKDLLFRDDTKCLASIAKKTYDSYLGDDYVRAMTNLRQCSTSKLLEACTFHKP

SEQ ID NO: 6 - protein, 718 aa, Oreochromis niloticus
VAMWRTVGTLLLILHTVFGQSTIKWCTISQNEQRKCQAMSQAFSTASIRPSLSCVNAASVEDCGQKLQRKEADALSMSAKDIYNLGKTVSLKMAGSESQAGSVTTYYAVAVVKKENSGININNLAGKKSCHTGIGRTVGWNMPIGYLIDQGYMSVMGCNIPQGVANFFSASCIPGATQGEPQSLCQLCRGDESGQHKCEMSSNERYYSYEGAFRCLADGAGEVAFTKDTTVEENTDGRGPTWAKDLKSSDYELLCPDGTRAPVTQWSRCNLVPVPSRGVVVRNDISPSVVFNMLMEGLEKSNFPMFSSAGYGDGTVLFSNLTTTFKAANSDEPKKWMGERYHNALKAMDCDPKDIPNALRWCVISSGEQQKCADMSVAFQSKGLTPNINCVYGDSVTHCMKKIKDNDADAITLDGGYIYTAGKDYGLVPATGESYTDDHDGSSYYAVAVVKKSSFDIRNLDDLRGRRSCHTGYGRTAGWNIPVAVLMERGLISPQQCQIPQAVGDFFEKCCVPGANQAGFPSNLCELCVGDESGQNKCEKGKDLYDGYDGAFRCVAKGEGDVAFVKHSTVLENTAGKTSLPPSDFQLLCQSGGKAEATHYKYCNLGRVPSHAVMVRPDMNIHAIYGLLDQAQTYFGSDTGTAFKMFDSQVYKGTDLIFKDSTVRLVGVADRKTYQEWLGQIYLDSLVDLECSSSNAVASSVWLLPTALFSFMLTNYWM

SEQ ID NO: 7 - protein, 465 aa, Bos taurus
MAEGETTTFTALTEKFNLPLGNYKKPKLLYCSNGGYFLRILPDGTVDGTKDRSDQHIMAEGETTTFTALTEKFNLPLGNYKKPKLLYCSNGGYFLRILPDGTVDGTKDRSDQHIQLQLCAESIGEVYIKSTETGQFLAMDTDGLLYGSQTPNEECLFLERLEENHYNTYISKKHAEKHWFVGLKKNGRSKLGPRTHFGQKAILFLPLPVSSDMAEGETTTFTALTEKFNLPLGNYKKPKLLYCSNGGYFLRILPDGTVDGTKDRSDQHIQLQLCAESIGEVYIKSTETGQFLAMDTDGLLYGSQTPNEECLFLERLEENHYNTYISKKHAEKHWFVGLKKNGRSKLGPRTHFGQKAILFLPLPVSSDQLQLCAESIGEVYIKSTETGQFLAMDTDGLLYGSQTPNEECLFLERLEENHYNTYISKKHAEKHWFVGLKKNGRSKLGPRTHFGQKAILFLPLPVSSD

SEQ ID NO: 8 - protein, 207 aa, Oreochromis niloticus
MRPIPSRLSCVFLHLFALFYYAQVTNQSPPNFTQHVSEQSKVTDRTSRRLIRIYQLYSRTSGKHVQVLPNKKINAMAEDGDVHAKLIVETDTFGSRVRIRGAETGLYICMNKRGKLIGKKNGQGRDCIFTEIVLENNYTALKNVRYEGWYMAFTRQGRPRKGSRTRQHQREVHFMKRLPKGHQPTHPSHHQPFDFIHYPFSQRTKRT

SEQ ID NO: 9 - protein, 832 aa, Bos taurus
MWSRKCLLFWAVLVTATLCTAKPAPTLPEQAQPWGAPVEVESLLVHPGDLLQLRCRLRDDVQSINWLRDGVQLADSNRTRITGEEVEVRGSVPADSGLYACVTSSPSGSDTTYFSVNVSDALPSSEDDDDDDDSSSEEKETDNTKPNRMPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKHSGINSSDAEVLTLFNVTEAQSGEYVCKVSNYIGEANQSAWLTVTRPVAKALEERPAVMTSPLYLEIIIYCTGAFLISCMVGSVIIYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDRPNRVTKVAVKMLKSDATEKDLSDLISEMEMMKMIGKHKNIINLLGACTQDGPLYVIVEYASKGNLREYLQARRPPGLEYCYNPSHHPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRRDYKDDDDK

SEQ ID NO: 10 - protein, 837 aa, Oreochromis niloticus
MSQTPEWSSRRTKANSRSSVSRMLMRPSILLFLALFAQVLRTQCRPANADEVSVETQAELYTLSIGDRLDLSCCAKDYLHAVNWTKDHVAVVDGEHTRIRNGQLEIESVELTDSGLYTCTTFGNHSIFFNVTVHISASSEDDDDGEESSSEENKLLGSQKLMPMAPQWAHPEKMEKKLHAVPASKTVKFKCQASGNPTPTLKWYKNGKEFKRDHRIGGFKVRDHMWTIIMESVVPSDKGNYTCVVENKYGSINHTYQLDVVERSPHRPILQAGLPANRTVVVGSDVEFECKVFSDPQPHIQWLKHIEVNGNRTGPDGLPYVRVLKHSGVNSSDAQVLTLYNVTEEESGEYICKVSNYIGEVSQSAWLTVIRYEPTAPPHYSPASHTYLEVVIYCVGFFFIFAIMIAIAIIVKIRTTSSKKSDFNSQLAVHKLAKSIPLRRQVTVSVDSSSSIHSGVMLVRPSRLSSSGSPMLSGVSECELPQDPRWELPRDRLVLGKPLGEGCFGQVVMGEAVGLDKEKPNRVTKVAVKMLKADATEKDLSDLISEMEMMKIIGKHKNIINLLGACTQDGPLYVIVEYASKGNLREYLRARRPPGMEYCSNPDQVPVENVSIKDLVSFAYQVARGMEYLASKKCIHRDLAARNVLVTDDSVMKIADFGLARDIHHIDYYKKTTNGRLPVKWMAPEALFDRIYTHQSDVWSFGVLLWEIFTLGGSPYPGVPVEELFKLLKEGHRMDKPSTCTHELYMMMRDCWHAVPSQRPTFKQLVEDLDRALAMTSNQEYLELSVPLDQYSPSYPDTRSSTCSSGEDSVFSHDAGAEEPCLPKFPPHSNGAAIKKR

SEQ ID NO: 11 - protein, 785 aa, Bos taurus
MVSWGRFLCLVVVTMATLSLARPSFNLVDDTTVEPEEPPTKYQISQPEVYVAAPRESLELRCLLRDAAMISWTKDGVHLGPNNRTVLIGEYLQIKGATPRDSGLYACTAARNVDSETVYFMVNVTDAISSGDDEDDADGSEDFVSENSNSKRAPYWTNTEKMEKRLHAVPAANTVKFRCPAGGNPTPTMRWLKNGKEFKQEHRIGGYKVRNQHWSLIMESVVPSDKGNYTCVVENDYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPVREKEIPASPDYLEIAIYCIGVFFIACMVVTVILCRMRNTTKKPDFSSQPAVHKLTKRIPLRRQVSAESSSSMNSNTPLVRITTRLSSTADTPMLAGVSEYELPEDPKWEFPRDKLTLGKPLGEGCFGQVVMAEAVGIDKEKPKEAVTVAVKMLKDDATEKDLSDLVSEMEMMKMIGKHKNIINLLGACTQDGPLYVIVEYASKGNLREYLRARRPPGMEYSYDINRVPEEQMAFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEPYSPCYPDPR

SEQ ID NO: 12 - protein, 390 aa, Bos taurus
MPPSGLRLLPLLLPLLWLLMLTPGRPVAGLSTCKTIDMELVKRKRIEAIRGQILSKLRLASPPSQGDVPPGPLPEAILALYNSTRDRVAGESAETEPEPEADYYAKEVTRVLMVEYGNKIYDKMKSSSHSIYMFFNTSELREAVPEPVLLSRADVRLLRLKLKVEQHVELYQKYSNNSWRYLSNRLLAPSDSPEWLSFDVTGVVRQWLTRREEIEGFRLSAHCSCDSKDNTLQVDINGFSSGRRGDLATIHGMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 13 - protein, 405 aa, Oreochromis niloticus
MNLGKALLFFLLSNCVTVTFLLSTCTTVDIKHIKKKRVEAVRGQILSKLRLTSPPQTTGPSQVPFQVLALYNSTKELIEELGKDRQQSCGQDNTETEYYAKEIYKFNMINGPQENNDLPNCPRGITPKIFRFNVSIMEKNATNLFRAEFRALRIPNPSARRNEQRIELYQILQKDDPKAKQRYIGGKNVLTKGTPEWVSFDVTETVREWLMNRRTNLGLEISVHCPCHTFRPNGDIIENANEVLEVKFKGMEAEYDEQSRVKSQKEQLYPHLILMMLPPHRLDAQSSSRRRKRALDTNYCFNNYEENCCVRQLYINFREDLGWRWIHQPEGYYANFCSGPCPYLRSADTTHSSLLSLYNTLNPEASASPCCVPQDLEPLTILYYVGRSPKVEQLSNMVVKSCKCS

SEQ ID NO: 14 - protein, 499 aa, Bos taurus
MEAAAATPRPRLFLLMLAAAATLVPEATPLQCFCHLCTKDNFTCVTDGLCFVSVTETTDKVIHNSMCIAEIDLIPRDRPFVCAPSSKTGSITTTYCCNQDHCNKIELPTVGKPSSGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEVARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 15 - protein, 452 aa, Bos taurus
MHLLAKPQSSGSREAAWFSSLLLHDCRGLLLPGLAAFLPGPRLKMHLQRALVVLALLNFATVSLSMSTCTTLDFNHIKRKRVEAIRGQILSKLRLTSPPDPSGLASVPIQVLDLYNSTRELLEEVHGERGDVCTQANTESEYYAKEIYKFDMIQGLEEHNDLTVCPKGITSKIFRFNVSSVEKNETNLFRAEFRVFRMPNPASKRSEQRIELFQILQPGEHIAKQRYIDGKNLPTRGTGEWLSFDVTDTVREWLLRRESNLGLEISIHCPCHTFQPNGDILENIQELMEIKFKGVDSDDDPGRGDLGRLKKKKEHIPHLILMMIPPNRLDSPGHSQRKKRALDTNYCFRNLEENCCVRPLYIDFRQDLGWKWVHEPKGYYANFCSGPCPYLRSSDTTHSTVLGLYNTLNPEASASPCCVPQDLEPLTILYYVGRTPKVEQLSNMVVKSCKCS

SEQ ID NO: 16 - protein, 405 aa, Oreochromis niloticus; identical to SEQ ID NO: 13

SEQ ID NO: 17 - protein, 588 aa, Oreochromis niloticus
MKARRCSLTFRGVLSAFLISVLLEPGAAGLDVFQLHELCKFCDVEPTSCNGTGICKTDCSISSICEKPTEVCVSIWRRNETNLSIETLCHNPAEPLYGITLDDYNSTTCVMKEKNTTTGLAHFCSCKGAECNEELIFSQTVDPTPDPLESNDSLMSVVLVSMLPLLVMVIAFFGMFYWCRIHRQRRPNRQWESSFNFKRKPIAGGLDCSDACAIMTDDDKSDSSSTHANSLNHNTEPLPIELDLLVGKGRFAQVYKAKLKQTTWNQFETVAVKIFPYEEYASWKNEKDIFSNTDLRHENILHFLTAEERKVEKQYWLITAFHPRGNLQEYLTHHVISWQELQVLSSSLARGVAHLHSDRLPCGRPKVPIVHRDLKSSNILVKNDGTCCVCDFGLGLCLDSSLSVDDLANSGQVGTARYMAPEVLEARLNLENIESFKQTDIYSMALVLWEMTSRCEAIGEVKDYEPAYGSKVREHPCVESMKDNVLRDRGRPEIPDSWLRHQGMAVMCATIKECWDHDPEARLTAHCIAERISELEDEMGKLSSRSSSAEKIVEELKIPIEVEIPEEEVKITEIQNIIAVDCSISDKK

SEQ ID NO: 18 - protein, 832 aa, Bos taurus; identical to SEQ ID NO: 9

SEQ ID NO: 19 - protein, 171 aa, Oreochromis niloticus
MSSAFSFQWHLCDVFKSAMCCISCSHTLSLLLCVLTLTPTATGAGPETLCGAELVDTLQFVCGERGFYFNKPTGYGPSARRSRGIVDECCFQSCELQRLEMYCAPVKTPKISRSVRSQRHTDMPRAPKVSSRANKGTERRTAPQPDKTKNKKEVHQKNSSRGSSGGRNYRM

SEQ ID NO: 20 - protein, 640 aa, Bos taurus
NAIFVPRPERKRREVMQIANTTMSSRSRNTTVLDTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTTYENFIHLMIALPIAVLLIVGGLVIMLYVFHRKRNSSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTHSDVWSFGVVLWEIATLAEQPYQGLSNEQVLRFVMEGGLLDKPDNCPDMLFELMRMCWQYNPKMRPSFLEIISSVKDEMEAGFREVSFYYSEENKPPEPEELDLEPENMESVPLDPSASSASLPLPDRHSGHKAENGPGPGVLVLRASFDERQPYAHMNGGRKNERALPLPQSSTC

SEQ ID NO: 21 - protein, 148 aa, Oreochromis niloticus
FGMVYEGIAKGVVKDEPETRVAIKTVNESASMRERIEFLNEASVMREFNCHHVVRLLGVVSQGQPTLVIMELMTRGDLKSYLRSLRPKEQQWSSLSLPPLKKMLQMAGQIADGMAYLNANKFVHRDLAARNCMVAEDFTVKIGDFGMT

SEQ ID NO: 22 - protein, 211 aa, Bos taurus
MRTWACLLLLGCGYLANALAEEAEIPREVIERLAHSQIHSIRDLQRLLEIDSVGAEEPLETSLRAHGGHGAKHALEKRPVPIRRKRSIEEAIPAVCKTRTVIYEIPRSQVDPTSANFLIWPPCVEVKRCTGCCNTSSVKCQPSRVHHRNVKVAKVEYFRKKAKLKEVQVRLEEHLECTCTSASPSPDHREEEAGRRRESGKKRKRKRLKPT

SEQ ID NO: 23 - protein, 199 aa, Oreochromis niloticus
MRAAVYHILAGYLCVLRTVAQESPLPQELLERLSRSEIRSISDLQRLLEIDSVENEVSEETKHSYHKDVSHSFPDLKRAHTHTRHRRSAMVEEALPAVCKVRTAIYEIPRSQVDPTSANFIIWPPCVEVKRCTGCCNTSNMRCQAARKHYRTVKVAKVEYVRRRPKLREVQVRLEDHLECMCTNKRHISPNSDTDNGIR

SEQ ID NO: 24 - protein, 1098 aa, Bos taurus
MGTSHWALLVLGCLFTGPSLILCQLSLPSILPNENERVVQLNSSFSLRCFGESEVSWQYPMSEEEYPDVEIRNEENNSGLFVTVLEVVSASAAHTGLYTCYYNHTQMDENEIEGRHIYIYVPDPDVAFVPLGMTDSLVIVEDEDSVIIPCRTTDPETPVTLLSSEGIVPASYDSRQGFKGTFSVGLYICEATVRGKKFQTIPFNVYAYTATSKLDLEMESPKTVYKAGESIVVTCTVFNNEVVDFQWTYPGQMKGKGITRLEETKFPSIKLVYTLRVPEATVKDSGDYECAAHQATKEVKKMRNVTISVHEKGFIEIKPNFNLLEAVNLHEVKHFVVDVQAYPPPKITWLKDNLTLIENLTEITTDIEKIQEISYRSKLKLIRAKEEDSGHYTIVVQNEDDVKSYTFELLTQVPSSILDLVDDHHGSNGGQTVRCTAEGTPLPDIEWMVCKDIKKCNNETSWTVLANNVSNIITGVHPRDRSTVEGQVTFAKVEETIAVRCLAKNLLGVESRELKLVAPTLRSELTVAAAVLVLLVIVIISLIVLVVIWKQKPRYEIRWRVIESISPDGHEYIYVDPMQLPYDSRWEFPRDGLVLGRILGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRALYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHRIFLSLPHSVDYIKIFKQKHIASEKAMASYSSTLAWKIPWMEEPGRLQSMGLLRVRHD

SEQ ID NO: 25 - protein, 1066 aa, Oreochromis niloticus
KYSLFMTHCMCLLSAPTAGMIPSSSVPLLLPKLDEMVVTLHTPFTLTCHGQENLGWETPIDVSEQTQEDPNGLFISTITVDSATASHTGYYTCFYSRTFAEDTAEFSRIYIYVPDPDVPFLESLIPFGYHVLSGYDEMEIQCRVSDPNANVTLINSDTQQPVPSIYDSKRGALGIFTAGTYVCKAVINGHEFYSEEYIVHGSTDKDTNELDVELTAKKTALLVGDTITVTCVARGSEILEDHWKYPGKLANRGTKTVRENKRTQEIFYTLTIPQASTKDSGIYSCSITDILSNTSEFLSVKPEFREYESAALDEVREFKAVVNSFPSVHVSWFKDGYPLSDVTAEISTSLQQTSEISYMSVLTLIRAKEEDSGNYTMRVENRNQSRDISLILEVKVPAVIVDLMDVHHGSAQGQSVVCITRGQPTPMVEWFVCKNIKHCANDSSSWVPIPANSTEITLDSHIDEDNNLESQVMFGHLESTMAVRCLARNEMAVVSREVKLVSNGPHPELTVAAAVLVLLVIVIISLIVLVVIWKQKPRYEIRWRVIESVSPDGHEYIYVDPMQLPYDSRWEFPRDRLVLGRILGSGAFGKVVEGTAYGLSRSQPVMKVAVKMLKPTARSSEKQALMSELKIMTHLGPHLNIVNLLGACTKSGPIYIITEYCFYGDLVNYLHKNRENFLSLNPEKNKKELDIFGINPADESSRSYVILSFESKGDYMDMKQADSTQYVPMLEMSNASKYSDIQRSNYDHPPSQKGSSEMEIMLSDDVNEGLTTTDLLSFTYQVAKGMEFLASKNCVHRDLAARNVLLSQGKIVKICDFGLARDIMHDNNYVSKGSTFLPVKWMAPESIFDNLYTTLSDVWSYGILLWEIFSLGGTPYPGMVVDSSFYNKIKSGYRMSKPEHAPHDVYEMMMKCWNSEPEKRPSFLGLSETVASLLPSSYKRQYERVNHEFLKSDHPAVTRVCMENEDYIGIAYKNQGKLKDRESGFDEQRLSSDSGYIIPLPDLDPISDEEYGKRNRHSSQTSEESAIETGSSSSTFAKREGETLEDITLLDEMCLDCSDLVEDSFL

SEQ ID NO: 26 - protein, 1107 aa, Bos taurus
MKDTMWLPSAMQAVVLKGRVLLLPLLFLLGPQASWGLAIIPPGPELVLNLSSTFVLTCSGPAPVVWERMSQKPPQEMTETQDGTFSSVLTLVNVTGLDTGEYFCGYKRSHGLEASERKRLYIFVPDPSVGFLPVDPEELFIFLTEITETTIPCRVTDPRLVVMLHEKKVDVPLPIAYDPQRGFSGTFEDKTYICKTTIGDREVDSDAYHVYSLQVSSINVSVNAVQTVVRQGENITIMCIVTGNEVVNFEWTYPRMESGRLVEPVTDFLFEMPHIRSILHIPSAELGDSGTYICNVSESVSDHRDEKAINVTVVENGYVRLLGELDPVQFAELHRSRTLQVVFEAYPPPTVMWFKDNRTLGDSSAGEIVLSTRNVSETRYVSELTLVRVKVAEAGYYTMRAFHEDAEAQLSFQLQVNVPVRVLELSESHPANGEQTVRCRGRGMPQPHLTWSTCSDLKRCPRELPPTPLGNSSEEESQLETNVTYWAEEQEFEVVSTLRLRHVDQPLSVRCTLHNLLGYDVQEVTVVPHSLPFKVVVISAILALVVLTIISLIILIMLWQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQRCSDKRRPPSAELYSNALPVGLPLPSHVSLPGESDGGYMDMSKDESVDYVPMLDMKGDVKYADIESSNYMAPYDNYVPSAPERTCRVTLINESPVLSYTDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFNGLRSPLDSSSVLYTAVQPNEGDNDYIIPLPDPKPEVADEGPLEGSPSLASSTLNEVNTSSTISCDSPLEPQEEPEPEPQPASQVEPEPKWPPDSSCPGPRAEAEDSFL

SEQ ID NO: 27 - protein, 295 aa, Bos taurus
GPPPWPLKHPALSSPRQEEGPAHSCPAQLRPLLPSPSPPPSNKLSPKSSRGRRSEPETSPPIGPQGCLQGPDTPSLRSLSTLQTTCCPPQVPFATRPSEQREDPQADSSASPLPHLEAKIQQTHSLARLLTKYAEQLLQEYVQHQGDPFGLPGFSPPRLPVADLSDPAPGHAGLPVPERLRLDAAALAALPPLLDVVRRLQGELNPRALRLLRRLEDAARQVRALGAAVEAVLAALGAESRGPRPEPAAAAVAASTASAGVFPAKVLGFRVCGLYLEWVSRTEADLGQLAPGGPA

SEQ ID NO: 28 - protein, 1095 aa, Bos taurus
MMDISLCVKRPSWLVDRKRMRMASNSQWLLLTAVLLYLTNQVNSQKKSTPHDLKCVTNNLKVWDCSWRALFGAGRAASYEVCIEKRSRTCYLLEKNTTIPPLLPGHYEITVHPLYEFGSSKSKFILNEKNISLIPETPEILNLSADFSTSTLHLKWNDKGSVFPHYSNVIWEIKVLHKKNMEIVKLVTYSTTLNGRDTIHHWNWTSDMPLECAIHYVGIRCYIDDPQFSGHKEWSDWSLLKNISLPPDSQTKVFPQDKVILAGSDITVCCVTQEKVLSAQIGSTHCPLIHLDGENVAIRIRNISASASSGTNVVFSVEDNIFGTVVFAGYPPDIPQKLNCETYDLKEIICTWNPGRPTSLEGPRSTSYTLFESFSGKYVRFKGDEVSANENYQLFLQILSSQEIHNFTLNAQNLLGQTESTLLVNITEKVHPRIPTSLKAKDINSTAVILSWHLPGNFTKVKLLCQIEINKTNSEHELRNVTMRGVESSNYLTTVDKLNPYTIYTFRIRCSTDPFWKWSKWSNEKQCLTTEAIPSKGPDIWREWSSDGRNLIIYWKPLPINEAYGKILSYNVSYSSDEETKSLSEIPDTQHRAELQLDKNDYIISVVAKNSAGSSPPSKIASMEIPNDDLKIEQALGMGNRILLTWNYDPNMTCDYVIKWCNSSQSGPCLMDWKKVPPNSTETVIESDQFRPGVRYNFSVYGCRNQGYQLLRSVIGYVEELAPAVAPNFTVEDTSADSILVKWEEIPVEELRGFLRGYLFYFEKGERDTSKIMGLEPGRFDTKVKNITDISQKTLRIADLQGKTSYHLVLRAYTGGGMGPERSMFVVTKENSEGLIIAILIPVAVAVIIGVVTSILCYRKREWIKETFYPDIPNPENCKALQFQKSVCEGNSALKTLEMNPCTPNNVEVLETRSVALKIEDTEIISPVAERPEDRSDAEPENHVVVSYCPPVIEEELANPGAEGGGASQVIYIDVQSMYQPQAKPEGELDSDPVGGAGYKPQMHLPVTSTVDDLDADDDLDKTAGYRPQANVNTWNLVSPDSPRSTDSNSEIVSFGSPCSINSRQFLIPPKDEDSPKSSGGGWSFTNFFQNKPND

SEQ ID NO: 29 - protein, 730 aa, Bos taurus
MWVTRLLPVLLLQHVLLHLLLLPIAIPYAEGQKKRRNTLHEFKRSAKTTLIKEDPLLKIKTKKMNTADQCANRCIRNKGLPFTCKAFVFDKARKRCLWFPFNSMSSGVKKEFGHEFDLYENKDYIRNCIIGKGGSYKGTVSITKSGIKCQPWNSMIPHEHSFLPSSYRGKDLQENYCRNPRGEEGGPWCFTSNPEVRYEVCDIPQCSEVECMTCNGESYRGPMDHTETGKICQRWDHQTPHRHKFLPERYPDKGFDDNYCRNPDGKPRPWCYTLDPDTPWEYCAIKMCAHSTMNDTDLPMQTTECIQGQGEGYRGTINTIWNGIPCQRWDSQYPHQHDITPENFKCKDLRENYCRNPDGAESPWCFTTDPNIRVGYCSQIPKCDVSSGQDCYRGNGKNYMGSLSKTRSGLTCSMWDKNMEDLHRHIFWEPDATKLNKNYCRNPDDDAHGPWCYTGNPLIPWDYCPISRCEGDTTPTIVNLDHPVISCAKTKQLRVVNGIPTRTNVGWMVSLKYRNKHICGGSLIKESWILTARQCFPSRNKDLKDYEAWLGIHDVHGRGDEKRKQVLNVTQLVYGPEGSDLVLLKLARPAILDDFVSTIDLPNYGCTIPEKTTCSVYGWGYTGLINSDGLLRVAHLYIMGNEKCSQYHQGKVTLNESEICAGAENIVSGPCEGDYGGPLVCEQHKMRMVLGVIVPGRGCAIPNRPGIFVRVAYYAKWIHKIILTYKAPQS

SEQ ID NO: 30 - protein, 711 aa, Oreochromis niloticus
MWIYKLVFGFVFVVSCSEGRRNALQDYQKTDSIQLMVHSDSSHLTKSRKLSLSKCAKACSRNKRLPFTCRAFVYDHKNRKCQWLSFDRNSPGVQSQQNMNYQLYEKKDYVRECIVGTGQSYRGRRSVTVSGILCQAWASPVPHEHKFMSKRFRKTDLRENYCRNPDNSAVGPWCFTTDPRPHFRHEECGIPQCSEVECINCIGEDYRGPMDHTESGKECQRWDLDDPHKHLYHPKRYPDKGLDDNYCRNPDGRQRPWCFTTDPNTPWEYCNITVCETPPKSNVVETTECYQGRGEGYRGTVDMTPTGLTCQRWDSQYPHNHTFLPEAYPCKDLRENFCRNPDGQEFPWCFTTDPRVRTMSCINIPQCGTQNRPVSDCYERFGENYQGQQSRTRSNLPCAPWRDHSNSGERGMPTAGLEGNYCRNPDKDKHGPWCYTNNSAIPWDYCNVKPCNASQNTIQLGEDASVSCFVHKTTRIVGGSPVSISEGSWMVSIQKGSMHWCGGSLIRDEWVLTDRECFSSCVPNLSEYRVWLGVSDIREGAPDWSKRQEVSIAHVICGPEGSSLALLRLSKPALPADNVHTIQLPVAGCTIPEGTICKMYGWGETKGTGYEDMLKAVELPIVRNNRCREMHRGNLHITTNKICAGGKRNEGVCERDYGGPLVCQDGYIRVIVGVSVHGRGCARANQPSIFINVPFYTQWIYKVFKYYPNTV

SEQ ID NO: 31 - protein, 1384 aa, Bos taurus
MKAPAVLAPGILVLLFTFVQKSNGECKEALVKSRMNVNMQYQLPNFTAETSIQNVVLHKHHIYLGAINYIYVLNDKDLQKVAEYKTGPVLEHPDCFPCQDCSHKANLSGGVWKDNINMALLVDTYYDDQLISCGSVHRGTCQRHVLPPNNTADIESEVHCMYSPQADEETNQCPDCVVSALGTKVLLSEKDRFINFFVGNTINSSYLPDYILHSISVRRLKETQDGFKFLTDQSYIDVLPELRDSYPIKYVHAFESNHFIYFLTVQRETLDAQTFHTRIIRFCSADSGLHSYMEMPLECILTEKRRKRSTKQEVFNILQAAYVSKPGAQLARQIGASLNDDILYGVFAQSKPDSSEPMNRSAVCAFPVKYVNEFFNKIVNKNNVRCLQHFYGPNHEHCFNRTLLRNSSGCEVRNDEYRTEFTTALPRVDLFTGQFNQVLLTSISTFIKGDLTIANLGTSEGRFMQVVVSRSGSLTPHVNFHLDSHPVSPEVIVEHPLNQNGYTLVVTGKKITKIPLNGLGCEHFQSCSQCLSAPSFVQCGWCHDKCVRLEECSSGTWTQETCLPTIYKVFPTSAPLEGGTTLTVCGWDFGFKRNNKFDLKKTRVLLGNESCTLTLTESTTNMLKCTVGPAMNEHFNMSIVISNSRGSVEYSAFSYVDPIITSISPNYGPKTGGTLLTLTGKHLNSGNSRHISIGGKTCTLKSVSHSILECYTPAQSAPTEFSVKLKIDLANREVNSFIYREDPIVYEIHPTKSFISGGSTITGVGKNLNSVSVLRMVINVHEAGRNFTVACQHRSNSEIICCTTPSIEQLNLQLPLKTKAFFMLDGIHSKYFDLIYVHNPVFKPFEKPVMISVGNENVLEIKGNDIDPEAVKGEVLKVGNKSCENIHSHSEAVLCTVPSDLLKLNSELNIEWKQAISSTVLGKVIVQPDQNFTGLIVGVVSISIILLLLLGLFLWLKKRKQIKDLGSELVRYDARVHTPHLDRLVSARSVSPTTEMVSNESVDYRATFPEDQFPNASQNGSCRQVQYPLTDLSPILTSGDSDISSPLLQNTIHIDLSALNPELVQAVQHVVIGPSSLIVHFNEVIGRGHFGCVYHGTLLDNDDKKIHCAVKSLNRITDIGEVSQFLTEGIIMKDFSHPNVLSLLGICLRSEGSPLVVLPYMKHGDLRNFIRNETHNPTVKDLIGFGLQVAKGMEYLASKKFVHRDLAARNCMLDEKFTVKVADFGLARDVYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAELRPSFSELVSRISVIFSTFIGEHYVHVNATYVNVKCVAPYPSLLSSQDNVSGEDDDDT

SEQ ID NO: 32 - protein, 1378 aa, Oreochromis niloticus
MNLCLLHAIMLLWAIRSSVQGQCDRSSEDTKLNLSVTYELPSFTAEFPIQNMVILDGVIYVGAVNRIYALAPNLRKLSEYHTGPLRANETCGQKLNGTSSSGRVDNHNIALVAENIYDKGLYSCGSADNGVCRRHVLDDGVSAKSVDEEVYCFADKKKLETGQPDDSDVVVSPLGSQVLNVESNVIQFFVGNSEIPGRRNISSSAKHPHTLSLRKMKTSQNGFTFFSNRSYMDLIPSLRGNYYLRYVYSFQSGPFTYFLTVQQVSKDSQAYHSRIVRMCSADSVIRRYVEMPLECISTDKRRRRRRSGTGVKVFNILQAAYVTKAGIDPEIQKHLKVEEDDDVLFAAFAKGKPNSKDPTPNSAVCVIALKQINSMFKTYMEKCNTIEPYHFTGLDKKSCYNVTSSDDCDPHEGIHEGKESTYRLQVTQFFQRLEYWHKDLTNTLVTSITVVPVHGRAVVYLGTADGRQIQVLLSRFASPHVNIHLDSRPVTTSVALLDADRSDGAILMATGNKITKVPLIGPGCGQLTTCISCLLSSRVTECGWCDGRCTRASECTSSIWTQDYCTPEITKVSPASGPIRGSTVVTICGKNFGFDKTENFKASMVTVEVAGASCKLSRQENSNRWTEMHCSPVFNGNFTPSGDVVKVTSGQNVTMFKGFNFVDPVIRDIFPTFGPKSGNTMLTITGANLDTGSKREVTIGKGICNVQSWSSKKVTCKTPPYAALSSQTVKLTVDSVERRAPMQFTYNQDPIINNIQPSRSFVSGGCTVSAHGLYLKSGLQPMMLVSIGENTETFHVSCVYGENQTSIQCTTPSLVKLGMQPPVITKVSFVLDGYSTKQMDMIYVEDPLFQDPKLTSKDNKSIVELKGDRMDREAMNCQVLTVSNHSCESLTLVGNTLECIVPTELQVATAKELQVEWRQADSIRHLGKVTLAQEQDYTGLIVGCVCVSLLLLLLGSLLMWKKNKHIDDQGTDRPETCRAPPPIYGGNGELLSPRLGALGGGIGMGLGMGMGIDGDLVSPLLMAPVHIDPSCLHPDLLTEVQHVVIARERLLLHLNQVIGRGHFGCVFHGTLLEPDGQKLHCAVKSLNRITDLEEVSQFLKEGIIMKDFSHPNVLSLLGICLPPEGSPLVVLPYMKHGDLRNFIRDEGHNPTVKDLMGFGLQVARGMEYLASKKFVHRDLAARNCMLDESYTVKVADFGLARDVYDKEYYSVHNKSGVKLPVKWMALESLQTHKFTTKSDVWSFGVLLWELMTRGAPPYSDVNSFDITVFLLQGRRLLQPEFCPDALYTVMIECWHPKPERRPSFSELVSRISAIFSSFSGEHYVLLNTTYVNIDKMSPYPSLLSSTASSSSSSSEPSTSTSLPSRSPLFCQVDRDCCT

SEQ ID NO: 33 - protein, 142 aa, Bos taurus
IDECRRGVHSCGENATCTNMEGNHTCTCAGDLSEPGQICPDSTLLSHLGKNGHNFLKKCFPEYTPNFEGYCLNGRVCIYFGIANLFSCHCPIGYPGKRGEYIDFDGWDPHSAGRGHQWNTSPVAVRALVLAFLLLLGLCRAH

SEQ ID NO: 34 - protein, 116 aa, Bos taurus
SRALEEKKVCQGTSNKLTQLGTFEDHFLSLQRMFNNCEVVLGNLEITYMQSSYNLSFFKTIQEVAGYALIALNTVEKIPLENLQIIRGNVLYENTHALAVLSNYGANKTGLKELPL

SEQ ID NO: 35 - protein, 1206 aa, Oreochromis niloticus
MTPSHRELQNNTMAARFLKWISLTSLLWLSFCGLAENKVCQGVTNRLNLLGTKEDHYRNMVKTYSNCTVVLENLEITYMEDHRDLSFLRSIKEVGGYVLIALNTVSRIPLENLRIIRGHSLYDGSFALSVISNYNKSTNKGTDQILLTSLTEILKGGVKIWTNQLCNVETIQWSDIVNVGNISIDPTPSKNKNCGKCDSSCFNGSCWSPGPQNCQTLTKLNCAQQCSRRCKGPSPSDCCNEHCAAGCTGPRAEDCLACRDFQDNGVCKDSCPGLHRYDPNLHQLVPNPHGKYSFGATCVKSCPRNYIISDYGACVRTCNGNTYEVEVGGIRKCAKCDGLCPKVCNGLGTGDLVHTLSINATNIGSFENCTKINGNIDIIHTSIYGDKFTKTPKMDPEQLNVFKTVKEITGYLWIQTWPDTMSSLSPFENLEIVRGRTKRGSRSLVVSGLHITHLGLRSLREISDGDVFVSKNSELCYTDAKHWQKLFKSPHQTVNIEGNANAATCALRNDTCDKKCTADGCWGPGPSMCISCRDYKRDGSCVDSCNILEGEPRETAVNKTCIKCHPECQPMNGTATCSAPGSANCTKCANFKDGLFCVSRCPQGVLGEDDALVWKYADEQKVCQLCHKNCTQGCIGPGLEGCHSKGPPGLSMVAAGVVGGLLAALIAGLSVFVLLRRRHIKRKRTMRRLLQERELVEPLTPSGEAPNQALLRILKEPEFKKIKVLGSGAFGTVYKGLWVPEGEDVKIPVAIKVLREATSPKANKEILDEAYVMASVEHPHVCRLLGICLTSTVQLITQLMPYGCLLDYVKENKDNIGSQYLLNWCVQIAKGMNYLEERHLVHRDLAARNVLVKTPQHVKITDFGLAKLLKADEKEYHADGGKVPIKWMALESILNRTYTHQSDVWSYGVTVWELMTFGTKPYDGIPASEIAGILEKGERLPQPPICTIDVYMIMVKCWMIDADSRPRFRELIAEFTKMARDPPRYLVIQGDDRMHLPSPSDTKFYRSLISGEDMEDAVDADEYLVPQRGFFSSPSTSRTPLIHSTSLNSSIGTCHSRTGLLNGFPSRDGSMALRYIPDPTDKFLDEAFQPAPGYINQTPISDVVNPIYQHPGPPRTLLPTISSDHTETEYLNYFKNGAAGPEYLNEILSPAVLPLTSNGTVHSIEKYLPQQSIDNPDYQQDFTPTFKTHTNGHIPAAENAEYLGPD

SEQ ID NO: 36 - protein, 416 aa, Bos taurus
MQALVLLLWTGALLGFGRCQNAGQEAGSLTPESTGAPVEEEDPFFKVPVNKLAAAVSNFGYDLYRVRSGESPTANVLLSPLSVATALSALSLGAEQRTESNIHRALYYDLISNPDIHGTYKDLLASVTAPQKNLKSASRIIFERKLRIKASFIPPLEKSYGTRPRILTGNSRVDLQEINNWVQAQMKGKVARSTREMPSEISIFLLGVAYFKGQWVTKFDSRKTSLEDFYLDEERTVKVPMMSDPQAVLRYGLDSDLNCKIAQLPLTGSTSIIFFLPQKVTQNLTLIEESLTSEFIHDIDRELKTVQAVLTIPKLKLSYEGELTKSVQELKLQSLFDAPDFSKITGKPIKLTQVEHRVGFEWNEDGAGTNSSPGVQPARLTFPLDYHLNQPFIFVLRDTDTGALLFIGKILDPRGT

SEQ ID NO: 37 - protein, 217 aa, Bos taurus
MMAAGPRTSLLLAFALLCLPWTQVVGAFPAMSLSGLFANAVLRAQHLHQLAADTFKEFERTYIPEGQRYSIQNTQVAFCFSETIPAPTGKNEAQQKSDLELLRISLLLIQSWLGPLQFLSRVFTNSLVFGTSDRVYEKLKDLEEGILALMRELEDGTPRAGQILKQTYDKFDTNMRSDDALLKNYGLLSCFRKDLHKTETYLRVMKCRRFGEASCAF

SEQ ID NO: 38 - protein, 204 aa, Oreochromis niloticus
MNSVVLLLSVVCLGVSSQQITDSQRLFSIAVNRVTHLHLLAQRLFSDFESSLQTEEQRQLNKIFLQDFCNSDYIISPIDKHETQRSSVLKLLSISYGLVESWEFPSRSLSGGSSLRNQISPRLSELKTGILLLIRANQDEAENYPDTDTLQHAPYGNYYQSLGGNESLRQTYELLACFKKDMHKVETYLTVAKCRLSPEANCTL

SEQ ID NO: 39 - protein, 634 aa, Bos taurus
MDLWQLLLTLAVAGSSDAFSGSEATPAFLVRASQSLQILYPVLETNSSGNPKFTKCRSPELETFSCHWTDGANHSLQSPGSVQMFYIRRDIQEWKECPDYVSAGENSCYFNSSYTSVWTPYCIKLTSNGGIVDHKCFSVEDIVQPDPPVGLNWTLLNISLTEIHADILVKWEPPPNTDVKMGWIILEYELHYKELNETQWKMMDPLMVTSVPMYSLRLDKEYEVRVRTRQRNTEKYGKFSEVLLITFPQMNPSACEEDFQFPWFLIIIFGILGLAVTLYLLIFSKQQRIKMLILPPVPVPKIKGIDPDLLKEGKLEEVNTILAIHDNYKHEFYNDDSWVEFIELDIDDPDEKTEGSDTDRLLSNDHEKSLNIFGAKDDDSGRTSCYEPDILEADFHVSDMCDGTSEVAQPQRLKGEADISCLDQKNQNNSPSNDAAPASQQPSVILVEENKPRPLLIGGTESTHQAVHTQLSNPSSLANIDFYAQVSDITPAGNVVLSPGQKNKTGNPQCDTHPEVVTPCQANFIVDNAYFCEVDAKKYIALAPHVEAESHVEPSFNQEDIYITTESLTTTAGRSGTAEHVPSSEIPVPDYTSIHIVQSPQGLVLNATALPLPDKEFLSSCGYVSTDQLNKIMP

SEQ ID NO: 40 - protein, 635 aa, Oreochromis niloticus
MALSPSSNLLILLILSSLDWLPSPGSTFLTDWDHTTSSALIEPHFTECISRDQETFHCWWSPGSFHNLSSPGALRVFYLKKEPPTSQWKECPEYIHSNRECFFDEAHTSIWITYCMQLRTQNNITYFNEDDCFTVENIVRPDPPVNLNWTLLNTSPSGLNYDVMINWEPPPTADVRLGWMRVEYELQYRERNTTNWEALDIQRQSHQTIYGLRLGKEYEVHIRCRMQAFIKFGEFSESVFIQVTEIPSTESTVHLTLVLVFGTVGILILIMLIVISQQNRLMIFLLPPVPAPKIKGIDSELLKKGKLDELNFMLSGRGMDGLPIYAPDFYQDEPWVELMEVDETEDVDNGEKKDNRGSDTQKLLGQSQPVSQHININCSNSVSGPDAESSQATCYNTDLPEEETLMLMATLLPGQPDEEETSLDTVERSSASETGERQLIQTQTRGPQTWVNTDFYAQVTNVMPTGGVVLSPGQQLRIQESISAAEKETKKKRKESEDSEESEERKQKEPQFQLLVVDPEGSAYSTESSIQQISTPPPSSPMPGEGYHIIHPQPVEPRPAATMELNQSPYIIPDSPQFFAPVADYTVVQELDSHHSLLLNPPCHQTPPPCLPQHPLKAPMPVGYITPDLLGNLSQ

SEQ ID NO: 41 - protein, 208 aa, Bos taurus
MNSRFTSAFTPFAVSLGLLLVMTSAFPTPGPLGEDFKNDTTPGRLLLTTPEKTEALIKRMVDKISAMRKEICEKNDECESSKETLAENKLNLPKMEEKDGCFQSGFNQAICLIRTTAGLLEYQIYLDYLQNEYEGNQENVRDLRKNIRTLIQILKQKIADLITTPATNTDLLEKMQSSNEWVKNAKIILILRNLENFLQFSLRAIRMK

SEQ ID NO: 42 - protein, 220 aa, Oreochromis niloticus
MPSIFNLHFSSAVMLAALLLCASGAPIEDGSVAGDFSGEETEEAEMSTVKPFNIWRSLFDSAQEYEKAFEHHFQTLENRDQALDSHTPASIPKHCNITKFRKDACLQTLAKGLLIYSVLLKHVEKEYRSSLNFSDAPSNIGTLIDLAGMVKGKMKNRNQVTPLTSSEEEQLLKEVNSPDPYHRKLHAYSILRALKAFLSEGKRAVCRMEKRVNQSADTSC

SEQ ID NO: 43 - protein, 464 aa, Bos taurus
MLAVRCALLTALLAASGTALVLKGCPALEVASDVLTSVPGANVTLTCPGGGPEDNATVHWVLRDQMTGSHHGRPAGVGRRLLLQSVQLSDSGNYSCYREDHPAGSVRLVVDVPPEKPQISCIQKSPLSRVNCEWSPRSPPSLTTKAVLLVKKFPKQVFQEPCQYSPESQRFSCQLAVPEGDSSFYVLSLCVANSAGNKSSNPLGFDGYKLLQPDPPVNITVSAVDRNPRWLSVTWQDPPSWNSYYYRLQFELRYRTERSETFTTLMLKDPQYHCIINDAWSGMRHVVQLRAQEEFGNGLWSEWSPEVTGTPWTESRSSTTTKLPTSTQAPTTTEDNENIFSKDSAKTTSLPVQDSTSVPLPAFLVAGGSLAFGTLLCIGIILRFKKTGKLQALKEGKTSAHPSYSLGQLVPERPKPTPALVPLISPPVSPSSLESDNTSRNSRPDAKGPGSPYDVSNRDYFFPR

SEQ ID NO: 44 - protein, 892 aa, Oreochromis niloticus
MVSAAVLHLCVLAWVASVLAADDYHLVIAPQSPVVQIGSNFTATCMIINTTEVTADDLYWKCSEIIPKEHYIKINTSALNVTVTIRSEKSEWLLCKCKTVSQYVALNEGKFIHGILLRKGYRPEKPKNLSCIAVHESEKISSNIRCQWEAVGNQTEAVPTNYTLFVRQTGSDKVYTASILGPVNSTTVDMNFFPHHMELEIWVVAQNELGKEESEHLIKLANCFVKTKPPSVTVISEAVFPTSLVMNWTSSIDKRYLETIYEIRYCPLGSQMWIYVPLEDTSRYIQSFRLQNLTPDTVYVTQVRCKYNNKNCGYWSDWSRNVTERTPEDLPTSKPDVWIITSAEGINKRQIEFICKDPEVSNGRIRRFNITIEKQKYRIRNGTWEIIPVNRSLTDGSSSKKQFTPLKTIRLDDEESVTVYVTAINSVGASPQAKLIIYRKAREPPAVEKMEVQFLDGQLLISWKSPNRTVSEYVVEWVGDGERNWQRENGKANCSTIKGDLKRFVHYRISVYPIYGKVVGKPVHEQAYAEQGVPSQAPTVNLSGDPGCYTAKLEWDEIPLIKRRGFITNYTIYYRSGTETHGIIVPPNTTSYTLTSLSRNTKYDIWIKASTIRGSVNGSNHSFTTLKYAPGEIEGIVVGVSLGFLFVVLMTMLLCFCKKDVIKDNFWPQIPNPGDSTIGNWSPDYPLKAEAPRENCLSGISVLNVDVCDAKCVFEEDKASLPLKKDKYLSEEHSSGIGGSSCMSSPRQSVSDSDEGGDMADTTASTIQYSSVVASSGYKGQNPSSLPQQSIFSRSESTQPLLDCEENHDTMVQEGSRQCFLRQPCFNHNAGNENATDSNDFNPLEMEQQEVLDFCPLEEDTEHTAPTDCQSDDWIPATVSSYMPQLGGYRPQ

SEQ ID NO: 45 - protein, 300 aa, Bos taurus
MVPAPRAQPARGPAGWRARQGAPLPSSSSSSLLNSRRCSSRSALSGIHPPSFLSFSFFPLSFPTASPAAPWEGRRPPEQLANSGPGREQVPPPSARAGKLWSVLGVVPLLLVLHWKHGAGSPLPITPVNATCATRHPCPSNLMNQIRNQLGQLNSSANSLFILYYTAQGEPFPNNLDKLCSPNVTDFPPFHANGTEKARLVELYRIIAYLGASLGNITRDQKVLNPYAHGLHSKLSTTADVLRGLLSNVLCRLCSKYHVSHVDVTYGPDTSGKDVFQKKKLGCQLLGKYKQVIAVLAQAF

SEQ ID NO: 46 - protein, 991 aa, Oreochromis niloticus
MAHLSLTPTLKLISSGFVWLLWIVLWFSTSHTHADTGLTVPQQVTLSPNWGTQELSISWLGGGATTFDLIILRTEFNETVFYETVSPTVNKVSGLHQWTWTSVNPLECTSLSVKIRSRAGQYTSEWSQTETLQGHDHPNSEQRVKAFPQDKVLPVGANITFCCIMDERMFFDKIVYDGNATIVTRLSRRTYSTTVSNLVASRPSGSNMLCLPEDKGKLYGAVVFVGYPPLPTDFKCETYDLRSAVCQWGKGRDTHLYGIQRGTRYFVNNRDCTEVNQDVGQKELNCTLDTWEGNWTLVAKNLLGQYSLTDTAELSHRVCPVAPVQLRVVADAWNASLSWHWKYDSYSSLALDCQVEVTATEDEKLIKRVFSGVGLRSVFLSDLYPDERYSVKVRCGVQQNFWKWGKWSELFSFKTKTYVPDTPDAWIWLNTDNTGTVVWKPLTRRQSHGKLTQYEVFLWSPEENRALAPTVTLPPNTWSMPVNLTEIAFFSNNSSIVANVTARNDAGSSSPARVHLTSVEPLAASRIVYTDQGFPLFWENNANATCGYVVEWLDAFCSQGCPVEWIKLPAGTTNVSLESDTFQPGVRYNLSLYSCSSDSRELLKRWQGYAQELVPSVALTLSASQQNADILLTWNEIPLVNRRGFLLGYNIYNSTGYQFDLLANLPDPETMSYTAKDFTKGTYKFTVKAYTAAGENTGTTATVTIEPYADWLILEILTSLGLVTLLLAIVTFTCYKKRKWVKKAFYPDIPEPKLPSDWPRTQGPLDVKPSPHSMVCVVETPEYDCIKEMLVAIPEEDEDDEGQGIGDESVDTDEPMSLRYYNQMVDERPIRPRFPDSSASSLSSMGSAHTDVTYTGIQTSGSSLVLQLDPQGSTECYQPQLDQSVSYGGGGYRPQMQPRAPSDDMGLPSPESFLEPQAACSGGYKPQGSWHLDSPVDAAEMGSLAPSLGSPTSVASTQFLLPDGEENRGEKRQHSSSAASWLTNLLSSTKP

SEQ ID NO: 47 - protein, 234 aa, Bos taurus
MSTKSMIRDVELAEEVLSEKAGGPQGSRSCLCLSLFSFLLVAGATTLFCLLHFGVIGPQREEQSPGGPSINSPLVQTLRSSSQASSNKPVAHVVADINSPGQLRWWDSYANALMANGVKLEDNQLVVPADGLYLIYSQVLFRGQGCPSTPLFLTHTISRIAVSYQTKVNILSAIKSPCHRETPEWAEAKPWYEPIYQGGVFQLEKGDRLSAEINLPDYLDYAESGQVYFGIIAL

SEQ ID NO: 48 - protein, 247 aa, Oreochromis niloticus
MVAYTTTPVDVEAGPEAKTVVLVEKKSPAEWIWKVCAVLVVVALCLAGVLLFAWYWNTRPERMTQLGQPEALKAKNTGDKTEPHSTLKRISSKAKAAIHLEGSDSKGHLEWRNGQGQAFAQGGFKLEANKIIIPHTGLYFVYSQASFRVICGNTDENEDEEKSLTILSHRIWRYSESMGSSSTLMSALRSACQDTIQDSFSDHGWYNAIYLGAVFQLNEGDTLWTETNQLSELETDEGRTFFGVFAL

SEQ ID NO: 49 - protein, 190 aa, Bos taurus
MNFLLSWVHWSLALLLYLHHAKWSQAAPMAEGGQKPHEVVKFMDVYQRSFCRPIETLVDIFQEYPDEIEFIFKPSCVPLMRCGGCCNDESLECVPTEEFNITMQIMRIKPHQSQHIGEMSFLQHNKCECRPKKDKARQENPCGPCSERRKHLFVQDPQTCKCSCKNTDSRCKARQLELNERTCRCDKPRR

SEQ ID NO: 50 - protein, 153 aa, Bos taurus
APESIFDKIYSTKSDVWSYGVLLWEIFSLGGSPYPGVQMDEDFCSRLKDGMRMRAPEYATPEIYQIMLDCWHKDPKERPRFVELVEKLGDLLQANVQQDGKDYIPLNAILTGNTAFTYSTPAFSEDFFQEDISAPKFNSGSSDNVRYVNAFNF

SEQ ID NO: 51 - protein, 155 aa, Bos taurus
MAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYSSWYVALKRTGQYKLGPKTGPGQKAILFLPMSAKS

SEQ ID NO: 52 - protein, 155 aa, Oreochromis niloticus
MATGGITTLPATPEDGGSSGFPPGNFKDPKRLYCKNGGFFLRIKSDGGVDGIREKNDPHIKLQLQATSVGEVVIKGICANRYLAMNRDGRLFGARRATDECYFLERLESNNYNTYRSRKYPNMYVALKRTGQYKSGSKTGPGQKAILFLPMSAKC

SEQ ID NO: 53 - protein, 83 aa, Artificial Sequence
  Synthetic- IGF-1_LR3
MFPAMPLSSLFVNGPRTLCGAELVDALQFVCGDRGFYFNKPTGYGSSSRRAPQTGIVDECCFRSCDLRRLEMYCAPLKPAKSA

SEQ ID NO: 54 - protein, 499 aa, Bos taurus; identical to SEQ ID NO: 14

SEQ ID NO: 55 - protein, 221 aa, Oreochromis mossambicus
HHRVPNEEDPSMDHPFITVGTTLKDLIYDMTTSGSGSGLPLLVQRTIARTIILQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLSLSTASGLAHLHMEIVGTQGKPAIAHRDLKSKNILVKKNGTCCIADLGLAVRHDSATDTIDIAPNHRVGT

SEQ ID NO: 56 - protein, 118 aa, Bos taurus
  misc_feature (89)..(89): Xaa can be any naturally occurring amino acid
ESAGECCSCPKTDSQILKELEESSFRKTFEDYLHNVVFIPRPSRKRRALGDVGNVTAAVPTALGLPNTSSTSTPMSSEEHRPFEKVVNXESLVISGLRHFTGYRIELQACNQDSPEER

SEQ ID NO: 57 - protein, 146 aa, Oreochromis niloticus
FGMVYEGIAKDIVKGEGETRVAVKTVNESASLRERIEFLNEASVMKAFSCHHVVRLLGVVSKGQPTLVVMELMTHGDLKSFLRSLRPDAENNPGRPPPTLKEMIQMAAEIADGMAYLNAKKFVHRDLAARNCMVAHDLTVKMGDLE

SEQ ID NO: 58 - protein, 486 aa, Bos taurus
MFPKETTWNISFAGCGFLGVYHIGVASCLREHAPFLVANATHIYGASAGALTATALVTGACLGEAGANIIEVSKEARKRFLGPLHPSFNMVKTIRGCLLKILPADCYECASGRLGISLTRVSDGENVIITHFNSKEELIQANVCSTFIPVYCGLIPPSLQGVRYVDGGISDNLPLYELKNTITVSPFSGESDICPQDSSTNIHELRVTNTSIQFNLRNLYRLSKALFPPEPLVLREMCKQGYRDGLRFLRRNGLLNRPNPLLALPPSQPPAPEDADAQEGAVAMERTGGKDHLPPPREDHILEHLPSRLNEALLEACMEPTDLLTTLSNMLPVRLAMAMMVPYTLPLESAVSFTIRLLEWLPDVPEDIRWMKEQTGSICQYLMIRAKRKLGNHLPSRLSGQVVLRRARSLPSVPLSCAAYSEVLPSWMRNSLSLGDVLAKWEECQRQLLLGLFCTNVAFPPDALRMRVPAGPAPEPPQHPPSSPPC

SEQ ID NO: 59 - protein, 24 aa, Artificial Sequence
  Synthetic- Mechanogrowth factor
YQPPSTNKNTKSQRRKGSTFQQRK

SEQ ID NO: 60 - protein, 390 aa, Bos taurus; identical to SEQ ID NO: 12

SEQ ID NO: 61 - protein, 381 aa, Oreochromis niloticus
MKLWFLILMVVYMVGSVHNLSTCNTVDLEMVKKKRIEAIRSQILTKLRLQKAPDEAGEKEEIPANLLSLYNSTSEILLEKQDEEQKIIPREQEEEEYFAKVLNRFNMTTKNDTNINYKPKVISMSFNISEIRSNVGDGLLLTRAELRMLIKEPMILNEERVELYHSQGTSTHYLASRFVTNTLKDKWLSFDVTEPLQTWLQGNENEQKFELRRYCECGNNDDTLSFSISGTMSRRGDTKDLQKLNQQLPYIFTMSIPKTNHSKSLRTKRSTDTTDSCGTQSHNCCLKKLYIDFRKDLGWKWIHKPTGYYANYCMGSCTYIWDAENKYSQILALYKHHNPGASAQPCCAPQTLEPLPIIYYVGRQHKVEQLSNMIVTSCKCS

SEQ ID NO: 62 - protein, 414 aa, Bos taurus
MHYCVLSAFLLLHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPSFLPSENAIPPTFYRPYFRIVRFDVSSMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKSPHLLLMLLPSYRLESQQSNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 63 - protein, 407 aa, Oreochromis niloticus
MNVYLVCLFLTLDLATVAFSLSTCSTLDMDQFKKKRIEAIRGQILSKLKLAAPPKDFPEPEEVSRDIVSIYNSTRDLLQEKANERAATCERQRSEEEYYAKEVHKIDMQPVHSPENVISPTPFNPYFRWLTFDVSSMEKNASNLVKAELRIFRLQNPGARVSEQRIELYQILGYKGLTSPTQRYIDSKVIRTQTEGEWLSFDVTEAVSEWLNHRDRNSGFKISLHCPCCTFVPSNNYIIPNNSEELEARFAGIDDNLIHGRDLKVFRKWQHSVRGPHLLLMLLPSYRLESQSQHKNHRSKRALDTAYCSKNVQDNCCLRSLYIDFKRDLGWRWIHEPKGYEANFCAGACPYLWSADTQHSKVLGLYNTINPEASASPCCVSQDLEPLTILYYIGKTPKIEQLSNMKN

SEQ ID NO: 64 - protein, 199 aa, Artificial Sequence
  Synthetic- puromycin resistance
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 65 - protein, 238 aa, Artificial Sequence
  Synthetic- GFP
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 66 - protein, 21 aa, Artificial Sequence
  Synthetic- T2A peptide
GSGEGRGSLLTCGDVEENPGP

SEQ ID NO: 67 - protein, 22 aa, Artificial Sequence
  Synthetic- P2A peptide
GSGATNFSLLKQAGDVEENPGP

SEQ ID NO: 68 - protein, 142 aa, Bos taurus; identical to SEQ ID NO: 33

SEQ ID NO: 69 - protein, 1200 aa, Bos taurus
MRLSGAGRAALLVLLAAHFQTSLALEEKKVCQGTSNKLTQLGTFEDHFLSLQRMFNNCEVVLGNLEITYMQSSYNLSFLKTIQEVAGYVLIALNTVEKIPLENLQIIRGNVLYENTHALAVLSNYGANKTGLRELPLRNLQEILQGAVRFSNNPVLCNVETIQWRDIVNPDFLSNMTGDFQNQQGNCPKCDPACLNRSCWGAGEENCQKLTKIICAQQCSGRCRGRSPSDCCHNQCAAGCTGPRESDCLVCRRFRDEATCKDTCPPLMLYDPTTYEMKVNPLGKYSFGATCVKKCPRNYVVTDHGSCVRACSSDSQEVEEDGVRKCKKCDGPCGKVCNGIGIGEFKDTLSINATNIKHFRNCTSISGDLHILPVAFRGDSFTRTAPLDPKELDILRTVKEITGFLLIQAWPENRTDLHAFENLEIIRGRTKQHGQFSLAVVGLDITSLGLRSLKEISDGDVIISGNRNLCYADTIRWKKLFGTSTQKTKILNNRSEKQCKAAGHICHPLCSSEGCWGPGPKYCMSCQNFSRGKECVGKCNILEGEPREFVENSECVQCHPECLPQAMNVTCTGRGPGNCVKCAHYIDGPHCVKTCPAGVAGENGTLIWKFADANHVCLLCHPNCTYGCEGPGLEGCPQKGPKIPSIATGIVGGLLLVVVLALSVGLFMRRRHIVRKRTLRRLLQERELVEPLTPSGEAPNQALLRILKETEFKKVKVLGSGAFGTVYKGLWIPEGEKVKIPVAIKELREATSPKANKEILDEAYVMASVDNPHVCRLLGICLTSTVQLITQLMPFGCLLDYVREHKDNVGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISTVLEKGERLPQPPICTIDVYMIMVKCWMIDADSRPKFRELILEFSKMARDPQRYLVIQGDERMHLPSPTDSNFYRALMDEEDMEDVVDADEYLVPQQGFFHSPTTSRTPLLSSLSTSSNTPTVTCVDRNGSYPLKEDSFLQRYSSDPTGALIEDSMDDTFLPVPEYVNQSVPKRPAGSVQNPVYHNQPLYPAPGRDPQYQNSLSNAVDNPEYLNTTHPACINGVLDGPALWAQKGSHQFSLDNPDYQQAFFPKEAKSNGIFKGPAAENAEYLRAAP

SEQ ID NO: 70 - protein, 154 aa, Bos taurus
MGKISSLPTQLFKCCFCDFLKQVKMPITSSSHLFYLALCLLAFTSSATAGPETLCGAELVDALQFVCGDRGFYFNKPTGYGSSSRRAPQTGIVDECCFRSCDLRRLEMYCAPLKPAKSARSVRAQRHTDMPKAQKEVHLKNTSRGSAGNKNYRM

SEQ ID NO: 71 - protein, 408 aa, Homo sapiens
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEVISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 72 - protein, 409 aa, Bos taurus
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEEQIQGIGLEYPERPASRANTVRSFHHEEHLENIPGTSENSAFRFLFNLSSIPENEVISSAELRLFREQVDQGPDWDQGFHRINIYEVMKPPAEVVPGHLITRLLDTRLVHHNVTRWETFDVSPAVLRWTREKQPNYGLAIEVTHLHQTRTHQGQHVRISRSLPQGSGDWAQLRPLLVTFGHDGRGHALTRRRRAKRSPKHHPQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 73 - protein, 505 aa, Bos taurus
MGETLGDALIDPESEPFAVTVSARTSQEITMVDTEMPFWPTNFGISSVDLSMMDDHSHAFDIKPFTTVDFSSISTPHYEDIPFPRADPMVADYKYDLKLQEYQSAIKVEPVSPPYYSEKTQLYSKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHISPLQEPSKEVAIRIFQGCQFRSVEAVQEITEYAKNIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 74 - protein, 353 aa, Bos taurus
MESADFYEAEPRPPMSSHLQSPPHAPSSAAFGFPRGAGPSQPPAPPAAPEPLGGICEHETSIDISAYIDPAAFNDEFLADLFQHSRQQEKAKAAAAPAGGGNDFDYPGAPVGPGGAVMPGGTHGPPPGYGCAAAGYLDSRLEPLYERVGAPALRPLVIKQEPREEDEAKQLALAGLFPYQPPPPPPPPNSHPPPAHLAAPHLQFQIAHCGQTTMHLQPGHPTPPPTPVPSPHPAPALGAAGLPGPGGALKGLVATHPDLRAGGGGGGKAKKSVDKNSNEYRVRRERNNIAVRKSRDKAKQRNVETQQKVLELTSDNDRLRKRVEQLSRELDTLRGIFRQLPESSLVKAMGNCA

SEQ ID NO: 75 - protein, 500 aa, Homo sapiens
MGETLGDSPIDPESDSFTDTLSANISQEMTMVDTEMPFWPTNFGISSVDLSVMEDHSHSFDIKPFTTVDFSSISTPHYEDIPFTRTDPVVADYKYDLKLQEYQSAIKVEPASPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEI

SEQ ID NO: 76 - protein, 498 aa, Gallus gallus
MVDTEMPFWPVNFGISPVDLSAMDDHMHSFDIKPFTTVDFSSISSPHYEDIPLGRADQTSIDYKYDIKLQDCQISVEKKSSWFMSIAKQETKHSHYGAIKMEPPSPPYFSEKVQLYNKPHEESSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLRMGEDQIKCKHASPLQEQNKEVAIRIFQRCQFRSVEAVQEITEFAKNIPGFVNLDLNDQVTLLKYGVHEIIYTLLASLMNKDGVLISDGQGFMTREFLKSLRKPFCDFMEPKFEFAVKFNALELDDSDLAIFIAVIILSGDRPGLLNVKPIEDIQDNLLQALELQLKLNHPESSQLFAKLLQKMTDLRQIVTEHVQLLQIIKKTETDMSLHPLLQEIYKDLY

SEQ ID NO: 77 - protein, 505 aa, Mus musculus
MGETLGDSPVDPEHGAFADALPMSTSQEITMVDTEMPFWPTNFGISSVDLSVMEDHSHSFDIKPFTTVDFSSISAPHYEDIPFTRADPMVADYKYDLKLQEYQSAIKVEPASPPYYSEKTQLYNRPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKNIPGFINLDLNDQVTLLKYGVHEIIYTMLASLMNKDGVLISEGQGFMTREFLKNLRKPFGDFMEPKFEFAVKFNALELDDSDLAIFIAVIILSGDRPGLLNVKPIEDIQDNLLQALELQLKLNHPESSQLFAKVLQKMTDLRQIVTEHVQLLHVIKKTETDMSLHPLLQEIYKDLY

SEQ ID NO: 78 - protein, 505 aa, Capra hircus
MGETLGDPLIDPESEPFADTVSASTSQEITMVDTEMPFWPTNFGISSVDLSMMDDHSHAFDIKPFTTVDFSSISAPHYEDIPFPRADPMVADYKYDLKLQEYQSAIKVEPVSPPYYSEKTQLYSKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHISPLQEPSKEVAIRIFQGCQFRSVEAVQEITEYAKNIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 79 - protein, 504 aa, Sus scrofa
MGETLGDSLIDPESDAFDTLSANISQEVTMVDTEMPFWPTNFGISSVDLSVMDDHSHSFDIKPFTTVDFSSISTPHYEDIPFPRADPMVADYKYDLKLQDYQSAIKVEPVSPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKNIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 80 - protein, 359 aa, Mus musculus
MESADFYEVEPRPPMSSHLQSPPHAPSNAAFGFPRGAGPAPPPAPPAAPEPLGGICEHETSIDISAYIDPAAFNDEFLADLFQHSRQQEKAKAAAGPAGGGGDFDYPGAPAGPGGAVMSAGAHGPPPGYGCAAAGYLDGRLEPLYERVGAPALRPLVIKQEPREEDEAKQLALAGLFPYQPPPPPPPPHPHASPAHLAAPHLQFQIAHCGQTTMHLQPGHPTPPPTPVPSPHAAPALGAAGLPGPGSALKGLAGAHPDLRTGGGGGGSGAGAGKAKKSVDKNSNEYRVRRERNNIAVRKSRDKAKQRNVETQQKVLELTSDNDRLRKRVEQLSRELDTLRGIFRQLPESSLVKAMGNCA

SEQ ID NO: 81 - protein, 324 aa, Gallus gallus
MEQANFYEVDSRPPMSSGQHHQLQTPLPGSAYGYREAPSAAAPAAGGAELGDICENEHSIDISAYIDPAAFNDEFLADLFQHSKQQEKHKAVLAGDFDFHGMHGAGAAASAPGHHPQHQQQPLFGCAAGYMDGKLDPLYERIAAPGLRPLVIKQEPREEEEVKAAALAALYPHPQQHPSHLQYQIAHCAQTTVHLQPGHPTPPPTPVPSPHHPHHPHPPGALPAAPGALKMMPADHRGKSKKTVDKNSNEYRVRRERNNIAVRKSRDKAKQRNVETQQKVLELTTDNERLRKRVEQLSRELETLRGIFRQLPESSLVKAMGSCA

SEQ ID NO: 82 - protein, 353 aa, Capra hircus
MESADFYEAEPRPPMSSHLQSPAHAPSSAAFGFPRGAGPSQPPAPPAAPEPLGGICEHETSIDISAYIDPAAFNDEFLADLFQHSRQQEKAKAAAAPAGGGNDFDYPGAPVGPGGAVMPGGTHGPPPGYGCAAAGYLDSRLEPLYERVGAPALRPLVIKQEPREEDEAKQLALAGLFPYQPPPPPPPPHSHPPPAHLAAPHLQFQIAHCGQTTMHLQPGHPTPPPTPVPSPHPAPALGAAGLPGPGGALKGLAAAHPDLRAGGGGGGKAKKSVDKNSNEYRVRRERNNIAVRKSRDKAKQRNVETQQKVLELTSDNDRLRKRVEQLSRELDTLRGIFRQLPESSLVKAMGNCA

SEQ ID NO: 83 - protein, 1134 aa, Mus musculus
MDELAFGEAALEQTLAEMCELDTAVLNDIEDMLQLINNQDSDFPGLFDAPYAGGETGDTGPSSPGANSPESFSSASLASSLEAFLGGPKVTPAPLSPPPSAPAALKMYPSVSPFSPGPGIKEEPVPLTILQPAAPQPSPGTLLPPSFPAPPVQLSPAPVLGYSSLPSGFSGTLPGNTQQPPSSLPLAPAPGVLPTPALHTQVQSLASQQPLPASAAPRTNTVTSQVQQVPVVLQPHFIKADSLLLTAVKTDAGATVKTAGISTLAPGTAVQAGPLQTLVSGGTILATVPLVVDTDKLPIHRLAAGSKALGSAQSRGEKRTAHNAIEKRYRSSINDKIVELKDLVVGTEAKLNKSAVLRKAIDYIRFLQHSNQKLKQENLTLRSAHKSKSLKDLVSACGSGGGTDVSMEGMKPEVVETLTPPPSDAGSPSQSSPLSFGSRASSSGGSDSEPDSPAFEDSQVKAQRLPSHSRGMLDRSRLALCVLAFLCLTCNPLASLFGWGILTPSDATGTHRSSGRSMLEAESRDGSNWTQWLLPPLVWLANGLLVLACLALLFVYGEPVTRPHSGPAVHFWRHRKQADLDLARGDFPQAAQQLWLALQALGRPLPTSNLDLACSLLWNLIRHLLQRLWVGRWLAGQAGGLLRDRGLRKDARASARDAAVVYHKLHQLHAMGKYTGGHLAASNLALSALNLAECAGDAISMATLAEIYVAAALRVKTSLPRALHFLTRFFLSSARQACLAQSGSVPLAMQWLCHPVGHRFFVDGDWAVHGAPPESLYSVAGNPVDPLAQVTRLFREHLLERALNCIAQPSPGAADGDREFSDALGYLQLLNSCSDAAGAPACSFSVSSSMAATTGPDPVAKWWASLTAVVIHWLRRDEEAAERLYPLVEHIPQVLQDTERPLPRAALYSFKAARALLDHRKVESSPASLAICEKASGYLRDSLASTPTGSSIDKAMQLLLCDLLLVARTSLWQRQQSPASVQVAHGTSNGPQASALELRGFQHDLSSLRRLAQSFRPAMRRVFLHEATARLMAGASPARTHQLLDRSLRRRAGSSGKGGTTAELEPRPTWREHTEALLLASCYLPPAFLSAPGQRMSMLAEAARTVEKLGDHRLLLDCQQMLLRLGGGTTVTSS

SEQ ID NO: 84 - protein, 232 aa, Gallus gallus
  misc_feature (226)..(226): Xaa can be any naturally occurring amino acid
KTDAGGAKTSSITSLATSSTAAPLQVPALVSGGTILATVPLVVDAEKLPINRLAPSGKAALLQSKGEKRTAHNAIEKRYRSSINDKIVELKDLVVGTEAKLNKSAILRKAIEYIRFLQQSNQKLKQENLSLKMAVQKSKSLKDLVASCGAGPKAEASMEVAKAEVMEMPTPPPSDVGSPSRGSPLSLSGGSSNSSSDSEPDSPLCNHGKVKQENPPPSPGSQGMLXRSRMAL

SEQ ID NO: 85 - protein, 132 aa, Bos taurus
MCDAFVGTWKLVSSENFDDYMKEVGVGFATRKVAGMAKPTLIISLNGGVVTIKSESTFKNTEISFKLGQEFDEITPDDRKVKSIVNLDEGALVQVQNWDGKSTTIKRKLMDDKMVLECVMNGVTATRVYERA

SEQ ID NO: 86 - protein, 1292 aa, Bos taurus
MSRRKQAKPRSVKVEEGEASDFSLAWDSSVTASGGLEGEPECDRKTSRVLEDRNSVTSQEERNEDDEDVEDESIYTCDHCQQDFESLAELTDHRAHRCPGDGDDDPQLSWVASSPSSKDVASPTQMIGDGCDLGLGEEEGGTGLPYPCQFCDKSFIRLSYLKRHEQIHSDKLPFKCTYCSRLFKHKRSRDRHIKLHTGDKKYHCHECEAAFSRSDHLKIHLKTHSSSKPFKCTVCKRGFSSTSSLQSHMQAHKKNKEHLAKSEKEAKKDDFMCDYCEDTFSQTEELEKHVLTRHPQLSEKADLQCIHCPEVFVDENALLAHIHQAHANQKHKCPMCPEQFSSVEGVYCHLDSHRQPDSSNHSVSPDPVLGSVASMSSATPDSSASVERGSTPDSTLKPLRGQKKMRDDGQGWSKVVYSCPYCSKRDFNSLAVLEIHLKTIHADKPQQSHTCQICLDSMPTLYNLNEHVRKLHKNHAYPVMQFGSISAFHCNYCPEMFADINSLQEHIRVSHCGPNANPPDGNNAFFCNQCSMGFLTESSLTEHIQQAHCSVGSAKLESPVVQPTQSFMEVYSCPYCTNSPIFGSILKLTKHIKENHKNIPLAHSKKSKAEQSPVSSDVEVSSPKRQRLSASANSISNGEYPCNQCDLKFSNFESFQTHLKLHLELLLRKQACPQCKEDFDSQESLLQHLTVHYMTTSTHYVCESCDKQFSSVDDLQKHLLDMHTFVLYHCTLCQEVFDSKVSIQVHLAVKHSNEKKMFRCTACNWDFRKEADLQVHVKHSHLGNPARAHKCIFCGETFSTEVELQCHITTHSKKYNCKFCSKAFHAIILLEKHLREKHCVFDAAAENGTANGVPPAAAKKAEPADLPGVLLKNPEAPNSHEASEDDVDASEPMYGCDICGAAYTMEVLLQNHRLRDHNIRPGEDDGSRKKAEFIKGSHKCNVCSRTFFSENGLREHLQTHRGPAKHYMCPICGERFPSLLTLTEHKVTHSKSLDTGTCRICKMPLQSEEEFIEHCQMHPDLRNSLTGFRCVVCMQTVTSTLELKIHGTFHMQKLAGSSAASSPNGQGLQKLYKCALCLKEFRSKQDLVKLDVNGLPYGLCAGCMARSANGQVGGLAPPEPADRPCAGLRCPECSVKFESAEDLESHMQVDHRDLTPETSGPRKGAQTSPVPRKKTYQCIKCQMTFENEREIQIHVANHMIEEGINHECKLCNQMFDSPAKLLCHLIEHSFEGMGGTFKCPVCFTVFVQANKLQQHIFAVHGQEDKIYDCSQCPQKFFFQTELQNHTMSQHAQ

SEQ ID NO: 87 - protein, 673 aa, Bos taurus
MDLTKMGMIQLQNPSHPTGLLCKANQMRLAGTLCDVVIMVDSQEFHAHRTVLACTSKMFEILFHRNSQHYTLDFLSPKTFQQILEYAYTATLQAKAEDLDDLLYAAEILEIEYLEEQCLKILETIQASDDNDTEATMAEGGAEEEEDRKARYLKNIFISKHSSEESGYASVAAQSLPGPMVDQSPSVSTSFGLSAMSPTKAAVDSLMTIGQSLLQGALQPPAGPEEPTLAGGGRHPAVAEVKPEMMQVDEVPGQESPGTVESSISAGMGDKVEERGKEGPGTPTRSSVITSARELHYGREESTEQLPPPVEAGQGPPGRLEPPAHPAEKHLGLYSVLPNHKADAVLSMPSSVTSGLHVQPALAVSMDFSTYGGLLPQGFIQRELFSKLGELAVGMKAESRSVGEQCSVCGVELPDNEAVEQHRKLHSGMKTYGCELCGKRFLDSLRLRMHLLAHSAGAKAFVCDQCGAQFSKEEALETHRQTHTGTDMAVFCLLCGKRFQAQSALQQHMEVHAGVRSYICSECNRTFPSHTALKRHLRSHTGDHPYECEFCGSCFRDESTLKSHKRIHTGEKPYECNGCGKKFSLKHQLETHYRVHTGEKPFECKLCHQRSRDYSAMIKHLRTHNGASPYQCTICSEYCPSLSSMQKHMKGHKPEEIPPDWRIEKTYLYLCYV

SEQ ID NO: 88 - protein, 516 aa, Bos taurus
MAVNKGPTLLDGDLPEQENVLQRVLQLPVVSGTCECFQKTYASTKEAHPLVASVCNAYEKGVQGASSLAAWSMEPVVRRLSTQFVAANELACRGLDHLEEKIPALQYPPEKIASELKDTISTRLRSARNSISVPIASTSDKILGAALAGCELAWGMARDTAEYAANTRAGRLASGGADLALGGVEKMVEFLLPPANEESAAAPGLQDAQKPPKAKPSLVSRVGALANTLSRHTFQTTARALKQGHALAMWIPGVAPLSSLAQWGTSAAMQVVSRRRSEVRVPWLHSLTATQEEDHEEHTDTDGEETGEEEDSETEESKLSEGAALPGSQGLLGSVAHTLQKALQSTVSVVMWAPAAVLGTAGRMLHLTPAQATSSTKGRAMSLSDALKGVTDNVVDTVVNYVPLPRLSLMEPESEFRDIDNPPAEAERREAERRGSGAPPACPEPAPRPAPPRGSLRSARGLGAEERVDATALPRAAFPAVPREKPTRRVSDSFFRPSVMEPILGRAQYSQLRKKS

SEQ ID NO: 89 - protein, 292 aa, Sus scrofa
MAAAAYVDHFAAECLVSMSSRAVVHGPREGPEPGPEGAAAVAATLPHVEERRDGKDSASLFVVARILADLNQQAPAPVSAERREGAAARKARTPCRLPPAPPPAPEPASPGAGGAAAAPLSPAWSEPEPEPEAGLEPEREPGPAGSGEPSLRQRGRRGRSRADLESPQRKHKCHYAGCEKVYGKSSHLKAHLRTHTGERPFACSWQDCNKKFARSDELARHYRTHTGEKKFSCPICDKRFMRSDHLTKHARRHANFHPGMLQRRGGGSRTGSLSDYSRSDASSPTLSPASSP

SEQ ID NO: 90 - protein, 622 aa, Bison bison
MPPQLSDGLNYSAKIVRGSLDSLPQAVREFVESSAKLCRPDQVHICDGSKEENRQLLSHMEEEGVIKRLKKYDNCWLALTDPRDVARIESKTVIITREQRDTVPIPKNGLSQLGRWMSEEDFEKAFNIRFPGCMKGRTMYVIPFSMGPLGSPLSKIGIELTDSPYVVTSMRIMTRMGTSVLEALGDGEFVKCLHSVGCPLPLKKPLVNNWACNPELTLIAHLPDRREIISFGSGYGGNSLLGKKCFALRMASRLAKEEGWLAEHMLILGITNPKGQKKYFAAAFPSACGKTNLAMMNPTLPGWKVECVGDDIAWMKFDQQGNLRAINPENGFFGVAPGTSVKTNPNAIKTIQKNTIFTNVAETSDGGVYWEGIDQPLASGIKLISWKGKEWDPKDGEPCAHPNSRFCTPASQCPIIDPDWESPEGVPIEGIIFGGRRPVGVPLVYEALSWQHGVFVGAAMRSEATAAAEYKGKVIMHDPFAMRPFFGYNFGQYLAHWLSMAQRPAAKLPKIFHVNWFRKDKAGRFLWPGFGENSRVLEWMFNRVVGEGGAKLTPIGYIPDEDALDLRGLGDVDVKELFHISKEFWEEEVEEIQKYLEEQVNVDLPPEIKNQVLALKQRISQM

SEQ ID NO: 91 - protein, 591 aa, Mus musculus
MFGIQESIQRSGSSMKEEPLGSGMNAVRTWMQGAGVLDANTAAQSGVGLARAHFEKQPPSNLRKSNFFHFVLALYDRQGQPVEIERTAFVGFVEKEKEANSEKTNNGIHYRLQLLYSNGIRTEQDFYVRLIDSMTKQAIVYEGQDKNPEMCRVLLTHEIMCSRCCDKKSCGNRNETPSDPVIIDRFFLKFFLKCNQNCLKNAGNPRDMRRFQVVVSTTVNVDGHVLAVSDNMFVHNNSKHGRRARRLDPSEGTPSYLEHATPCIKAISPSEGWTTGGATVIIIGDNFFDGLQVIFGTMLVWSELITPHAIRVQTPPRHIPGVVEVTLSYKSKQFCKGTPGRFIYTALNEPTIDYGFQRLQKVIPRHPGDPERLPKEVILKRAADLVEALYGMPHNNQEIILKRAADIAEALYSVPRNHNQLPALANTSVHAGMMGVNSFSGQLAVNVSEASQATNQGFTRNSSSVSPHGYVPSTTPQQTNYNSVTTSMNGYGSAAMSNLGGSPTFLNGSAANSPYAIVPSSPTMASSTSLPSNCSSSSGIFSFSPANMVSAVKQKSAFAPVVRPQTSPPPTCTSTNGNSLQAISGMIVPPM

SEQ ID NO: 92 - protein, 451 aa, Mus musculus
MRIPVDASTSRRFTPPSTALSPGKMSEALPLGAPDGGPALASKLRSGDRSMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRRHRQKLDDQTKPGSLSFSERLSELEQLRRTAMRVSPHHPAPTPNPRASLNHSTAFNPQPQSQMQDARQIQPSPPWSYDQSYQYLGSITSSSVHPATPISPGRASGMTSLSAELSSRLSTAPDLTAFGDPRQFPTLPSISDPRMHYPGAFTYSPPVTSGIGIGMSAMSSASRYHTYLPPPYPGSSQAQAGPFQTGSPSYHLYYGASAGSYQFSMVGGERSPPRILPPCTNASTGAALLNPSLPSQSDVVETEGSHSNSPTNMPPARLEEAVWRPY

SEQ ID NO: 93 - protein, 303 aa, Mus musculus
MAATRYEPVAEIGVGAYGTVYKARDPHSGHFVALKSVRVPNGGAAGGGLPVSTVREVALLRRLEAFEHPNVVRLMDVCATSRTDRDIKVTLVFEHIDQDLRTYLDKAPPPGLPVETIKDLMRQFLSGLDFLHANCIVHRDLKPENILVTSNGTVKLADFGLARIYSYQMALTPVVVTLWYRAPEVLLQSTYATPVDMWSVGCIFAEMFRRKPLFCGNSEADQLGKIFDLIGLPPEDDWPREVSLPRGAFAPRGPRPVQSVVPEMEESGAQLLLEMLTFNPHKRISAFRALQHSYLHKEESDAE

SEQ ID NO: 94 - protein, 296 aa, Mus musculus
MHRLLAWDAACLPPPPAAFRPMEVANFYYEPDCLAYGAKAARAAPRAPAAEPAIGEHERAIDFSPYLEPLAPAADFAAPAPAHHDFLSDLFADDYGAKPSKKPADYGYVSLGRAGAKAAPPACFPPPPPAALKAEPGFEPADCKRADDAPAMAAGFPFALRAYLGYQATPSGSSGSLSTSSSSSPPGTPSPADAKAAPAACFAGPPAAPAKAKAKKTVDKLSDEYKMRRERNNIAVRKSRDKAKMRNLETQHKVLELTAENERLQKKVEQLSRELSTLRNLFKQLPEPLLASAGHC

SEQ ID NO: 95 - protein, 328 aa, Gallus gallus
MQRLVAWDAACLPIQPPAFKSMEVANFYYEADCLAALNKLHPRAAGGRSMTELTVGDHERAIDFSPYLDPLAASQQPAQPPPPAAAAGGNFEPACSSGGQDFLSDLFAEDYKGSGGGKKPDYTYISLTRHGHPCGSQSHKPGVLPGCFPPQIVETKVEPVFETLDSCKGPRKEEGGAGPGPGGMSSPYGSTVRSYLGYQSVPSGSSGNLSTSSSSSPPGTPNPSESSKSAAGAGGYSGPPAGKNKPKKCVDKHSDEYKLRRERNNIAVRKSRDKAKMRNLETQHKVLELTAENERLQKKVEQLSRELSTLRNLFKQLPEPLLASSPRC

SEQ ID NO: 96 - protein, 307 aa, Capra hircus
MQRLVAWDPACLPLPPPPPAFKSMEVANFYYEADCLAAAYGGKAAPAAPPAARPGIGEHERAIDFSPYLEPLGAPQAAPPAPAPAPASSGQHHDFLSDLFSDDYGGKNCKKAAEYGYVSLGRLGAAKGALHPGCFLKAEPGFEPADCKRKEEAGAPGGGAAGMAAGFPYALRAYLGYQAVPSGSSGSLSTSSSSSPPGTPSPADAKAPPAAAACYAGAAPVKSKAKKTVDKHSDEYKIRRERNNIAVRKSRDKAKMRNLETQHKVLELTAENERLQKKVEQLSRELSTLRNLFKQLPEPLLASSGHC

SEQ ID NO: 97 - protein, 348 aa, Bos taurus
MQRLVVWDPVCLPLPPPPPAFKSMEVANFYYEADCLAAAYGGKAAPAAPPADRPGPRPPTGELGSIGEHERAIDFSPYLEPLGAPQAPAPTTASDTFEAAPSAPAPVPASSGQHHDFLSDLFSDDYGGKNCKKAAEYGYVSLGRLGAAKGALHPGCFAPLHPPPPPPPPPAELKAEPGFEPADCKRKEEAGAPGGGAAGMAAGFPYALRAYLGYQAVPSGSSGSLSTSSSSSPPGTPSPADAKATPAAAACYAGAAPAPSQVKSKAKKTVDKHSDEYKIRRERNNIAVRKSRDKAKMRNLETQHKVLELTGENERLQKKVEQLSREVSTLRNLFKTLPEPLLASSGHC

SEQ ID NO: 98 - protein, 575 aa, Mus musculus
MFGIQDTLGRGPALKDKSLGAEMDSVRSWVRNVGVVDANVAAQSGVALSRAHFEKQPPSNLRKSNFFHFVLALYDRQGQPVEIERTAFVDFVENDKEQGNEKTNNGTHYKLQLLYSNGVRTEQDLYVRLIDSVTKQPIAYEGQNKNPEMCRVLLTHEVMCSRCCEKKSCGNRNETPSDPVIIDRFFLKFFLKCNQNCLKTAGNPRDMRRFQVVLSTTVNVDGHVLAVSDNMFVHNNSKHGRRARRLDPSEATPCIKAISPSEGWTTGGAMVIIIGDNFFDGLQVVFGTMLVWSELITPHAIRVQTPPRHIPGVVEVTLSYKSKQFCKGAPGRFIYTALNEPTIDYGFQRLQKVIPRHPGDPERLAKEMLLKRAADLVEALYGTPHNNQDIILKRAADIAEALYSVPRNPSQIPALSSSPAHSGMMGINSYGSQLGVSISESTQGNNQGYIRNTSSISPRGYSSSSTPQQSNYSTSSNSMNGYSNVPMANLGVPGSPGFLNGSPTGSPYGIMSSSPTVGSSSTSSILPFSSSVFPAVKQKSAFAPVIRPQGSPSPACSSGNGNGFRAMTGLVVPPM

SEQ ID NO: 99 - protein, 596 aa, Mus musculus
MFGIQENIPRGGTTMKEEPLGSGMNPVRSWMHTAGVVDANTAAQSGVGLARAHFEKQPPSNLRKSNFFHFVLALYDRQGQPVEIERTAFVDFVEKEKEPNNEKTNNGIHYKLQLLYSNGVRTEQDLYVRLIDSMTKQAIVYEGQDKNPEMCRVLLTHEIMCSRCCDKKSCGNRNETPSDPVIIDRFFLKFFLKCNQNCLKNAGNPRDMRRFQVVVSTTVNVDGHVLAVSDNMFVHNNSKHGRRARRLDPSEGTAPSYLENATPCIKAISPSEGWTTGGATVIIIGDNFFDGLQVVFGTMLVWSELITPHAIRVQTPPRHIPGVVEVTLSYKSKQFCKGAPGRFVYTALNEPTIDYGFQRLQKVIPRHPGDPERLPKEVLLKRAADLVEALYGMPHNNQEIILKRAADIAEALYSVPRNHNQIPTLGNTPAHTGMMGVNSFSSQLAVNVSETSQANDQVGYSRNTSSVSPRGYVPSSTPQQSNYNTVSTSMNGYGSGAMANLGVPGSPGFLNGSSANSPYGIVPSSPTMAASSVTLPSNCSSTHGIFSFSPANVISAVKQKSAFAPVVRPQASPPPSCTSANGNGLQAMSGLVVPPM